SEQUENCE LISTING (13 sequences)

SEQ ID NO: 1 - DNA, 13931 bp, Mus musculus
tacctgcctgagctccgcctccgaagaccctgtagagcaagcagcaggggctaggcccgtggccaggccacagccaggaagccaccccaccatccatccgccatgggcccacgaaagcctgccctgcggacgccgttactgctgctgttcctgctactgttcttggacaccagcgtctgggctcaagatgaagtcctggaaaacttaagcttcagctgtccaaaagatgcaactcgattcaagcacctccgaaagtacgtgtacaactatgaagctgaaagttccagcggtgtccagggcacagctgactccagaagcgccaccaagatcaactgtaaggtagagctggaggtcccccaaatctgtggtttcatcatgaggaccaaccagtgtacccttaaagaggtgtatggcttcaaccctgagggcaaggccttgatgaagaaaaccaagaactctgaagagtttgcagctgccatgtccaggtacgaactcaagctggccattcctgaagggaaacaaattgttctttaccctgacaaggatgaacctaaatatatcctgaacatcaagaggggcatcatctctgctcttctggttcccccagagacagaagaggaccaacaagagttgttcctggataccgtgtatggaaactgctcaactcaggttaccgtgaattcaagaaagggaaccgtaccaacagaaatgtccacagagagaaacctgcagcaatgtgacggcttccagcccatcagtacaagtgtcagccctctcgctctcatcaaaggcctggtccaccccttgtcaactcttatcagcagcagccaaacttgccagtacaccctggatcctaagaggaagcatgtgtctgaagctgtctgtgatgagcagcatcttttcctgcctttctcctacaagaataagtatgggatcatgacacgtgttacacagaaactgagtcttgaagacacacctaagatcaacagtcgcttcttcagtgaaggtaccaaccggatgggtctggcctttgagagcaccaagtccacgtcatccccaaagcaggctgatgctgttttgaagacccttcaagaactgaaaaaattgtccatctcagagcagaatgctcagagagcaaatctcttcaataaactggttactgagctgagaggcctcactggtgaagcaatcacatccctcttgccacagctgattgaagtgtccagccccatcactttacaagccttggttcagtgtggacagccacagtgctatactcacatcctccagtggctgaaaactgagaaggctcaccccctcctggttgacattgtcacctacctgatggctctgatcccaaatccctcaacacagaggctgcaggaaatctttaatactgccaaggagcagcagagccgagccactctgtatgcactgagccacgcagttaacagctattttgatgtggaccattcaaggagcccagttctgcaggatatcgctggttacctgttgaaacagatcgacaatgaatgcacgggcaatgaagaccacaccttcttgattctgagggtcattggaaatatgggaagaaccatggaacaagtaatgccagccctcaagtcctcagtcctgagctgtgtacgaagtacaaaaccatctctgctgattcagaaagctgctctccaggccctgaggaagatggaactggaagatgaggtccggacgatcctttttgatacatttgtaaatggtgtcgctcccgtggagaagagactggctgcctatctcttgctgatgaagaacccttcctcatcagatattaacaaaattgcccaacttctccaatgggaacagagtgagcaggtgaagaacttcgtggcatctcacattgccaacatcttgaactcggaagaactgtatgtccaagatctgaaagttttgatcaaaaatgctctggagaattctcaatttccaacgatcatggacttcagaaaattttcccgaaactatcagatttccaaatctgcttctctcccaatgttcgacccagtctcagtcaaaatagaagggaatcttatatttgatccaagcagttatcttcccagagaaagcttgctgaaaacaaccctcacagtctttggacttgcttcacttgatctctttgagattggtttagaaggaaaagggtttgagccaacactagaagctctttttggtaagcaaggattcttcccagacagtgtcaacaaggctttgtattgggtcaatggccgagttccagatggtgtctccaaggtcttggtggaccactttggctatactacagatggcaagcatgaacaggacatggtgaatggaatcatgcccattgtggacaagttgatcaaagatctgaaatctaaagaaattcctgaagccagggcctatctccgcatcctaggaaaagagctaagctttgtcagactccaagacctccaagtcctggggaagctgttgctgagtggtgcacaaactttgcagggaatcccccagatggttgtacaggccatcagagaagggtcaaagaatgacttgtttctccactacatcttcatggacaatgcctttgagctccccactggagcagggttacagctgcaagtgtcctcgtctggagtcttcacccccgggatcaaggctggtgtaagactggaattagccaacatacaggcagagctagtggcaaagccctctgtgtccttggagtttgtgacaaatatgggcatcatcatcccagacttcgctaagagcagtgtccagatgaacaccaacttcttccacgagtcaggcctggaggcgcgagtggccctgaaggctgggcagctgaaggtcatcattccttctccaaagaggccagtcaagctgttcagtggcagcaacacactgcatctggtctctaccaccaaaacagaagtgatcccacctctggttgagaacaggcagtcctggtcaacttgcaagcctctcttcactggaatgaactactgtaccacaggagcttactccaacgccagctccacggagtctgcctcttactacccactgacaggggacacaaggtatgagctggagctgaggcccacgggagaagtggagcagtattctgccactgcaacctatgaactcctaaaagaggacaagtctttggttgacacattgaagttcctagttcaagcagaaggagtgcagcagtctgaagctactgtactgttcaaatataatcggagaagcaggaccttatctagtgaagtcctaattccagggtttgatgtcaacttcgggacaatactaagagttaatgatgaatctgctaaggacaaaaacacttacaaactcatcctggacattcagaacaagaaaatcactgaggtctctctcgtgggccacttgagttatgataaaaagggagatggcaagatcaaaggtgttgtttccataccacgtttgcaagcagaagccaggagtgaggtccacacccactggtcctccaccaaactgctcttccaaatggactcatctgctacagcttacggctcaacaatttccaagagagtgacatggcgttacgataatgagataatagaatttgattggaacacgggaaccaatgtggataccaaaaaagtggcctccaatttccctgtggatctttcccattatcctagaatgttgcatgagtatgccaatggtctcctggatcacagagtccctcaaacagatgtgacttttcgggacatgggttccaaattaattgttgcaacaaacacatggcttcagatggcaaccaggggtcttccttacccccaaactctacaggatcacctcaatagcctctcagagttgaacctcctgaaaatgggactgtctgacttccatattccagacaacctcttcctaaagactgatggcagagtcaaatacacaatgaacaggaacaaaataaacattgacatccctttgcctttgggtggcaagtcttcaaaagacctcaagatgccagagagtgtgaggacaccagccctcaacttcaagtctgtgggattccatctgccatctcgagaggtccaggtccccacttttacaatccccaagacacatcagcttcaagtgcctctcttgggtgttctagacctttccacaaatgtctacagcaatttgtacaactggtcagcctcctacactggtggcaacaccagcagagaccacttcagccttcaggctcagtaccgcatgaagactgactctgtggttgacctgttttcctacagtgtgcaaggatctggagaaacaacatatgacagcaagaacacatttacattgtcctgtgatggatctctacaccataaatttctagactcaaaattcaaagtcagccacgtagaaaaatttggaaacagcccagtctcaaaaggtttactaacatttgaaacatctagtgccttgggaccacagatgtctgctactgttcacctagactcaaaaaagaaacaacatctatacgtcaaagatatcaaggttgatggacagttcagagcttcttcattttatgctcaaggcaaatatggcctgtcttgtgagagagatgttacaactggccagctgagcggcgaatccaacatgagatttaactccacctacttccagggcaccaaccagatcgtgggaatgtaccaggatggagccctgtccatcacctccacttctgacctgcaagatggcatattcaagaacacagcttccttgaaatatgaaaactatgagctgactctgaaatctgatagcagtgggcagtatgagaacttcgctgcttccaacaagctggatgtgaccttctctacgcaaagtgcactgctgcgttctgaacaccaggccaattacaagtccctgaggcttgtcacccttctttcaggatccctcacttcccagggtgtagaattaaatgctgacatcttgggcacagacaaaattaatactggtgctcacaaggcaacactaaagattgcacgtgatggactatcaaccagtgcgaccaccaacttgaagtacagccccctgctgctggagaatgagttgaatgcagagcttgggctctctggggcatccatgaaattatcaacaaacggccgcttcaaagaacaccatgcaaaattcagtcttgatgggagagctgccctcacagaggtgtcactggggagcatttaccaggccatgattctgggtgcagacagcaaaaacatcttcaacttcaaactcagccgagaagggctgaggctgtccaatgatttgatgggctcctatgctgagatgaaacttgaccacacacacagtctgaacattgcaggtctctcactggacttcttctcaaaaatggacaatatttacagtggagacaagttctataagcagaattttaacttacagctacagccctattctttcataactactttaagcaacgacctgagatatggtgctctagatttgaccaacaatggaaggtttcggctggagccactgaagctgaatgtgggtggcaactttaaaggaacctatcaaaataatgagctgaaacatatctataccatatcttatactgacctggtagtagcaagttacagagcagacactgtggctaaggttcagggtgtcgaattcagccataggctaaatgcagacattgaaggactgacttcctctgttgatgtcactaccagctacaattcagatccactgcattttaacaatgttttccacttttctctggcaccttttaccttgggcatcgacacacatacaagtggtgatgggaaactgtccttctggggagaacacactgggcagctatatagtaagtttctgttgaaagcagaacctctggcacttattgtctctcatgactacaaaggatccacaagccacagtctcccgtacgagagcagcatcagcacggctcttgaacacacagtcagtgccttgctgacgccagctgagcagacaagcacctggaaattcaagaccaaactgaatgacaaagtatacagccaggactttgaagcctacaacactaaagacaaaatcggtgttgagcttagtggacgggctgacctctctgggctgtattctccaattaaactaccgtttttctacagtgagcctgtcaatgtccttaatggcttagaggtaaatgatgctgttgacaagccccaagaattcacaattattgctgtggtgaagtacgataagaaccaggatgttcacaccatcaacctcccattcttcaaaagcctgccagactatttggagagaaatcgaagaggaatgataagtctactggaagccatgcgaggggaattgcaacgcctcagtgttgatcagtttgtgaggaaatacagagcggccctgagcagacttcctcagcagattcatcattatctgaatgcatctgactgggagagacaagtagctggtgccaaggaaaaaataacttctttcatggaaaattatagaattacagataatgatgtactaattgccatagatagtgccaaaatcaacttcaatgaaaaactctctcaacttgagacatacgcgatacaatttgatcagtatattaaagataattatgatccacatgacttaaaaagaactattgctgagattattgatcgaatcattgaaaagttaaaaattcttgatgaacagtatcatatccgtgtaaatctagcaaaatcaatccataatctctatttatttgttgaaaacgttgatcttaaccaagtcagtagtagtaacacctcttggatccaaaatgtggattccaattatcaagtcagaatccaaattcaagaaaaactacagcagctcaggacacaaattcagaatatagacattcagcagcttgctgcagaggtaaaacgacagatggacgctattgatgtcacaatgcatttagatcaattgagaactgcaattctattccaaagaataagtgacattattgaccgtgtcaaatactttgttatgaatcttattgaagattttaaagtaactgagaaaatcaatacttttagagttatagtccgtgagctaattgagaaatatgaagtagaccaacacatccaggttttaatggataaatcagtagagttggcccacagatatagcctgagcgagcctcttcagaaactcagtaatgtgctacagcgaattgagataaaagattactatgagaaattggttgggtttattgatgatactgttgagtggcttaaagcattgtctttcaaaaataccattgaagaactaaatagattgactgacatgttggtgaagaagttgaaagcatttgattatcaccagtttgtagacaaaaccaacagcaaaatccgtgagatgactcagagaatcaatgctgaaatccaagctctcaaacttccacaaaaaatggaagcattaaaactgttggtagaagacttcaaaaccacagtctccaattccctggaaagactcaaggacaccaaagtaactgtggtcattgattggctgcaggatattttgactcaaatgaaagaccatttccaagatactctggaagatgtaagagaccgaatttatcaaatggacattcagagggaactggagcacttcttgtctctggtaaaccaagtttacagtacactggtcacctatatgtctgactggtggactctgactgctaaaaacataacagactttgcagagcaatattccatccaaaactgggctgagagtataaaagtactggtggaacaaggattcatagttcctgaaatgcaaacatttctgtggaccatgcctgcttttgaggtcagtctccgtgctctccaagaaggtaactttcagacccctgtctttatagtccccttgacagatttgaggattccatcaattcggataaactttaaaatgttaaagaatataaaaatcccattgagattttccactccagaattcactcttctcaacaccttccatgtccattcctttacaattgacttgctggaaataaaagcaaagatcattagaactatcgaccaaattttgagcagtgagctacagtggcctcttccagaaatgtatttgagagacctggatgtagtgaacattcctcttgcaagactgactctgccagacttccatgtaccagaaatcacaattccagaattcacaatcccaaatgtcaatctcaaagatttacacgttcctgatcttcacataccagaattccaacttcctcacctctcacatacaattgaaatacctgcttttggcaaactgcatagcatccttaagatccaatctcctctctttatattagatgctaatgccaacatacagaatgtaacaacttcagggaacaaagcagagattgtggcttctgtcactgctaaaggagagtcccaatttgaagctctcaattttgattttcaagcacaagctcaattcctggagttaaatcctcatcctccagtcctgaaggaatccatgaacttctccagtaagcatgtgagaatggagcatgagggtgagatagtatttgatggaaaggccattgaggggaaatcagacacagtcgcaagtttacacacagagaaaaatgaagtagagtttaataatggtatgactgtcaaagtaaacaatcagctcacccttgacagtcacacaaagtacttccacaagttgagtgttcctaggctggacttctccagtaaggcttctcttaataatgaaatcaagacactattagaagctggacatgtggcattgacatcttcagggacagggtcatggaactgggcctgtcccaacttctcggatgaaggcatacattcgtcccaaattagctttactgtggatggtcccattgcttttgttggactatccaataacataaatggcaaacacttacgggtcatccaaaaactgacttatgaatctggcttcctcaactattctaagtttgaagttgagtcaaaagttgaatctcagcacgtgggctccagcattctaacagccaatggtcgggcactgctcaaggacgcaaaggcagaaatgactggtgagcacaatgccaacttaaatggaaaagttattggaactttgaaaaattctctcttcttttcagcacaaccatttgagattactgcatccacaaataatgaaggaaatttgaaagtgggttttccactaaagctgactgggaaaatagacttcctgaataactatgcattgtttctgagtccccgtgcccaacaagcaagctggcaagcgagtaccagattcaatcagtacaaatacaatcaaaacttttctgctataaacaatgaacacaacatagaagccagtataggaatgaatggagatgccaacctggatttcttaaacatacctttaacaattcctgaaattaacttgccttacacggagttcaaaactcccttactgaaggatttctccatatgggaagaaacaggcttgaaagaatttttgaagacaacaaagcaatcatttgatttgagtgtaaaggctcaatataaaaagaacagtgacaagcattccattgttgtccctctgggtatgttttatgaatttattctcaacaatgtcaattcgtgggacagaaaatttgagaaagtcagaaacaatgctttacattttcttaccacctcctataatgaagcaaaaattaaggttgataagtacaaaactgaaaattcccttaatcagccctctgggacctttcaaaatcatggctacactatcccagttgtcaacattgaagtatctccatttgctgtagagacactggcttccagccatgtgatccccacagcaataagcaccccaagtgtcacaatccctggtcctaacatcatggtgccttcatacaagttagtgctgccacccctggagttgccagttttccatggtcctgggaatctattcaagtttttcctcccagatttcaagggattcaacactattgacaatatttatattccagccatgggcaactttacctatgacttttcttttaaatcaagtgtcatcacactgaataccaatgctggactttataaccaatcagatatcgttgcccatttcctttcttcctcttcatttgtcactgacgccctgcagtacaaattagagggaacatcacgtctgatgcgaaaaaggggattgaaactagccacagctgtctctctaactaacaaatttgtaaagggcagtcatgacagcaccattagtttaaccaagaaaaacatggaagcatcagtgagaacaactgccaacctccatgctcccatattctcaatgaacttcaagcaggaacttaatggaaataccaagtcaaaacccactgtttcatcatccattgaactaaactatgacttcaattcctcaaagctgcactctactgcaacaggaggcattgatcacaagttcagcttagaaagtctcacttcctacttttccattgagtcattcaccaaaggaaatatcaagagttccttcctttctcaggaatattcaggaagtgttgccaatgaagccaatgtatatctgaattccaagggtactcggtcttcagtgaggctacaaggagcttccaaagttgatggtatctggaacgttgaagtaggagaaaattttgctggagaagccaccctccaacgcatctacaccacatgggagcacaatatgaaaaaccatttgcaggtatatagctacttcttcacaaaaggaaagcaaacatgcagagctactttggagctctccccatggaccatgtcaaccttgctacaggttcatgtgagtcaactcagttccctccttgacctccatcactttgaccaggaagtgatcctaaaagctaacactaagaaccagaagatcagctggaaaggtggggtccaggttgaatcacgggttcttcagcacaatgcacagttctccaatgaccaagaagaaatacggcttgaccttgcaggatccttagacggacagctgtgggaccttgaagctatctttttaccagtatatggcaagagcttgcaggaactcctacaaatggatggaaagcgacagtatcttcaagcttcaacttctcttctatataccaaaaaccctaatggctatctcctctcactccccgtgcaagaactggctgatagatttattataccagggataaaactaaatgacttcagtggagtaaaaatctataagaagttaagtacttcaccatttgccctcaacctaacaatgctccccaaagtaaaattccctgggattgatctgttaacacagtactctacaccagagggctcctctgtccctatttttgaggcaactatacctgaaattcatttaactgtatcccagtttacacttccaaagagccttccagttggcaacacagtctttgatctgaataagttggccaacatgattgccgatgttgacctgcctagtgtcaccctgcctgagcagactattgtaatcccacccttggagttctctgtacctgctgggatttttattcctttctttggagaactgactgcacgtgctgggatggcttctcccctgtataatgtcacttggagcgctggttggaaaaccaaagcagatcatgttgaaacgttcctagattccatgtgcacttcaaccttgcagtttctggagtatgctttaaaagttgtagaaacacacaaaattgaagaagatctgttaacctataatatcaaaggaacacttcaacactgtgacttcaatgtggagtataatgaagatggtctatttaaaggactttgggactggcagggagaggctcacctggacatcaccagcccagcactgactgactttcatctgtactacaaagaagacaagacaagtctgtctgcctcagcagcctcctcgaccatcggcactgtgggtctggattcgagcacagatgaccagagtgtggagctgaatgtctacttccacccacagtcccctccagagaagaaactcagcatattcaaaactgagtggaggtacaaggagtctgatggtgaaaggtacatcaaaattaattgggaagaagaggcagcttccagattgctaggctccctaaaaagcaatgtgcccaaggcttctaaggctatttatgattatgccaataagtaccacctggaatacgtttcttcagaactaagaaaaagtctacaggtcaatgctgaacatgccagaaggatggttgatgaaatgaacatgagtttccagagagtagcccgtgatacctaccagaatctctatgaggagatgttggctcagaagagcctgagcatccctgagaatctcaagaagagggtgttagacagtatagtacatgttactcagaagtaccacatggcagtcatgtggctgatggactcattcattcattttctgaaattcaatagagtccagttcccagggtacgctggaacatatactgtggacgaactctacactatagtcatgaaggaaaccaagaagtcactgtctcagctgtttaatgggttaggaaacctactttcctacgttcaaaaccaagtagagaaatcaagattaatcaatgacataacatttaaatgtccttttttctcaaaaccttgtaaactaaaagatctcatattgattttcagggaggagttaaacattttatcaaacataggccaacaggatatcaagtttacaacaatactaagtagtcttcagggctttttggagagagttttagacatcatagaagaacaaattaaatgcctaaaggacaatgaatctacttgtgttgctgaccatatcaacatggttttcaaaatacaggtcccatatgcttttaaatccctaagagaagacatatactttgtcctcggtgagttcaatgactttcttcaatccatacttcaggaggggtcctacaagctacagcaggtccatcagtatatgaaggcccttcgtgaagagtattttgatccgagcatggttgggtggacagtgaaatattatgaaatagaagaaaatatggttgagctgatcaagacccttttagtttcctttagggatgtctactctgaatatagtgtgacagctgctgattttgcttccaaaatgtcaactcaagttgaacaatttgtgtccagggatatcagagagtatcttagcatgcttactgatataaatggaaagtggatggaaaagattgcagagctttctattgtggcaaaggaaacaatgaaaagctgggtcactgccgtggccaaaataatgtctgattacccccagcagttccactccaatctgcaggatttttcagaccaactctctagctactatgaaaaatttgttggtgagtccacaagattgattgacctgtccattcaaaactaccacgtgtttctcagatacatcaccgagttactgagaaagctgcaggtggccacagccaataatgtgagcccctatataaagcttgctcaaggagagctgatgatcaccttctgattcatctactaacaaattcaaattaaaccttcacatagtaggagactttgtagactactataaagaccatcctgagccagacctgcagtcaacagcaagagcaagaagcacataggaactatacctgcaaccaagctggcataagaaccaagaccttcaaagcagcctgaactcaagatgacatattttacaagttagagtaaagtcaagagctgagttgttttgtccaactcaggatggagggagggagggaaggggaaataaataaatacttccttattgtgcagcaaaaaaaaaaaaaaaaaaaaaaa

SEQ ID NO: 2 - DNA, 2534 bp, Mus musculus
ggaatcccgcgccgaactcgggggcgggctgcccgggccatggcgcataaagcctctggccacctgcagggctactgctgctccggccaccgccaggcacacaccttgctgctgagggagtctcggcttctgtcatctctgtggcctccgtcacctctgtctccgtctccttcaggtcctgagccccgagagccccttccgcgcacgcggacatgggcggcagctccagggcgcgctgggtggccttggggttgggcgccctggggctgctgtttgctgcgctcggcgttgtcatgatcctcatggtgccctccctcatcaagcagcaggtgctcaagaatgtccgcatagacccgagcagcctgtccttcgggatgtggaaggagatccccgtccctttctacttgtctgtctacttcttcgaagtggtcaacccaaacgaggtcctcaacggccagaagccagtagtccgggagcgtggaccctatgtctacagggagttcagacaaaaggtcaacatcaccttcaatgacaacgacaccgtgtccttcgtggagaaccgcagcctccatttccagcctgacaagtcgcatggctcagagagtgactacattgtactgcctaacatcttggtcctggggggctcgatattgatggagagcaagcctgtgagcctgaagctgatgatgaccttggcgctggtcaccatgggccagcgtgcttttatgaaccgcacagttggtgagatcctgtggggctatgacgatcccttcgtgcattttctcaacacgtacctcccagacatgcttcccataaagggcaaatttggcctgtttgttgggatgaacaactcgaattctggggtcttcactgtcttcacgggcgtccagaatttcagcaggatccatctggtggacaaatggaacggactcagcaagatcgattattggcattcagagcagtgtaacatgatcaatgggacttccgggcagatgtgggcacccttcatgacacccgaatcctcgctggaattcttcagcccggaggcatgcaggtccatgaagctgacctacaacgaatcaagggtgtttgaaggcattcccacgtatcgcttcacggcccccgatactctgtttgccaacgggtccgtctacccacccaacgaaggcttctgcccatgccgagagtctggcattcagaatgtcagcacctgcaggtttggtgcgcctctgtttctctcccacccccacttttacaacgccgaccctgtgttgtcagaagctgttcttggtctgaaccctaacccaaaggagcattccttgttcctagacatccatccggtcactgggatccccatgaactgttctgtgaagatgcagctgagcctctacatcaaatctgtcaagggcatcgggcaaacagggaagatcgagccagtagttctgccgttgctgtggttcgaacagagcggagcaatgggtggcaagcccctgagcacgttctacacgcagctggtgctgatgccccaggttcttcactacgcgcagtatgtgctgctggggcttggaggcctcctgttgctggtgcccatcatctgccaactgcgcagccaggagaaatgctttttgttttggagtggtagtaaaaagggctcccaggataaggaggccattcaggcctactctgagtccctgatgtcaccagctgccaagggcacggtgctgcaagaagccaagctatagggtcctgaagacactataagccccccaaacctgatagcttggtcagaccagccacccagtccctacaccccgcttcttgaggactctctcagcggacagcccaccagtgccatggcctgagcccccagatgtcacacctgtccgcacgcacggcacatggatgcccacgcatgtgcaaaaacaactcagggaccagggacagacctgctgccaagtgagcctgatgggccacaggtgtgctcttctaaatggcctgtgagccaggctgtgggaactctagctgctgtcagcccctcctgtaggagctggccctgcccaggctcctgacttccctcaggaagtctttctgtctttctccatcagtctgaaagccttagttcccacagaggacggatctgtcactcctaggggctgggcatatgtcggcctcttgtgccaaggccaggcaagcagctccaggtcctgaccagtttgcacacacactctggagctgtatctggcgctttttctatcgtctctgctatgtcactgaattaaccactgtacgtggcagaggtggcaggcccctcagggtccttatttttcaggcatggggtcaaagctagaggtatgggccgtctacacccccccgccccccggcatctagtgtacctcaccagagggtattcggaggcccagcatcctgcaaccgaccccttttttctactggaagagaaattttatcatcttttgaaaggaagtcatgactgaagcaataaaccttttcactgattcaacaaaaaaaaaaaaaaa

SEQ ID NO: 3 - DNA, 6982 bp, Mus musculus
aggcattcaggcagcgagagcagagcagcgtagagcagcacagctgagctcgtgaggcaggagactcagcccgaggaaatcgcagataagtttttaattaaaaagattgagcagtaaaaagaattagaactctaaacttaagctaatagagtagcttatcgaaatattacttagtcttaataatctaagaagatcttaagagataacatgaaggcttatttaaacagtttgaaaaaggaaatgaggagaaaagtatttgtactgtataatggaggctgaccagagcagtttaggagattgtaaagggaggttttgtgaagttctaaaaggttctagtttgaaggtcggccttgtagattaaaacgaaggttacctaaatagaatctaagtggcatttaaaacagtaaagttgtagagaatagtttgaaaatgaggtgtagttttaaaagattgagaaaagtaggttaagttgacggccgttataaaaatccttcgactggcgcatgtacgtttgaaggcatgagttggaaacagggaagatggaagtgttaggctagccgggcgatggtggcgcacgcctttaatcctagcacttgggaggcagaggcaggcggatttctgagttcgaggccagcctggtctacagagtgagttccaggacagccagggctacacagagaaaccctgtcttgaaaaaacaaaaaggttaggctagtatttggagaaagaagattagaaaatggaagtgaaagacgaagaagacatacaggaaggtgaagaaaaagctgttagagaagataggaaaatagaagacaaagcatctttagaagacagaaaaggtacttaaaggcacaggtagtaggaagccgaagaatagaagatagaaagaagcaagatagaaaaacaaaatggaagttaagacaactttggatgccagcattcaagataggcaaagaagataagattgaggccaaaaggttggataagatataaagtcagaaggaaattatctttaaagccataagttcaaatttctgatggagcgagcagtttagaagagtctttagacagccacatacaagattgaagctagcaatcaaagctactaggactgaagtaaaaagttaaggcagaatgcctttgaagagttagaagaatattaaaagccttaacttgtagcttaattttgcttgatgacaaaaggacttttgataacagtttcaagattgtcagcattttgcattggacttgagctgaggtgcttttaaaatcctaacgactagcattggcagctgacccaggtctacacagaagtgcattcagtgaactaggaagacaggagcggcagacaggagtcccgaagccagtttggtgaagctaggaaggactgaggagccagcagcagcagtgcatggtgaagatagcccaggaaagagtgcggttcggtggaggaagctaggaagaaggagccatacggatgtggtggtgaagctgggaaagggttccaggatggtggagcgagagcgagttggtgatgaagctagctggcggcttggcttgtcaactgcgcggaggaggcgagcaggcattgtggagaggatagatagcggctcctagaccagcatgccagtgtgcaagaaaggctgcagggagagcatgcggtgcggtaacattccttgaggtcggcaacatggtggtggttttctgtaacttggatggtaacttgtttactttgtcttaatagttatgggggagttgtaggcttctgtgtaaagagatatatctggggctgtatgtaggcctttgcgggtgttgtaggtttttctttttcagggttatgtcctcttgcatcttgtcagaagcttttgagggctgactgccaaggcccagaaagaagaatggtagatggcaagttgtctttaaccgctcagaggggaatgaatggtagagccagcacaacctcccagttttgtaagacgttgtagtttgaacagatgacctaccacaagcctcactcctgtgtaggggaggtaattgggcaaagtgcttttgggggaatgggggcaaaatatattttgagttcttttccccttaggtctgtctagaatcctaaaggcagatgactcaagggaaccagaaaaaaggaaatccactctcaggataagcagagctcgccaggtttacagtttgtaggaagtagaggatggatgctagctttcacactgagtgtggaggagctggccatggcggaattgctggtagtttactctttccccctcccttaatgagatttgtaaaatcctaaacacttttacttgaaatatttgggagtggtcttaacagggaggagtgggtgggggaaacgttttttttctaagattttccacagatgctatagttgtgttgacacactgggttagagaaggcgtgtactgctatgctgttggcacgacaccttcagggactggagctgccttttgtccttggaagagttttcccagttgccgctgaagtcagcacagtgcggctttggttcacagtcacctcaggagaacctcaggagcttggctaggccagaggttgaagttaagttttacagcaccgtgatttaaaatatttcattaaaggggaggggtaaaacttagttggctgtggccttgtgtttgggtgggtgggggtgttaggtaattgtttagtttatgatttcagataatcataccagagaacttaaatatttggaaaaacaggaaatctcagctttcaagttggcaagtaactcccaatccagtttttgcttcttttttcctttttctttttttgaggcgggcagctaaggaaggttggttcctctgccggtccctcgaaagcgtagggcttgggggttggtctggtccactgggatgatgtgatgctacagtggggactcttctgaagctgttggatgaatatagattgtagtgtgtggttctcttttgaaatttttttcaggtgacttaatgtatcttaataactactataggaacaaaggaagtggctttaatgaccctgaaggaatttcttctggtgatagcttttatattatcaagtaagagatactatctcagttttgtataagcaagtctttttcctagtgtaggagaaatgattttccttgtgactaaacaagatgtaaaggtatgctttttttcttcttgtgcattgtatacttgtgtttatttgtaacttataatttaagaattatgataattcagcctgaatgtcttttagagggtgggcttttgttgatgagggaggggaaacctttttttttctgtagacctttttcagataacaccatctgagtcataaccagcctggcagtgtgatgacgtagatgcagagggagcagctccttggtgaatgagtgataagtaaaggcagaaaaaataatgtcatgtctccatggggaatgagcatgagccagagattgttcctactgatgaaaagctgcatatgcaaaaatttaagcaaatgaaagcaaccagtataaagttatggcaatacctttaaaagttatggcttatctaccaagctttatccacaaaagtaaagaattgatgaaaaacagtgaagatcaaatgttcatctcaaaactgcttttacaaaagcagaatagaaatgaagtgaaaatgctgcattaagcctggagtaaaaagaagctgagcttgttgagatgagtgggatcgagcggctgcgaggcggtgcagtgtgccaatgtttcgtttgcctcagacaggtttctcttcataagcagaagagttgcttcattccatctcggagcaggaaacagcagactgctgttgacagataagtgtaacttggatctgcagtattgcatgttagggatagataagtgccttttttctctttttccaaaaagacctgtagagctgttgaatgtttgcagctggcccctcttaggcagttcagaattttgagtagttttcccatccagcctcttaaaaattcctaagccttgcaccgatgggctttcatgatgggatagctaataggcttttgcatcgtaaacttcaacacaaaagcctacatgattaatgcctactttaattacattgcttacaagattaaggaatctttatcttgaagaccccatgaaagggatcattatgtgctgaaaattagatgttcatattgctaaaatttaaatgtgctccaatgtacttgtgcttaaaatcattaaattatacaaattaataaaatacttcactagagaatgtatgtatttagaaggctgtctccttatttaaataaagtcttgtttgttgtctgtagttagtgtgggcaattttggggggatgttcttctctaatcttttcagaaacttgacttcgaacacttaagtggaccagatcaggatttgagccagaagaccgaaattaactttaaggcaggaaagacaaattttattctccatgcagtgatgagcatttaataattgcaggcctggcatagaggccgtctaactaaggactaagtaccttaggcaggtgggagatgatggtcagagtaaaaggtaactacatattttgtttccagaaagtcaggggtctaatttgaccatggctaaacatctagggtaagacacttttcccccacatttccaaatatgcatgttgagtttaaatgcttacgatcatctcatccactttagccttttgtcacctcacttgagccacgagtggggtcaggcatgtgggtttaaagagttttcctttgcagagcctcatttcatccttcatggagctgctcaggactttgcatataagcgcttgcctctgtcttctgttctgctagtgagtgtgtgatgtgagaccttgcagtgagtttgtttttcctggaatgtggagggagggggggatggggcttacttgttctagctttttttttacagaccacacagaatgcaggtgtcttgacttcaggtcatgtctgttctttggcaagtaatatgtgcagtactgttccaatctgctgctattagaatgcattgtgacgcgactggagtatgattaaagaaagttgtgtttccccaagtgtttggagtagtggttgttggaggaaaagccatgagtaacaggctgagtgttgaggaaatggctctctgcagctttaagtaacccgtgtttgtgattggagccgagtccctttgctgtgctgccttaggtaaatgtttttgttcatttctggtgaggggggttgggagcactgaagcctttagtctcttccagattcaacttaaaatctgacaagaaataaatcagacaagcaacattcttgaagaaattttaactggcaagtggaaatgttttgaacagttccgtggtctttagtgcattatctttgtgtaggtgttctctctcccctcccttggtcttaattcttacatgcaggaacattgacaacagcagacatctatctattcaaggggccagagaatccagacccagtaaggaaaaatagcccatttactttaaatcgataagtgaagcagacatgccattttcagtgtggggattgggaagccctagttctttcagatgtacttcagactgtagaaggagcttccagttgaattgaaattcaccagtggacaaaatgaggacaacaggtgaacgagccttttcttgtttaagattagctactggtaatctagtgttgaatcctctccagcttcatgctggagcagctagcatgtgatgtaatgttggccttggggtggaggggtgaggtgggcgctaagcctttttttaagatttttcaggtacccctcactaaaggcactgaaggcttaatgtaggacagcggagccttcctgtgtggcaagaatcaagcaagcagtattgtatcgagaccaaagtggtatcatggtcggttttgattagcagtggggactaccctaccgtaacaccttgttggaattgaagcatccaaagaaaatacttgagaggccctgggcttgttttaacatctggaaaaaaggctgtttttatagcagcggttaccagcccaaacctcaagttgtgcttgcaggggagggaaaagggggaaagcgggcaaccagtttccccagcttttccagaatcctgttacaaggtctccccacaagtgatttctctgccacatcgccaccatgggcctttggcctaatcacagacccttcacccctcaccttgatgcagccagtagctggatccttgaggtcacgttgcatatcggtttcaaggtaaccatggtgccaaggtcctgtgggttgcaccagaaaaggccatcaattttccccttgcctgtaatttaacattaaaaccatagctaagatgttttatacatagcacctatgcagagtaaacaaaccagtatgggtatagtatgtttgataccagtgctgggtgggaatgtaggaagtcggatgaaaagcaagcctttgtaggaagttgttggggtgggattgcaaaaattctctgctaagactttttcaggtggacataacagacttggccaagctagcatcttagtggaagcagattcgtcagtagggttgtaaaggtttttcttttcctgagaaaacaaccttttgttttctcaggttttgctttttggcctttccctagctttaaaaaaaaaaaagcaaaagacgctggtggctggcactcctggtttccaggacggggttcaagtccctgcggtgtctttgcttgactcttatatcatgaggccattacatttttcttggagggttctaaaggctctgggtatggtagctgatatcactggaacactccccagcctcagtgttgaactcttgataattaactgcattgtctttcaggttatgcccaattcgtcttattacctctgagtcgacacacctcctactatttattgaatactttgattttatgaaataaaaactaaatatctctca

SEQ ID NO: 4 - RNA, 22 nt, Mus musculus
uggagugugacaaugguguuug

SEQ ID NO: 5 - DNA, 4178 bp, Mus musculus
ggaaaagcaaaaaccctttggctttgacagccaccgccacaagcctttccgcctccccagcctgcctaggtgctgggagctgggagctggattatggtggcctgagcagccgacgcagccgtaggagcccggagtccctgtcggtccccaagctgcaaagcccgcctggaagaccccgaaagctacgggctcggatagccatgcccgcccctcccagccccacaaggggcccgatccccccgctggcggccggcgtccagatgtagctgggtcccctggatcgccatcgtcgtctcctctcgtgcgctacggatttctcctgcccactctccgccgcctggaccgggaactgagcgaggggcctgcagactctgcagtcctgatgccgccgaggccgctctcctgagagaagccaccaccacccagacttaggggcaggcaagagggacagtcaccaaccggaccacaaggcccgggctcactatggccccagcgctgcactggctcctgctatgggtgggctcgggaatgctgcctgcccagggaacccatctcggcatccggctgccccttcgcagcggcctggcagggccacccctgggcctgaggctgccccgggagaccgacgaggaatcggaggagcctggccggagaggcagctttgtggagatggtggacaacctgaggggaaagtccggccagggctactatgtggagatgaccgtaggcagccccccacagacgctcaacatcctggtggacacgggcagtagtaactttgcagtgggggctgccccacaccctttcctgcatcgctactaccagaggcagctgtccagcacatatcgagacctccgaaagggtgtgtatgtgccctacacccagggcaagtgggagggggaactgggcaccgacctggtgagcatccctcatggccccaacgtcactgtgcgtgccaacattgctgccatcactgaatcggacaagttcttcatcaatggttccaactgggagggcatcctagggctggcctatgctgagattgccaggcccgacgactctttggagcccttctttgactccctggtgaagcagacccacattcccaacatcttttccctgcagctctgtggcgctggcttccccctcaaccagaccgaggcactggcctcggtgggagggagcatgatcattggtggtatcgaccactcgctatacacgggcagtctctggtacacacccatccggcgggagtggtattatgaagtgatcattgtacgtgtggaaatcaatggtcaagatctcaagatggactgcaaggagtacaactacgacaagagcattgtggacagtgggaccaccaaccttcgcttgcccaagaaagtatttgaagctgccgtcaagtccatcaaggcagcctcctcgacggagaagttcccggatggcttttggctaggggagcagctggtgtgctggcaagcaggcacgaccccttggaacattttcccagtcatttcactttacctcatgggtgaagtcaccaatcagtccttccgcatcaccatccttcctcagcaatacctacggccggtggaggacgtggccacgtcccaagacgactgttacaagttcgctgtctcacagtcatccacgggcactgttatgggagccgtcatcatggaaggtttctatgtcgtcttcgatcgagcccgaaagcgaattggctttgctgtcagcgcttgccatgtgcacgatgagttcaggacggcggcagtggaaggtccgtttgttacggcagacatggaagactgtggctacaacattccccagacagatgagtcaacacttatgaccatagcctatgtcatggcggccatctgcgccctcttcatgttgccactctgcctcatggtatgtcagtggcgctgcctgcgttgcctgcgccaccagcacgatgactttgctgatgacatctccctgctcaagtaaggaggcccgtgggcagatgatggagacgcccctggaccacatctgggtggttccctttggtcacatgagttggagctatggatggtacctgtggccagagcacctcaggaccctcaccaacctgccaatgcttctggcgtgacagaacagagaaatcaggcaagctggattacagggcttgcacctgtaggacacaggagagggaaggaagcagcgttctggtggcaggaatatccttagacaccacaaacttgagttggaaattttgctgcttgaagcttcagccctgaccctctgcccagcatcctttagagtctccaacctaaagtattctttatgtccttccagaagtactggcgtcatactcaggctacccggcatgtgtccctgtggtaccctggcagagaaagggccaatctcattccctgctggccaaagtcagcagaagaaagtgaagtttgccagttgctttagtgatagggactgcagactcaagcctacactggtacaaagactgcgtcttgagataaacaagaacctatgcgatgcgaatgtttatactcctgggggcagtcaagatgaggagacaggataggatagagacaggaaggagatggtagcaaaactgggaaaggcagaactctgatcactttctagttccaagtttagactcatctccaagacagaagcccatctggactaagaggtatcattccccaatgtgcctgtggttgtagtctgaactgaaatgaaatgggggaaaaagggcttattagccaaagagctctttttaacactcttagaggaacagtgctcatgagaaaagtcccactggacagatgaattcctatcttgttaattctgtctctctctgcttcttcaacatgctaagtggcaccaaaatgacccaaccccaaggtcttaggtgccctatgggacaacagttagaatattgtagggctagggatggtcttcccagcataggttcactccaaccaaggtgctaaaaggaacagacaggagagtcctcctctctgatccacaaaggcagagccctcaagattcatccagcagggttagggctgatgcatttgcctctgcctggattttgtttttattttctttctttttgcccagtggtacaaaacgataagctctttatggaatactgagtgggttcattcctctcttgccctctccaatggcccctctatttatctggctaaggaaacaccacgcattggctagtattaaacagcaactgtaagatagagggctttctgttctatgtcattgccttcagtatcaaggctgcctggagaaaggatggcagcctcagggcttccttactttcttctcctttcctgacagagcagcctttctgtcctgctctctgctgcccctcccaatataatccatgggtacccaggctggttcttgggctaggttgtgggggccacactcacctcttccctgccagttctaacacgacagacatgaagccagtgttagtgggaagagctgggttttcccaggatgaccactgcatcctctcctggtacgctctacactgctttcaggctggggacctgccaagtgtgggacagttgatgaggaagagacattagcagggcctctggagttgctggcccagccagctgcccacaagccataaaccaataaaataagaatcctgcgtcacagtttccagctgggtcctcttccttgccctcgcactggtgctgctctggctgagtaggaatacacccacagactgccaggaagatggagactgtccgcttccggctcagaactacagtgtaattaagcttccaggatcactaccatgaaaacgccgcattctgctttatcatttctacccatgttgggaaaaactggctttttccccatttctttacagggcaaaaaaaaaaaaaaaaaaagggagagagagagagaactcaacctagttgttatttaccctagtaactggtgttctatttttttttaaagggggaaaatttgcatttatttttcttttgatggttaactcctttgtatcataaaattatgaactctgatatgtaaaacagaaaaaaatcttgacaacagcttctcgcttgtaaaaatatgtattatacagctctattttcaaagtctcctgaaaaatgactgacctatctccactg

SEQ ID NO: 6 - DNA, 8229 bp, Mus musculus
gcggcaggatacgcgcttgggcgtcgggacgcggctgcgctcagctctctcctctcggaagctgcagccatgatggaagtttgagagttgagccgctgtgaggccaggcccggcgcaggcgagggagatgagagacggcggcggccacggcccagagcccctctcagcgcctgtgagcagccgcgggggcagcgccctcggggagccggccgggcggcggcggcggcagcggcggcgggcctcgcctcctcgtcgtctgttctaaccgggcagcttctgagcagcttcggagagagacggtggaagaagccgtgggctcgagcgggagccggcgcaggctcggcggctgcacctcccgctcctggagcgggggggagaagcggcggcggcggccgcggctccggggagggggtcggagtcgcctgtcaccattgccagggctgggaacgccggagagttgctctctccccttctcctgcctccaacacggcggcggcggcggcggcacgtccagggacccgggccggtgttaagcctcccgtccgccgccgccgcaccccccctggcccgggctccggaggccgccggaggaggcagccgctgcgaggattatccgtcttctccccattccgctgcctcggctgccaggcctctggctgctgaggagaagcaggcccagtctctgcaaccatccagcagccgccgcagcagccattacccggctgcggtccagggccaagcggcagcagagcgaggggcatcagcgaccgccaagtccagagccatttccatcctgcagaagaagcctcgccaccagcagcttctgccatctctctcctcctttttcttcagccacaggctcccagacatgacagccatcatcaaagagatcgttagcagaaacaaaaggagatatcaagaggatggattcgacttagacttgacctatatttatccaaatattattgctatgggatttcctgcagaaagacttgaaggtgtatacaggaacaatattgatgatgtagtaaggtttttggattcaaagcataaaaaccattacaagatatacaatctatgtgctgagagacattatgacaccgccaaatttaactgcagagttgcacagtatccttttgaagaccataacccaccacagctagaacttatcaaacccttctgtgaagatcttgaccaatggctaagtgaagatgacaatcatgttgcagcaattcactgtaaagctggaaagggacggactggtgtaatgatttgtgcatatttattgcatcggggcaaatttttaaaggcacaagaggccctagatttttatggggaagtaaggaccagagacaaaaagggagtcacaattcccagtcagaggcgctatgtatattattatagctacctgctaaaaaatcacctggattacagacccgtggcactgctgtttcacaagatgatgtttgaaactattccaatgttcagtggcggaacttgcaatcctcagtttgtggtctgccagctaaaggtgaagatatattcctccaattcaggacccacgcggcgggaggacaagttcatgtactttgagttccctcagccattgcctgtgtgtggtgatatcaaagtagagttcttccacaaacagaacaagatgctcaaaaaggacaaaatgtttcacttttgggtaaatacgttcttcataccaggaccagaggaaacctcagaaaaagtggaaaatggaagtctttgtgatcaggaaatcgatagcatttgcagtatagagcgtgcagataatgacaaggagtatcttgtactcaccctaacaaaaaacgatcttgacaaagcaaacaaagacaaggccaaccgatacttctctccaaattttaaggtgaaactatactttacaaaaacagtagaggagccatcaaatccagaggctagcagttcaacttctgtgactccagatgttagtgacaatgaacctgatcattatagatattctgacaccactgactctgatccagagaatgaaccttttgatgaagatcagcattcacaaattacaaaagtctgattttttttttcttatcaagagggataaaataccatgaaaaaaaaaaaacttgaataaactgaaatggaccttttttttttttttttttttttaaatggcaataggacattgtgtcagattgcagttataggaacaattctcttctcctgaccaatcttgttttaccctatacatccacagggttttgacacttgttgtccagttaaaaaaaggttgtgtagctgtgtcatgtatatacctttttgtgtcaaaaggacatttaaaattcaattaggataaataaaagatggcactttcccattttattccagttttataaaaagtggagacaggctgatgtgtatacgcaggagtttttcctttattttctgtcaccagctgaagtggctgaagagctctgattcccgggttcacgtcctacccctttgcacttgtggcaacagataagtttgcagttggctaaggaagtttctgcagggttttgttagattctaatgcatgcacttgggttgggaatggagggaatgctcagaaaggaatgtttctacctgggctctggaccatacaccatctccagctccttagatgcacctttctttagcatgctccacttactaatctggacatccgagagattggctgctgtcctgctgtttgtttgtgcattttaaagagcatattggtgctagacaaggcagctagagtgagtatatttgtagtggggtacaggaatgaaccatctacagcatcttaagaatccacaaaggaagggatataaaaaaagtggtcatagatagataaaagacacagcagcaatgacttaaccatacaaatgtggaggctttcaacaaaggatgggctggaaacagaaaatttgacaatgatttattcagtatgctttctcagttgtaatgactgctccatctcctatgtaatcaaggccagtgctaagagtcagatgctattagtccctacatcagtcaacaccttacctttatttttattaattttcaatcatatacctactgtggatgcttcatgtgctggctgccagtttgtttttctccttaaatattttataattcttcacaggaaatttcaacttgagattcaacagtaagcaggttttgttttttttttttcctagagattgatgatgcgcgtcctcagtccagtggctgtcagacgttcagcccctttgaccttacacattctattacaatgagttttgcagttttgcacattttttttaaatgtcattaactgttagggaattttacttgaatactgaatacatataatgtgtatattaaaaaagtcattgtttgtgttaaaaaagaaattagagttgcagtaaatttacagcactgcacgaataataaggcattgaagtttttcagtagaaattgtcctacagatgctttatcgacttgctattggaagaatagatcttcttaaatgtgcagtgttgagtcacttcgttatagtggtagagttgggattagggcttcaattttacttcttaaatatcattctatgtttgatatgcccagactgcatacaatttaaagcaagagtacaactactatcgtaatggtaatgtgaagatgctattacaaaggatctcctcccaacccctcgggaatttggtgtctttcaaattatatcttgaccttgacatttgaatatccagccattattagatttcttaatggtgtgaagtcccattttcaataacttattggtgctgaaattgttcactagctgtggtctgacctagttaatttacaagtacagattgcataggacccactagagaagcatttatagtttgatggtaagtagattaggcagaacgccatctaaaatattcttagaaaataatgttgatgtattttccatacctcatcagtttcactcaaccaataaagtttttaaaattgtaacaaagctcttaggatttacacatttatatttaaacattgatacatgaatattgactgactgttgataaagtcagagacaacttttcctgagatctcaccatggaaatctgtacacccccttgtctttcctaaaagctgaaagtggctgactaaaatgcaaagcagctgttgatgttttgaagatagtgataaacactgttctttgttagttttgggcacagcatgctaaactataacttgtattgttccaatatgtaacacagagggccaggtcatgaataatgacattacaatgggctgttgcactgttaatatttttcctttggaatgtgaaggtctgaatgagggttttgattttgaatgtttcagtgtttttgagaagccttgcttacattttatggtgtagtcattggaaatggaaaaatggcattatatatatattatatatatataaatatatatattatacatactctccttactttatttcagttaccatccccatagaatttgacaagaattgctatgactgaaagggttttgagtcctaattcaaactttctttatgacagtattcacgattagcctgaagtgcattctgtaggtgatctctcccgtgtttctggaatgctttcttagactcttggatgtgcagcagcttatgtgtctgaaatgacttgaaggcatcacctttaagaaggcttacagttgggccccgtacatcccaagtcctctgtaattcctcttggacatttttgccataattgtaaaagggtagttgaattaaatagcgtcaccattctttgctgtggcacaggttataaacttaagtggagtttaccggcagcatcaaatgtttcagctttaaaaataaaagtaggttacaagttacatgtttagttttagaaaatttgtgcaatatgttcataacgatggctgtggttgccacaaagtgcctcgtttacctttaaatactgttaatgtgtcgtgcatgcagacggaaggggtggatctgtgcactaaacggggggcttttactctagtattcggcagagttgccttctacctgccagctcaaaagttcgatctgttttcatatagaatatatatactaaaaccatccagtctgtaaaacagccttaccccgattcagcctcttcagatactcttgtgctgtgcagcagtggctctgtgtgtaaatgctatgcactgaggatacacaaatatgacgtgtacaggataatgcctcataccaatcagatgtccatttgttactgtgtttgttaacaaccctttatctcttagtgttataaactccacttaaaactgattaaagtctcattcttgtcattgtgtgggtgttttattaaatgagagtatttataattcaaattgcttaaatccattaaaatgttcagtaatgggcagccacatatgattacaaagttcctgtgcatttttctatttttccccctccttgctatccttccaagcaaagcatctttctgtcatcttggtagacacatacctgtctactcatggttaagaagagcactttaagccttagtcatcacttaataagttattccaggcacagtaaaaagttcaaggttcttggaaaacggtgcttatttctcttcttataagccagatgtctgaagatagccctaaccccaagaacgggcttgatgtctcaggtctgttctgtggctttctgttttttttaacactgcagttggccatcagcacatgggaggtttcatcgggacttgtccagagtagtaggctcaaatatactatctcctttctaatattcttaaaggctaaggagtcctttcaatataacagtaagataacttgtgatgttttagaagtaagcagaccattaatgtcaatgtggagtcttaatgttacatgaagttgatagtttctctgtgacccatttaaaaatacaaaccgagtagcatgcaattatgtaaagaaatatgaagattatatgtagtcacacattttctttagaattcttagtttggtgaaaacttgaatataaaggtattttgatttatatgacattttgatgatatttgaaaaaaaggaatttcctgacattttgcttttagatcatgtcccccattgtgctgtaatttaagccaacttggttcagtgaatgccatcaccatttccattgagaatttaaaactcaccagtgtttaacatgcaggcttctgagggctcccggagaatcagaccttaagcccagttgatttacttctaacgtgaaacttcgagttcctgtatactttgctagataatttgtggtacatctaaagcttagtcttaagtggcttgtgtgtggattttattcaacattcttgttgctagggtagagagaaatgttgctgagtagaaacaagagtacccagttcaatgtggtacagagagcagtccctaaaatctgtacacagtgtaatggaccactttaggagtcaagaggctgatttttcctatgaaattacattgcaacaggaagccttctagtatagttccttttactgttagaatatgtttttatgcatacgctatagctgctttcccatcttccaacaacaggtatcaggatgtaagcaagctttaaacagtgtgaagatggcaggatagtgtcatcggtaacagtcctctgactctaaatgtagttgctctgtaacactttgtgaatataacatcacaattctcatgtccttggggggggggggcatacccagtattagtatgttttagtgactaagcaatcatttttctgtttactcatgtacattttctctttaaaactaaaacctgtactgtgtatgtctccaaagccttttagcttagtttttaggaaatgaacactgaatggatcactttttagtgtagcaggtatgggatatgtgcattatagagagaccttgtcagctctctgggcctatttgaatgtttattgttggtgtgaggatggtaggggaatcagtaaatacaagttacgttggtttagcagagcaagctcagtgtgggtatttctctttgaagcgtggtgcgtgacgcactgtgagtagagaatttggtcaccctttgagtcctcttgcattttgcaaacttgctcagcaaatgcgtacctaccttgccccctaggtaaaagcaggaactactactgatttatctgtcactcagctgtctttatatgtgtgcttctgtgacttgtatcacacaagaatcttaaagatttcacaaattgttaccttttagctctgaatgttgagtattctggtgggctaacaacaagacaaactcttgacagtcatttgagaattttcatgaaacatttagctgaaaacattttataatttatgaaaaaaatgtgttaccttaaacttttacatatgtgggagacattaactgccatatttgagcatactgaattttaaatttaaaataaagctgcatatttttaaatgaaatgtttaacaaggattcatattttttgttttttaagattaaaaataatttatgtcttctcatgtggaacctcatctgtcacaatggttagattatacagaatggagcaaggcttgtagtggtttagcttacagtaaaattcttaatgtttagatgtgtttacttactggctgttatgtatacttttgagattttccacctgttctgtgtagttttctaaatgatactcctacttaaaaacagcattttagtatctattttctgtctccattaaatggtcctcattttctattgagtttggaagtgtgcacattgtgtgtgtgtgtgtgtgtgtgtgtgtgcacacgtgtgcgcgcccgtgcgtgtgtctatttgtggagtttgtatgggagaattagttttgaaagtgctagaatagagatgaaatttggttcaagtaaaattttcccactgggattttacagtttattgtaataaaatgttaattttggatgaccttgaatattaatgaatttgttagcctcttgatgtgtgcattaatgagatatatcaaagttgtatattaaaccaaagttggagttgtggaagtgtttttatgaagttccgtttggctaccaatggacataagactagaaataccttcctgtggagaatatttttcctttaaacaattaaaaaggttcattatttttgaaaaaaaaaaaaaaaaaa

SEQ ID NO: 7 - DNA, 13 bp, Artificial
  Synthetic
  misc_feature (1)..(2): LNA
  misc_feature (2)..(2): 5-methylcytosine LNA
  misc_feature (11)..(13): LNA
  misc_feature (12)..(12): 5-methylcytosine LNA
gcattggtattca

SEQ ID NO: 8 - RNA, 13 nt, Artificial
  Synthetic
  misc_feature (1)..(3): 2′-O-Me RNA
  misc_feature (1)..(1): Tocopherol-conjugated 2′-O-Me RNA
  misc_feature (12)..(13): 2′-O-Me RNA
ugaauaccaaugc

SEQ ID NO: 9 - DNA, 14121 bp, Homo sapiens
attcccaccgggacctgcggggctgagtgcccttctcggttgctgccgctgaggagcccgcccagccagccagggccgcgaggccgaggccaggccgcagcccaggagccgccccaccgcagctggcgatggacccgccgaggcccgcgctgctggcgctgctggcgctgcctgcgctgctgctgctgctgctggcgggcgccagggccgaagaggaaatgctggaaaatgtcagcctggtctgtccaaaagatgcgacccgattcaagcacctccggaagtacacatacaactatgaggctgagagttccagtggagtccctgggactgctgattcaagaagtgccaccaggatcaactgcaaggttgagctggaggttccccagctctgcagcttcatcctgaagaccagccagtgcaccctgaaagaggtgtatggcttcaaccctgagggcaaagccttgctgaagaaaaccaagaactctgaggagtttgctgcagccatgtccaggtatgagctcaagctggccattccagaagggaagcaggttttcctttacccggagaaagatgaacctacttacatcctgaacatcaagaggggcatcatttctgccctcctggttcccccagagacagaagaagccaagcaagtgttgtttctggataccgtgtatggaaactgctccactcactttaccgtcaagacgaggaagggcaatgtggcaacagaaatatccactgaaagagacctggggcagtgtgatcgcttcaagcccatccgcacaggcatcagcccacttgctctcatcaaaggcatgacccgccccttgtcaactctgatcagcagcagccagtcctgtcagtacacactggacgctaagaggaagcatgtggcagaagccatctgcaaggagcaacacctcttcctgcctttctcctacaagaataagtatgggatggtagcacaagtgacacagactttgaaacttgaagacacaccaaagatcaacagccgcttctttggtgaaggtactaagaagatgggcctcgcatttgagagcaccaaatccacatcacctccaaagcaggccgaagctgttttgaagactctccaggaactgaaaaaactaaccatctctgagcaaaatatccagagagctaatctcttcaataagctggttactgagctgagaggcctcagtgatgaagcagtcacatctctcttgccacagctgattgaggtgtccagccccatcactttacaagccttggttcagtgtggacagcctcagtgctccactcacatcctccagtggctgaaacgtgtgcatgccaacccccttctgatagatgtggtcacctacctggtggccctgatccccgagccctcagcacagcagctgcgagagatcttcaacatggcgagggatcagcgcagccgagccaccttgtatgcgctgagccacgcggtcaacaactatcataagacaaaccctacagggacccaggagctgctggacattgctaattacctgatggaacagattcaagatgactgcactggggatgaagattacacctatttgattctgcgggtcattggaaatatgggccaaaccatggagcagttaactccagaactcaagtcttcaatcctgaaatgtgtccaaagtacaaagccatcactgatgatccagaaagctgccatccaggctctgcggaaaatggagcctaaagacaaggaccaggaggttcttcttcagactttccttgatgatgcttctccgggagataagcgactggctgcctatcttatgttgatgaggagtccttcacaggcagatattaacaaaattgtccaaattctaccatgggaacagaatgagcaagtgaagaactttgtggcttcccatattgccaatatcttgaactcagaagaattggatatccaagatctgaaaaagttagtgaaagaagctctgaaagaatctcaacttccaactgtcatggacttcagaaaattctctcggaactatcaactctacaaatctgtttctcttccatcacttgacccagcctcagccaaaatagaagggaatcttatatttgatccaaataactaccttcctaaagaaagcatgctgaaaactaccctcactgcctttggatttgcttcagctgacctcatcgagattggcttggaaggaaaaggctttgagccaacattggaagctctttttgggaagcaaggatttttcccagacagtgtcaacaaagctttgtactgggttaatggtcaagttcctgatggtgtctctaaggtcttagtggaccactttggctataccaaagatgataaacatgagcaggatatggtaaatggaataatgctcagtgttgagaagctgattaaagatttgaaatccaaagaagtcccggaagccagagcctacctccgcatcttgggagaggagcttggttttgccagtctccatgacctccagctcctgggaaagctgcttctgatgggtgcccgcactctgcaggggatcccccagatgattggagaggtcatcaggaagggctcaaagaatgacttttttcttcactacatcttcatggagaatgcctttgaactccccactggagctggattacagttgcaaatatcttcatctggagtcattgctcccggagccaaggctggagtaaaactggaagtagccaacatgcaggctgaactggtggcaaaaccctccgtgtctgtggagtttgtgacaaatatgggcatcatcattccggacttcgctaggagtggggtccagatgaacaccaacttcttccacgagtcgggtctggaggctcatgttgccctaaaagctgggaagctgaagtttatcattccttccccaaagagaccagtcaagctgctcagtggaggcaacacattacatttggtctctaccaccaaaacggaggtgatcccacctctcattgagaacaggcagtcctggtcagtttgcaagcaagtctttcctggcctgaattactgcacctcaggcgcttactccaacgccagctccacagactccgcctcctactatccgctgaccggggacaccagattagagctggaactgaggcctacaggagagattgagcagtattctgtcagcgcaacctatgagctccagagagaggacagagccttggtggataccctgaagtttgtaactcaagcagaaggtgcgaagcagactgaggctaccatgacattcaaatataatcggcagagtatgaccttgtccagtgaagtccaaattccggattttgatgttgacctcggaacaatcctcagagttaatgatgaatctactgagggcaaaacgtcttacagactcaccctggacattcagaacaagaaaattactgaggtcgccctcatgggccacctaagttgtgacacaaaggaagaaagaaaaatcaagggtgttatttccataccccgtttgcaagcagaagccagaagtgagatcctcgcccactggtcgcctgccaaactgcttctccaaatggactcatctgctacagcttatggctccacagtttccaagagggtggcatggcattatgatgaagagaagattgaatttgaatggaacacaggcaccaatgtagataccaaaaaaatgacttccaatttccctgtggatctctccgattatcctaagagcttgcatatgtatgctaatagactcctggatcacagagtccctcaaacagacatgactttccggcacgtgggttccaaattaatagttgcaatgagctcatggcttcagaaggcatctgggagtcttccttatacccagactttgcaagaccacctcaatagcctgaaggagttcaacctccagaacatgggattgccagacttccacatcccagaaaacctcttcttaaaaagcgatggccgggtcaaatataccttgaacaagaacagtttgaaaattgagattcctttgccttttggtggcaaatcctccagagatctaaagatgttagagactgttaggacaccagccctccacttcaagtctgtgggattccatctgccatctcgagagttccaagtccctacttttaccattcccaagttgtatcaactgcaagtgcctctcctgggtgttctagacctctccacgaatgtctacagcaacttgtacaactggtccgcctcctacagtggtggcaacaccagcacagaccatttcagccttcgggctcgttaccacatgaaggctgactctgtggttgacctgctttcctacaatgtgcaaggatctggagaaacaacatatgaccacaagaatacgttcacactatcatgtgatgggtctctacgccacaaatttctagattcgaatatcaaattcagtcatgtagaaaaacttggaaacaacccagtctcaaaaggtttactaatattcgatgcatctagttcctggggaccacagatgtctgcttcagttcatttggactccaaaaagaaacagcatttgtttgtcaaagaagtcaagattgatgggcagttcagagtctcttcgttctatgctaaaggcacatatggcctgtcttgtcagagggatcctaacactggccggctcaatggagagtccaacctgaggtttaactcctcctacctccaaggcaccaaccagataacaggaagatatgaagatggaaccctctccctcacctccacctctgatctgcaaagtggcatcattaaaaatactgcttccctaaagtatgagaactacgagctgactttaaaatctgacaccaatgggaagtataagaactttgccacttctaacaagatggatatgaccttctctaagcaaaatgcactgctgcgttctgaatatcaggctgattacgagtcattgaggttcttcagcctgctttctggatcactaaattcccatggtcttgagttaaatgctgacatcttaggcactgacaaaattaatagtggtgctcacaaggcgacactaaggattggccaagatggaatatctaccagtgcaacgaccaacttgaagtgtagtctcctggtgctggagaatgagctgaatgcagagcttggcctctctggggcatctatgaaattaacaacaaatggccgcttcagggaacacaatgcaaaattcagtctggatgggaaagccgccctcacagagctatcactgggaagtgcttatcaggccatgattctgggtgtcgacagcaaaaacattttcaacttcaaggtcagtcaagaaggacttaagctctcaaatgacatgatgggctcatatgctgaaatgaaatttgaccacacaaacagtctgaacattgcaggcttatcactggacttctcttcaaaacttgacaacatttacagctctgacaagttttataagcaaactgttaatttacagctacagccctattctctggtaactactttaaacagtgacctgaaatacaatgctctggatctcaccaacaatgggaaactacggctagaacccctgaagctgcatgtggctggtaacctaaaaggagcctaccaaaataatgaaataaaacacatctatgccatctcttctgctgccttatcagcaagctataaagcagacactgttgctaaggttcagggtgtggagtttagccatcggctcaacacagacatcgctgggctggcttcagccattgacatgagcacaaactataattcagactcactgcatttcagcaatgtcttccgttctgtaatggccccgtttaccatgaccatcgatgcacatacaaatggcaatgggaaactcgctctctggggagaacatactgggcagctgtatagcaaattcctgttgaaagcagaacctctggcatttactttctctcatgattacaaaggctccacaagtcatcatctcgtgtctaggaaaagcatcagtgcagctcttgaacacaaagtcagtgccctgcttactccagctgagcagacaggcacctggaaactcaagacccaatttaacaacaatgaatacagccaggacttggatgcttacaacactaaagataaaattggcgtggagcttactggacgaactctggctgacctaactctactagactccccaattaaagtgccacttttactcagtgagcccatcaatatcattgatgctttagagatgagagatgccgttgagaagccccaagaatttacaattgttgcttttgtaaagtatgataaaaaccaagatgttcactccattaacctcccattttttgagaccttgcaagaatattttgagaggaatcgacaaaccattatagttgtactggaaaacgtacagagaaacctgaagcacatcaatattgatcaatttgtaagaaaatacagagcagccctgggaaaactcccacagcaagctaatgattatctgaattcattcaattgggagagacaagtttcacatgccaaggagaaactgactgctctcacaaaaaagtatagaattacagaaaatgatatacaaattgcattagatgatgccaaaatcaactttaatgaaaaactatctcaactgcagacatatatgatacaatttgatcagtatattaaagatagttatgatttacatgatttgaaaatagctattgctaatattattgatgaaatcattgaaaaattaaaaagtcttgatgagcactatcatatccgtgtaaatttagtaaaaacaatccatgatctacatttgtttattgaaaatattgattttaacaaaagtggaagtagtactgcatcctggattcaaaatgtggatactaagtaccaaatcagaatccagatacaagaaaaactgcagcagcttaagagacacatacagaatatagacatccagcacctagctggaaagttaaaacaacacattgaggctattgatgttagagtgcttttagatcaattgggaactacaatttcatttgaaagaataaatgacgttcttgagcatgtcaaacactttgttataaatcttattggggattttgaagtagctgagaaaatcaatgccttcagagccaaagtccatgagttaatcgagaggtatgaagtagaccaacaaatccaggttttaatggataaattagtagagttggcccaccaatacaagttgaaggagactattcagaagctaagcaatgtcctacaacaagttaagataaaagattactttgagaaattggttggatttattgatgatgctgtcaagaagcttaatgaattatcttttaaaacattcattgaagatgttaacaaattccttgacatgttgataaagaaattaaagtcatttgattaccaccagtttgtagatgaaaccaatgacaaaatccgtgaggtgactcagagactcaatggtgaaattcaggctctggaactaccacaaaaagctgaagcattaaaactgtttttagaggaaaccaaggccacagttgcagtgtatctggaaagcctacaggacaccaaaataaccttaatcatcaattggttacaggaggctttaagttcagcatctttggctcacatgaaggccaaattccgagagaccctagaagatacacgagaccgaatgtatcaaatggacattcagcaggaacttcaacgatacctgtctctggtaggccaggtttatagcacacttgtcacctacatttctgattggtggactcttgctgctaagaaccttactgactttgcagagcaatattctatccaagattgggctaaacgtatgaaagcattggtagagcaagggttcactgttcctgaaatcaagaccatccttgggaccatgcctgcctttgaagtcagtcttcaggctcttcagaaagctaccttccagacacctgattttatagtccccctaacagatttgaggattccatcagttcagataaacttcaaagacttaaaaaatataaaaatcccatccaggttttccacaccagaatttaccatccttaacaccttccacattccttcctttacaattgactttgtagaaatgaaagtaaagatcatcagaaccattgaccagatgctgaacagtgagctgcagtggcccgttccagatatatatctcagggatctgaaggtggaggacattcctctagcgagaatcaccctgccagacttccgtttaccagaaatcgcaattccagaattcataatcccaactctcaaccttaatgattttcaagttcctgaccttcacataccagaattccagcttccccacatctcacacacaattgaagtacctacttttggcaagctatacagtattctgaaaatccaatctcctcttttcacattagatgcaaatgctgacatagggaatggaaccacctcagcaaacgaagcaggtatcgcagcttccatcactgccaaaggagagtccaaattagaagttctcaattttgattttcaagcaaatgcacaactctcaaaccctaagattaatccgctggctctgaaggagtcagtgaagttctccagcaagtacctgagaacggagcatgggagtgaaatgctgttttttggaaatgctattgagggaaaatcaaacacagtggcaagtttacacacagaaaaaaatacactggagcttagtaatggagtgattgtcaagataaacaatcagcttaccctggatagcaacactaaatacttccacaaattgaacatccccaaactggacttctctagtcaggctgacctgcgcaacgagatcaagacactgttgaaagctggccacatagcatggacttcttctggaaaagggtcatggaaatgggcctgccccagattctcagatgagggaacacatgaatcacaaattagtttcaccatagaaggacccctcacttcctttggactgtccaataagatcaatagcaaacacctaagagtaaaccaaaacttggtttatgaatctggctccctcaacttttctaaacttgaaattcaatcacaagtcgattcccagcatgtgggccacagtgttctaactgctaaaggcatggcactgtttggagaagggaaggcagagtttactgggaggcatgatgctcatttaaatggaaaggttattggaactttgaaaaattctcttttcttttcagcccagccatttgagatcacggcatccacaaacaatgaagggaatttgaaagttcgttttccattaaggttaacagggaagatagacttcctgaataactatgcactgtttctgagtcccagtgcccagcaagcaagttggcaagtaagtgctaggttcaatcagtataagtacaaccaaaatttctctgctggaaacaacgagaacattatggaggcccatgtaggaataaatggagaagcaaatctggatttcttaaacattcctttaacaattcctgaaatgcgtctaccttacacaataatcacaactcctccactgaaagatttctctctatgggaaaaaacaggcttgaaggaattcttgaaaacgacaaagcaatcatttgatttaagtgtaaaagctcagtataagaaaaacaaacacaggcattccatcacaaatcctttggctgtgctttgtgagtttatcagtcagagcatcaaatcctttgacaggcattttgaaaaaaacagaaacaatgcattagattttgtcaccaaatcctataatgaaacaaaaattaagtttgataagtacaaagctgaaaaatctcacgacgagctccccaggacctttcaaattcctggatacactgttccagttgtcaatgttgaagtgtctccattcaccatagagatgtcggcattcggctatgtgttcccaaaagcagtcagcatgcctagtttctccatcctaggttctgacgtccgtgtgccttcatacacattaatcctgccatcattagagctgccagtccttcatgtccctagaaatctcaagctttctcttccagatttcaaggaattgtgtaccataagccatatttttattcctgccatgggcaatattacctatgatttctcctttaaatcaagtgtcatcacactgaataccaatgctgaactttttaaccagtcagatattgttgctcatctcctttcttcatcttcatctgtcattgatgcactgcagtacaaattagagggcaccacaagattgacaagaaaaaggggattgaagttagccacagctctgtctctgagcaacaaatttgtggagggtagtcataacagtactgtgagcttaaccacgaaaaatatggaagtgtcagtggcaacaaccacaaaagcccaaattccaattttgagaatgaatttcaagcaagaacttaatggaaataccaagtcaaaacctactgtctcttcctccatggaatttaagtatgatttcaattcttcaatgctgtactctaccgctaaaggagcagttgaccacaagcttagcttggaaagcctcacctcttacttttccattgagtcatctaccaaaggagatgtcaagggttcggttctttctcgggaatattcaggaactattgctagtgaggccaacacttacttgaattccaagagcacacggtcttcagtgaagctgcagggcacttccaaaattgatgatatctggaaccttgaagtaaaagaaaattttgctggagaagccacactccaacgcatatattccctctgggagcacagtacgaaaaaccacttacagctagagggcctctttttcaccaacggagaacatacaagcaaagccaccctggaactctctccatggcaaatgtcagctcttgttcaggtccatgcaagtcagcccagttccttccatgatttccctgaccttggccaggaagtggccctgaatgctaacactaagaaccagaagatcagatggaaaaatgaagtccggattcattctgggtctttccagagccaggtcgagctttccaatgaccaagaaaaggcacaccttgacattgcaggatccttagaaggacacctaaggttcctcaaaaatatcatcctaccagtctatgacaagagcttatgggatttcctaaagctggatgtaaccaccagcattggtaggagacagcatcttcgtgtttcaactgcctttgtgtacaccaaaaaccccaatggctattcattctccatccctgtaaaagttttggctgataaattcattattcctgggctgaaactaaatgatctaaattcagttcttgtcatgcctacgttccatgtcccatttacagatcttcaggttccatcgtgcaaacttgacttcagagaaatacaaatctataagaagctgagaacttcatcatttgccctcaacctaccaacactccccgaggtaaaattccctgaagttgatgtgttaacaaaatattctcaaccagaagactccttgattcccttttttgagataaccgtgcctgaatctcagttaactgtgtcccagttcacgcttccaaaaagtgtttcagatggcattgctgctttggatctaaatgcagtagccaacaagatcgcagactttgagttgcccaccatcatcgtgcctgagcagaccattgagattccctccattaagttctctgtacctgctggaattgtcattccttcctttcaagcactgactgcacgctttgaggtagactctcccgtgtataatgccacttggagtgccagtttgaaaaacaaagcagattatgttgaaacagtcctggattccacatgcagctcaaccgtacagttcctagaatatgaactaaatgttttgggaacacacaaaatcgaagatggtacgttagcctctaagactaaaggaacatttgcacaccgtgacttcagtgcagaatatgaagaagatggcaaatatgaaggacttcaggaatgggaaggaaaagcgcacctcaatatcaaaagcccagcgttcaccgatctccatctgcgctaccagaaagacaagaaaggcatctccacctcagcagcctccccagccgtaggcaccgtgggcatggatatggatgaagatgacgacttttctaaatggaacttctactacagccctcagtcctctccagataaaaaactcaccatattcaaaactgagttgagggtccgggaatctgatgaggaaactcagatcaaagttaattgggaagaagaggcagcttctggcttgctaacctctctgaaagacaacgtgcccaaggccacaggggtcctttatgattatgtcaacaagtaccactgggaacacacagggctcaccctgagagaagtgtcttcaaagctgagaagaaatctgcagaacaatgctgagtgggtttatcaaggggccattaggcaaattgatgatatcgacgtgaggttccagaaagcagccagtggcaccactgggacctaccaagagtggaaggacaaggcccagaatctgtaccaggaactgttgactcaggaaggccaagccagtttccagggactcaaggataacgtgtttgatggcttggtacgagttactcaagaattccatatgaaagtcaagcatctgattgactcactcattgattttctgaacttccccagattccagtttccggggaaacctgggatatacactagggaggaactttgcactatgttcataagggaggtagggacggtactgtcccaggtatattcgaaagtccataatggttcagaaatactgttttcctatttccaagacctagtgattacacttcctttcgagttaaggaaacataaactaatagatgtaatctcgatgtatagggaactgttgaaagatttatcaaaagaagcccaagaggtatttaaagccattcagtctctcaagaccacagaggtgctacgtaatcttcaggaccttttacaattcattttccaactaatagaagataacattaaacagctgaaagagatgaaatttacttatcttattaattatatccaagatgagatcaacacaatcttcagtgattatatcccatatgtttttaaattgttgaaagaaaacctatgccttaatcttcataagttcaatgaatttattcaaaacgagcttcaggaagcttctcaagagttacagcagatccatcaatacattatggcccttcgtgaagaatattttgatccaagtatagttggctggacagtgaaatattatgaacttgaagaaaagatagtcagtctgatcaagaacctgttagttgctcttaaggacttccattctgaatatattgtcagtgcctctaactttacttcccaactctcaagtcaagttgagcaatttctgcacagaaatattcaggaatatcttagcatccttaccgatccagatggaaaagggaaagagaagattgcagagctttctgccactgctcaggaaataattaaaagccaggccattgcgacgaagaaaataatttctgattaccaccagcagtttagatataaactgcaagatttttcagaccaactctctgattactatgaaaaatttattgctgaatccaaaagattgattgacctgtccattcaaaactaccacacatttctgatatacatcacggagttactgaaaaagctgcaatcaaccacagtcatgaacccctacatgaagcttgctccaggagaacttactatcatcctctaattttttaaaagaaatcttcatttattcttcttttccaattgaactttcacatagcacagaaaaaattcaaactgcctatattgataaaaccatacagtgagccagccttgcagtaggcagtagactataagcagaagcacatatgaactggacctgcaccaaagctggcaccagggctcggaaggtctctgaactcagaaggatggcattttttgcaagttaaagaaaatcaggatctgagttattttgctaaacttgggggaggaggaacaaataaatggagtctttattgtgtatcata

SEQ ID NO: 10 - DNA, 2759 bp, Homo sapiens
gctcaggccccgcccctgccgccggaatcctgaagcccaaggctgcccgggggcggtccggcggcgccggcgatggggcataaaaccactggccacctgccgggctgctcctgcgtgcgctgccgtcccggatccaccgtgcctctgcggcctgcgtgcccggagtccccgcctgtgtcgtctctgtcgccgtccccgtctcctgccaggcgcggagccctgcgagccgcgggtgggccccaggcgcgcagacatgggctgctccgccaaagcgcgctgggctgccggggcgctgggcgtcgcggggctactgtgcgctgtgctgggcgctgtcatgatcgtgatggtgccgtcgctcatcaagcagcaggtccttaagaacgtgcgcatcgaccccagtagcctgtccttcaacatgtggaaggagatccctatccccttctatctctccgtctacttctttgacgtcatgaaccccagcgagatcctgaagggcgagaagccgcaggtgcgggagcgcgggccctacgtgtacagggagttcaggcacaaaagcaacatcaccttcaacaacaacgacaccgtgtccttcctcgagtaccgcaccttccagttccagccctccaagtcccacggctcggagagcgactacatcgtcatgcccaacatcctggtcttgggtgcggcggtgatgatggagaataagcccatgaccctgaagctcatcatgaccttggcattcaccaccctcggcgaacgtgccttcatgaaccgcactgtgggtgagatcatgtggggctacaaggacccccttgtgaatctcatcaacaagtactttccaggcatgttccccttcaaggacaagttcggattatttgctgagctcaacaactccgactctgggctcttcacggtgttcacgggggtccagaacatcagcaggatccacctcgtggacaagtggaacgggctgagcaaggttgacttctggcattccgatcagtgcaacatgatcaatggaacttctgggcaaatgtggccgcccttcatgactcctgagtcctcgctggagttctacagcccggaggcctgccgatccatgaagctaatgtacaaggagtcaggggtgtttgaaggcatccccacctatcgcttcgtggctcccaaaaccctgtttgccaacgggtccatctacccacccaacgaaggcttctgcccgtgcctggagtctggaattcagaacgtcagcacctgcaggttcagtgcccccttgtttctctcccatcctcacttcctcaacgctgacccggttctggcagaagcggtgactggcctgcaccctaaccaggaggcacactccttgttcctggacatccacccggtcacgggaatccccatgaactgctctgtgaaactgcagctgagcctctacatgaaatctgtcgcaggcattggacaaactgggaagattgagcctgtggtcctgccgctgctctggtttgcagagagcggggccatggagggggagactcttcacacattctacactcagctggtgttgatgcccaaggtgatgcactatgcccagtacgtcctcctggcgctgggctgcgtcctgctgctggtccctgtcatctgccaaatccggagccaagagaaatgctatttattttggagtagtagtaaaaagggctcaaaggataaggaggccattcaggcctattctgaatccctgatgacatcagctcccaagggctctgtgctgcaggaagcaaaactgtagggtcctgaggacaccgtgagccagccaggcctggccgctgggcctgaccggccccccagcccctacaccccgcttctcccggactctcccagcggacagccccccagccccacagcctgagcctcccagctgccatgtgcctgttgcacacctgcacacacgccctggcacacatacacacatgcgtgcaggcttgtgcagacactcagggatggagctgctgctgaagggacttgtagggagaggctcgtcaacaagcactgttctggaaccttctctccacgtggcccacaggcctgaccacaggggctgtgggtcctgcgtccccttcctcgggtgagcctggcctgtcccgttcagccgttgggcccaggcttcctcccctccaaggtgaaacactgcagtcccggtgtggtggctccccatgcaggacgggccaggctgggagtgccgccttcctgtgccaaattcagtggggactcagtgcccaggccctggccacgagctttggccttggtctacctgccaggccaggcaaagcgcctttacacaggcctcggaaaacaatggagtgagcacaagatgccctgtgcagctgcccgagggtctccgcccaccccggccggactttgatccccccgaagtcttcacaggcactgcatcgggttgtctggcgcccttttcctccagcctaaactgacatcatcctatggactgagccggccactctctggccgaagtggccgcaggctgtgcccccgagctgcccccaccccctcacagggtccctcagattataggtgcccaggctgaggtgaagaggcctgggggccctgccttccgggcgctcctggaccctggggcaaacctgtgacccttttctactggaatagaaatgagttttatcatctttgaaaaataattcactcttgaagtaataaacgtttaaaaaaatgggaaaaaaaaaaaaaaaaaa

SEQ ID NO: 11 - DNA, 8758 bp, Homo sapiens
gtaaaggactggggccccgcaactggcctctcctgccctcttaagcgcagcgccattttagcaacgcagaagcccggcgccgggaagcctcagctcgcctgaaggcaggtcccctctgacgcctccgggagcccaggtttcccagagtccttgggacgcagcgacgagttgtgctgctatcttagctgtccttataggctggccattccaggtggtggtatttagataaaaccactcaaactctgcagtttggtcttggggtttggaggaaagcttttatttttcttcctgctccggttcagaaggtctgaagctcatacctaaccaggcataacacagaatctgcaaaacaaaaacccctaaaaaagcagacccagagcagtgtaaacacttctgggtgtgtccctgactggctgcccaaggtctctgtgtcttcggagacaaagccattcgcttagttggtctactttaaaaggccacttgaactcgctttccatggcgatttgccttgtgagcactttcaggagagcctggaagctgaaaaacggtagaaaaatttccgtgcgggccgtggggggctggcggcaactggggggccgcagatcagagtgggccactggcagccaacggcccccggggctcaggcggggagcagctctgtggtgtgggattgaggcgttttccaagagtgggttttcacgtttctaagatttcccaagcagacagcccgtgctgctccgatttctcgaacaaaaaagcaaaacgtgtggctgtcttgggagcaagtcgcaggactgcaagcagttgggggagaaagtccgccattttgccacttctcaaccgtccctgcaaggctggggctcagttgcgtaatggaaagtaaagccctgaactatcacactttaatcttccttcaaaaggtggtaaactatacctactgtccctcaagagaacacaagaagtgctttaagaggtattttaaaagttccgggggttttgtgaggtgtttgatgacccgtttaaaatatgatttccatgtttcttttgtctaaagtttgcagctcaaatctttccacacgctagtaatttaagtatttctgcatgtgtagtttgcattcaagttccataagctgttaagaaaaatctagaaaagtaaaactagaacctatttttaaccgaagaactactttttgcctccctcacaaaggcggcggaaggtgatcgaattccggtgatgcgagttgttctccgtctataaatacgcctcgcccgagctgtgcggtaggcattgaggcagccagcgcaggggcttctgctgagggggcaggcggagcttgaggaaaccgcagataagtttttttctctttgaaagatagagattaatacaactacttaaaaaatatagtcaataggttactaagatattgcttagcgttaagtttttaacgtaattttaatagcttaagattttaagagaaaatatgaagacttagaagagtagcatgaggaaggaaaagataaaaggtttctaaaacatgacggaggttgagatgaagcttcttcatggagtaaaaaatgtatttaaaagaaaattgagagaaaggactacagagccccgaattaataccaatagaagggcaatgcttttagattaaaatgaaggtgacttaaacagcttaaagtttagtttaaaagttgtaggtgattaaaataatttgaaggcgatcttttaaaaagagattaaaccgaaggtgattaaaagaccttgaaatccatgacgcagggagaattgcgtcatttaaagcctagttaacgcatttactaaacgcagacgaaaatggaaagattaattgggagtggtaggatgaaacaatttggagaagatagaagtttgaagtggaaaactggaagacagaagtacgggaaggcgaagaaaagaatagagaagatagggaaattagaagataaaaacatacttttagaagaaaaaagataaatttaaacctgaaaagtaggaagcagaagaaaaaagacaagctaggaaacaaaaagctaagggcaaaatgtacaaacttagaagaaaattggaagatagaaacaagatagaaaatgaaaatattgtcaagagtttcagatagaaaatgaaaaacaagctaagacaagtattggagaagtatagaagatagaaaaatataaagccaaaaattggataaaatagcactgaaaaaatgaggaaattattggtaaccaatttattttaaaagcccatcaatttaatttctggtggtgcagaagttagaaggtaaagcttgagaagatgagggtgtttacgtagaccagaaccaatttagaagaatacttgaagctagaaggggaagttggttaaaaatcacatcaaaaagctactaaaaggactggtgtaatttaaaaaaaactaaggcagaaggcttttggaagagttagaagaatttggaaggccttaaatatagtagcttagtttgaaaaatgtgaaggactttcgtaacggaagtaattcaagatcaagagtaattaccaacttaatgtttttgcattggactttgagttaagattattttttaaatcctgaggactagcattaattgacagctgacccaggtgctacacagaagtggattcagtgaatctaggaagacagcagcagacaggattccaggaaccagtgtttgatgaagctaggactgaggagcaagcgagcaagcagcagttcgtggtgaagataggaaaagagtccaggagccagtgcgatttggtgaaggaagctaggaagaaggaaggagcgctaacgatttggtggtgaagctaggaaaaaggattccaggaaggagcgagtgcaatttggtgatgaaggtagcaggcggcttggcttggcaaccacacggaggaggcgagcaggcgttgtgcgtagaggatcctagaccagcatgccagtgtgccaaggccacagggaaagcgagtggttggtaaaaatccgtgaggtcggcaatatgttgtttttctggaacttacttatggtaaccttttatttattttctaatataatgggggagtttcgtactgaggtgtaaagggatttatatggggacgtaggccgatttccgggtgttgtaggtttctctttttcaggcttatactcatgaatcttgtctgaagcttttgagggcagactgccaagtcctggagaaatagtagatggcaagtttgtgggttttttttttttacacgaatttgaggaaaaccaaatgaatttgatagccaaattgagacaatttcagcaaatctgtaagcagtttgtatgtttagttggggtaatgaagtatttcagttttgtgaatagatgacctgtttttacttcctcaccctgaattcgttttgtaaatgtagagtttggatgtgtaactgaggcgggggggagttttcagtatttttttttgtgggggtgggggcaaaatatgttttcagttctttttcccttaggtctgtctagaatcctaaaggcaaatgactcaaggtgtaacagaaaacaagaaaatccaatatcaggataatcagaccaccacaggtttacagtttatagaaactagagcagttctcacgttgaggtctgtggaagagatgtccattggagaaatggctggtagttactcttttttccccccacccccttaatcagactttaaaagtgcttaaccccttaaacttgttattttttacttgaagcattttgggatggtcttaacagggaagagagagggtgggggagaaaatgtttttttctaagattttccacagatgctatagtactattgacaaactgggttagagaaggagtgtaccgctgtgctgttggcacgaacaccttcagggactggagctgcttttatccttggaagagtattcccagttgaagctgaaaagtacagcacagtgcagctttggttcatattcagtcatctcaggagaacttcagaagagcttgagtaggccaaatgttgaagttaagttttccaataatgtgacttcttaaaagttttattaaaggggaggggcaaatattggcaattagttggcagtggcctgttacggttgggattggtggggtgggtttaggtaattgtttagtttatgattgcagataaactcatgccagagaacttaaagtcttagaatggaaaaagtaaagaaatatcaacttccaagttggcaagtaactcccaatgatttagtttttttccccccagtttgaattgggaagctgggggaagttaaatatgagccactgggtgtaccagtgcattaatttgggcaaggaaagtgtcataatttgatactgtatctgttttccttcaaagtatagagcttttggggaaggaaagtattgaactgggggttggtctggcctactgggctgacattaactacaattatgggaaatgcaaaagttgtttggatatggtagtgtgtggttctcttttggaatttttttcaggtgatttaataataatttaaaactactatagaaactgcagagcaaaggaagtggcttaatgatcctgaagggatttcttctgatggtagcttttgtattatcaagtaagattctattttcagttgtgtgtaagcaagtttttttttagtgtaggagaaatacttttccattgtttaactgcaaaacaagatgttaaggtatgcttcaaaaattttgtaaattgtttattttaaacttatctgtttgtaaattgtaactgattaagaattgtgatagttcagcttgaatgtctcttagagggtgggcttttgttgatgagggaggggaaactttttttttttctatagacttttttcagataacatcttctgagtcataaccagcctggcagtatgatggcctagatgcagagaaaacagctccttggtgaattgataagtaaaggcagaaaagattatatgtcatacctccattggggaataagcataaccctgagattcttactactgatgagaacattatctgcatatgccaaaaaattttaagcaaatgaaagctaccaatttaaagttacggaatctaccattttaaagttaattgcttgtcaagctataaccacaaaaataatgaattgatgagaaatacaatgaagaggcaatgtccatctcaaaatactgcttttacaaaagcagaataaaagcgaaaagaaatgaaaatgttacactacattaatcctggaataaaagaagccgaaataaatgagagatgagttgggatcaagtggattgaggaggctgtgctgtgtgccaatgtttcgtttgcctcagacaggtatctcttcgttatcagaagagttgcttcatttcatctgggagcagaaaacagcaggcagctgttaacagataagtttaacttgcatctgcagtattgcatgttagggataagtgcttatttttaagagctgtggagttcttaaatatcaaccatggcactttctcctgaccccttccctaggggatttcaggattgagaaatttttccatcgagcctttttaaaattgtaggacttgttcctgtgggcttcagtgatgggatagtacacttcactcagaggcatttgcatctttaaataatttcttaaaagcctctaaagtgatcagtgccttgatgccaactaaggaaatttgtttagcattgaatctctgaaggctctatgaaaggaatagcatgatgtgctgttagaatcagatgttactgctaaaatttacatgttgtgatgtaaattgtgtagaaaaccattaaatcattcaaaataataaactatttttattagagaatgtatacttttagaaagctgtctccttatttaaataaaatagtgtttgtctgtagttcagtgttggggcaatcttgggggggattcttctctaatctttcagaaactttgtctgcgaacactctttaatggaccagatcaggatttgagcggaagaacgaatgtaactttaaggcaggaaagacaaattttattcttcataaagtgatgagcatataataattccaggcacatggcaatagaggccctctaaataaggaataaataacctcttagacaggtgggagattatgatcagagtaaaaggtaattacacattttatttccagaaagtcaggggtctataaattgacagtgattagagtaatactttttcacatttccaaagtttgcatgttaactttaaatgcttacaatcttagagtggtaggcaatgttttacactattgaccttatatagggaagggagggggtgcctgtggggttttaaagaattttcctttgcagaggcatttcatccttcatgaagccattcaggattttgaattgcatatgagtgcttggctcttccttctgttctagtgagtgtatgagaccttgcagtgagtttatcagcatactcaaaatttttttcctggaatttggagggatgggaggagggggtggggcttacttgttgtagctttttttttttttacagacttcacagagaatgcagttgtcttgacttcaggtctgtctgttctgttggcaagtaaatgcagtactgttctgatcccgctgctattagaatgcattgtgaaacgactggagtatgattaaaagttgtgttccccaatgcttggagtagtgattgttgaaggaaaaaatccagctgagtgataaaggctgagtgttgaggaaatttctgcagttttaagcagtcgtatttgtgattgaagctgagtacattttgctggtgtatttttaggtaaaatgctttttgttcatttctggtggtgggaggggactgaagcctttagtcttttccagatgcaaccttaaaatcagtgacaagaaacattccaaacaagcaacagtcttcaagaaattaaactggcaagtggaaatgtttaaacagttcagtgatctttagtgcattgtttatgtgtgggtttctctctcccctcccttggtcttaattcttacatgcaggaacactcagcagacacacgtatgcgaagggccagagaagccagacccagtaagaaaaaatagcctatttactttaaataaaccaaacattccattttaaatgtggggattgggaaccactagttctttcagatggtattcttcagactatagaaggagcttccagttgaattcaccagtggacaaaatgaggaaaacaggtgaacaagctttttctgtatttacatacaaagtcagatcagttatgggacaatagtattgaatagatttcagctttatgctggagtaactggcatgtgagcaaactgtgttggcgtgggggtggaggggtgaggtgggcgctaagcctttttttaagatttttcaggtacccctcactaaaggcaccgaaggcttaaagtaggacaaccatggagccttcctgtggcaggagagacaacaaagcgctattatcctaaggtcaagagaagtgtcagcctcacctgatttttattagtaatgaggacttgcctcaactccctctttctggagtgaagcatccgaaggaatgcttgaagtacccctgggcttctcttaacatttaagcaagctgtttttatagcagctcttaataataaagcccaaatctcaagcggtgcttgaaggggagggaaagggggaaagcgggcaaccacttttccctagcttttccagaagcctgttaaaagcaaggtctccccacaagcaacttctctgccacatcgccaccccgtgccttttgatctagcacagacccttcacccctcacctcgatgcagccagtagcttggatccttgtgggcatgatccataatcggtttcaaggtaacgatggtgtcgaggtctttggtgggttgaactatgttagaaaaggccattaatttgcctgcaaattgttaacagaagggtattaaaaccacagctaagtagctctattataatacttatccagtgactaaaaccaacttaaaccagtaagtggagaaataacatgttcaagaactgtaatgctgggtgggaacatgtaacttgtagactggagaagataggcatttgagtggctgagagggcttttgggtgggaatgcaaaaattctctgctaagactttttcaggtgaacataacagacttggccaagctagcatcttagcggaagctgatctccaatgctcttcagtagggtcatgaaggtttttcttttcctgagaaaacaacacgtattgttttctcaggttttgctttttggcctttttctagcttaaaaaaaaaaaaagcaaaagatgctggtggttggcactcctggtttccaggacggggttcaaatccctgcggcgtctttgctttgactactaatctgtcttcaggactctttctgtatttctccttttctctgcaggtgctagttcttggagttttggggaggtgggaggtaacagcacaatatctttgaactatatacatccttgatgtataatttgtcaggagcttgacttgattgtatattcatatttacacgagaacctaatataactgccttgtctttttcaggtaatagcctgcagctggtgttttgagaagccctactgctgaaaacttaacaattttgtgtaataaaaatggagaagctctaaattgttgtggttcttttgtgaataaaaaaatcttgattggggaaaaaaaaa

SEQ ID NO: 12 - DNA, 5864 bp, Homo sapiens
acaagtctttccgcctccccagcccgcccgggagctgcgagccgcgagctggattatggtggcctgagcagccaacgcagccgcaggagcccggagcccttgcccctgcccgcgccgccgcccgccggggggaccagggaagccgccaccggcccgccatgcccgcccctcccagccccgccgggagcccgcgcccgctgcccaggctggccgccgccgtgccgatgtagcgggctccggatcccagcctctcccctgctcccgtgctctgcggatctcccctgaccgctctccacagcccggacccgggggctggcccagggccctgcaggccctggcgtcctgatgcccccaagctccctctcctgagaagccaccagcaccacccagacttgggggcaggcgccagggacggacgtgggccagtgcgagcccagagggcccgaaggccggggcccaccatggcccaagccctgccctggctcctgctgtggatgggcgcgggagtgctgcctgcccacggcacccagcacggcatccggctgcccctgcgcagcggcctggggggcgcccccctggggctgcggctgccccgggagaccgacgaagagcccgaggagcccggccggaggggcagctttgtggagatggtggacaacctgaggggcaagtcggggcagggctactacgtggagatgaccgtgggcagccccccgcagacgctcaacatcctggtggatacaggcagcagtaactttgcagtgggtgctgccccccaccccttcctgcatcgctactaccagaggcagctgtccagcacataccgggacctccggaagggtgtgtatgtgccctacacccagggcaagtgggaaggggagctgggcaccgacctggtaagcatcccccatggccccaacgtcactgtgcgtgccaacattgctgccatcactgaatcagacaagttcttcatcaacggctccaactgggaaggcatcctggggctggcctatgctgagattgccaggcctgacgactccctggagcctttctttgactctctggtaaagcagacccacgttcccaacctcttctccctgcagctttgtggtgctggcttccccctcaaccagtctgaagtgctggcctctgtcggagggagcatgatcattggaggtatcgaccactcgctgtacacaggcagtctctggtatacacccatccggcgggagtggtattatgaggtgatcattgtgcgggtggagatcaatggacaggatctgaaaatggactgcaaggagtacaactatgacaagagcattgtggacagtggcaccaccaaccttcgtttgcccaagaaagtgtttgaagctgcagtcaaatccatcaaggcagcctcctccacggagaagttccctgatggtttctggctaggagagcagctggtgtgctggcaagcaggcaccaccccttggaacattttcccagtcatctcactctacctaatgggtgaggttaccaaccagtccttccgcatcaccatccttccgcagcaatacctgcggccagtggaagatgtggccacgtcccaagacgactgttacaagtttgccatctcacagtcatccacgggcactgttatgggagctgttatcatggagggcttctacgttgtctttgatcgggcccgaaaacgaattggctttgctgtcagcgcttgccatgtgcacgatgagttcaggacggcagcggtggaaggcccttttgtcaccttggacatggaagactgtggctacaacattccacagacagatgagtcaaccctcatgaccatagcctatgtcatggctgccatctgcgccctcttcatgctgccactctgcctcatggtgtgtcagtggcgctgcctccgctgcctgcgccagcagcatgatgactttgctgatgacatctccctgctgaagtgaggaggcccatgggcagaagatagagattcccctggaccacacctccgtggttcactttggtcacaagtaggagacacagatggcacctgtggccagagcacctcaggaccctccccacccaccaaatgcctctgccttgatggagaaggaaaaggctggcaaggtgggttccagggactgtacctgtaggaaacagaaaagagaagaaagaagcactctgctggcgggaatactcttggtcacctcaaatttaagtcgggaaattctgctgcttgaaacttcagccctgaacctttgtccaccattcctttaaattctccaacccaaagtattcttcttttcttagtttcagaagtactggcatcacacgcaggttaccttggcgtgtgtccctgtggtaccctggcagagaagagaccaagcttgtttccctgctggccaaagtcagtaggagaggatgcacagtttgctatttgctttagagacagggactgtataaacaagcctaacattggtgcaaagattgcctcttgaattaaaaaaaaaaactagattgactatttatacaaatgggggcggctggaaagaggagaaggagagggagtacaaagacagggaatagtgggatcaaagctaggaaaggcagaaacacaaccactcaccagtcctagttttagacctcatctccaagatagcatcccatctcagaagatgggtgttgttttcaatgttttcttttctgtggttgcagcctgaccaaaagtgagatgggaagggcttatctagccaaagagctcttttttagctctcttaaatgaagtgcccactaagaagttccacttaacacatgaatttctgccatattaatttcattgtctctatctgaaccaccctttattctacatatgataggcagcactgaaatatcctaaccccctaagctccaggtgccctgtgggagagcaactggactatagcagggctgggctctgtcttcctggtcataggctcactctttcccccaaatcttcctctggagctttgcagccaaggtgctaaaaggaataggtaggagacctcttctatctaatccttaaaagcataatgttgaacattcattcaacagctgatgccctataacccctgcctggatttcttcctattaggctataagaagtagcaagatctttacataattcagagtggtttcattgccttcctaccctctctaatggcccctccatttatttgactaaagcatcacacagtggcactagcattataccaagagtatgagaaatacagtgctttatggctctaacattactgccttcagtatcaaggctgcctggagaaaggatggcagcctcagggcttccttatgtcctccaccacaagagctccttgatgaaggtcatctttttcccctatcctgttcttcccctccccgctcctaatggtacgtgggtacccaggctggttcttgggctaggtagtggggaccaagttcattacctccctatcagttctagcatagtaaactacggtaccagtgttagtgggaagagctgggttttcctagtatacccactgcatcctactcctacctggtcaacccgctgcttccaggtatgggacctgctaagtgtggaattacctgataagggagagggaaatacaaggagggcctctggtgttcctggcctcagccagctgcccacaagccataaaccaataaaacaagaatactgagtcagttttttatctgggttctcttcattcccactgcacttggtgctgctttggctgactgggaacaccccataactacagagtctgacaggaagactggagactgtccacttctagctcggaacttactgtgtaaataaactttcagaactgctaccatgaagtgaaaatgccacattttgctttataatttctacccatgttgggaaaaactggctttttcccagccctttccagggcataaaactcaaccccttcgatagcaagtcccatcagcctattatttttttaaagaaaacttgcacttgtttttctttttacagttacttccttcctgccccaaaattataaactctaagtgtaaaaaaaagtcttaacaacagcttcttgcttgtaaaaatatgtattatacatctgtatttttaaattctgctcctgaaaaatgactgtcccattctccactcactgcatttggggcctttcccattggtctgcatgtcttttatcattgcaggccagtggacagagggagaagggagaacaggggtcgccaacacttgtgttgctttctgactgatcctgaacaagaaagagtaacactgaggcgctcgctcccatgcacaactctccaaaacacttatcctcctgcaagagtgggctttccagggtctttactgggaagcagttaagccccctcctcaccccttccttttttctttctttactcctttggcttcaaaggattttggaaaagaaacaatatgctttacactcattttcaatttctaaatttgcaggggatactgaaaaatacggcaggtggcctaaggctgctgtaaagttgaggggagaggaaatcttaagattacaagataaaaaacgaatcccctaaacaaaaagaacaatagaactggtcttccattttgccacctttcctgttcatgacagctactaacctggagacagtaacatttcattaaccaaagaaagtgggtcacctgacctctgaagagctgagtactcaggccactccaatcaccctacaagatgccaaggaggtcccaggaagtccagctccttaaactgacgctagtcaataaacctgggcaagtgaggcaagagaaatgaggaagaatccatctgtgaggtgacaggcaaggatgaaagacaaagaaggaaaagagtatcaaaggcagaaaggagatcatttagttgggtctgaaaggaaaagtctttgctatccgacatgtactgctagtacctgtaagcattttaggtcccagaatggaaaaaaaaatcagctattggtaatataataatgtcctttccctggagtcagtttttttaaaaagttaactcttagtttttacttgtttaattctaaaagagaagggagctgaggccattccctgtaggagtaaagataaaaggataggaaaagattcaaagctctaatagagtcacagctttcccaggtataaaacctaaaattaagaagtacaataagcagaggtggaaaatgatctagttcctgatagctacccacagagcaagtgatttataaatttgaaatccaaactactttcttaatatcactttggtctccatttttcccaggacaggaaatatgtccccccctaactttcttgcttcaaaaattaaaatccagcatcccaagatcattctacaagtaattttgcacagacatctcctcaccccagtgcctgtctggagctcacccaaggtcaccaaacaacttggttgtgaaccaactgccttaaccttctgggggagggggattagctagactaggagaccagaagtgaatgggaaagggtgaggacttcacaatgttggcctgtcagagcttgattagaagccaagacagtggcagcaaaggaagacttggcccaggaaaaacctgtgggttgtgctaatttctgtccagaaaatagggtggacagaagcttgtggggtacatggaggaattgggacctggttatgttgttattctcggactgtgaattttggtgatgtaaaacagaatattctgtaaacctaatgtctgtataaataatgagcgttaacacagtaaaatattcaataagaagtcaaactactagggttaaaaaaaaaaaaaaaaaa

SEQ ID NO: 13 - DNA, 8718 bp, Homo sapiens
cctcccctcgcccggcgcggtcccgtccgcctctcgctcgcctcccgcctcccctcggtcttccgaggcgcccgggctcccggcgcggcggcggagggggcgggcaggccggcgggcggtgatgtggcgggactctttatgcgctgcggcaggatacgcgctcggcgctgggacgcgactgcgctcagttctctcctctcggaagctgcagccatgatggaagtttgagagttgagccgctgtgaggcgaggccgggctcaggcgagggagatgagagacggcggcggccgcggcccggagcccctctcagcgcctgtgagcagccgcgggggcagcgccctcggggagccggccggcctgcggcggcggcagcggcggcgtttctcgcctcctcttcgtcttttctaaccgtgcagcctcttcctcggcttctcctgaaagggaaggtggaagccgtgggctcgggcgggagccggctgaggcgcggcggcggcggcggcacctcccgctcctggagcgggggggagaagcggcggcggcggcggccgcggcggctgcagctccagggagggggtctgagtcgcctgtcaccatttccagggctgggaacgccggagagttggtctctccccttctactgcctccaacacggcggcggcggcggcggcacatccagggacccgggccggttttaaacctcccgtccgccgccgccgcaccccccgtggcccgggctccggaggccgccggcggaggcagccgttcggaggattattcgtcttctccccattccgctgccgccgctgccaggcctctggctgctgaggagaagcaggcccagtcgctgcaaccatccagcagccgccgcagcagccattacccggctgcggtccagagccaagcggcggcagagcgaggggcatcagctaccgccaagtccagagccatttccatcctgcagaagaagccccgccaccagcagcttctgccatctctctcctcctttttcttcagccacaggctcccagacatgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctgaatttttttttatcaagagggataaaacaccatgaaaataaacttgaataaactgaaaatggacctttttttttttaatggcaataggacattgtgtcagattaccagttataggaacaattctcttttcctgaccaatcttgttttaccctatacatccacagggttttgacacttgttgtccagttgaaaaaaggttgtgtagctgtgtcatgtatatacctttttgtgtcaaaaggacatttaaaattcaattaggattaataaagatggcactttcccgttttattccagttttataaaaagtggagacagactgatgtgtatacgtaggaattttttccttttgtgttctgtcaccaactgaagtggctaaagagctttgtgatatactggttcacatcctacccctttgcacttgtggcaacagataagtttgcagttggctaagagaggtttccgaagggttttgctacattctaatgcatgtattcgggttaggggaatggagggaatgctcagaaaggaaataattttatgctggactctggaccatataccatctccagctatttacacacacctttctttagcatgctacagttattaatctggacattcgaggaattggccgctgtcactgcttgttgtttgcgcatttttttttaaagcatattggtgctagaaaaggcagctaaaggaagtgaatctgtattggggtacaggaatgaaccttctgcaacatcttaagatccacaaatgaagggatataaaaataatgtcataggtaagaaacacagcaacaatgacttaaccatataaatgtggaggctatcaacaaagaatgggcttgaaacattataaaaattgacaatgatttattaaatatgttttctcaattgtaacgacttctccatctcctgtgtaatcaaggccagtgctaaaattcagatgctgttagtacctacatcagtcaacaacttacacttattttactagttttcaatcataatacctgctgtggatgcttcatgtgctgcctgcaagcttcttttttctcattaaatataaaatattttgtaatgctgcacagaaattttcaatttgagattctacagtaagcgttttttttctttgaagatttatgatgcacttattcaatagctgtcagccgttccacccttttgaccttacacattctattacaatgaattttgcagttttgcacattttttaaatgtcattaactgttagggaattttacttgaatactgaatacatataatgtttatattaaaaaggacatttgtgttaaaaaggaaattagagttgcagtaaactttcaatgctgcacacaaaaaaaagacatttgatttttcagtagaaattgtcctacatgtgctttattgatttgctattgaaagaatagggtttttttttttttttttttttttttttttaaatgtgcagtgttgaatcatttcttcatagtgctcccccgagttgggactagggcttcaatttcacttcttaaaaaaaatcatcatatatttgatatgcccagactgcatacgattttaagcggagtacaactactattgtaaagctaatgtgaagatattattaaaaaggtttttttttccagaaatttggtgtcttcaaattataccttcaccttgacatttgaatatccagccattttgtttcttaatggtataaaattccattttcaataacttattggtgctgaaattgttcactagctgtggtctgacctagttaatttacaaatacagattgaataggacctactagagcagcatttatagagtttgatggcaaatagattaggcagaacttcatctaaaatattcttagtaaataatgttgacacgttttccataccttgtcagtttcattcaacaatttttaaatttttaacaaagctcttaggatttacacatttatatttaaacattgatatatagagtattgattgattgctcataagttaaattggtaaagttagagacaactattctaacacctcaccattgaaatttatatgccaccttgtctttcataaaagctgaaaattgttacctaaaatgaaaatcaacttcatgttttgaagatagttataaatattgttctttgttacaatttcgggcaccgcatattaaaacgtaactttattgttccaatatgtaacatggagggccaggtcataaataatgacattataatgggcttttgcactgttattatttttcctttggaatgtgaaggtctgaatgagggttttgattttgaatgtttcaatgtttttgagaagccttgcttacattttatggtgtagtcattggaaatggaaaaatggcattatatatattatatatataaatatatattatacatactctccttactttatttcagttaccatccccatagaatttgacaagaattgctatgactgaaaggttttcgagtcctaattaaaactttatttatggcagtattcataattagcctgaaatgcattctgtaggtaatctctgagtttctggaatattttcttagactttttggatgtgcagcagcttacatgtctgaagttacttgaaggcatcacttttaagaaagcttacagttgggccctgtaccatcccaagtcctttgtagctcctcttgaacatgtttgccatacttttaaaagggtagttgaataaatagcatcaccattctttgctgtggcacaggttataaacttaagtggagtttaccggcagcatcaaatgtttcagctttaaaaaataaaagtagggtacaagtttaatgtttagttctagaaattttgtgcaatatgttcataacgatggctgtggttgccacaaagtgcctcgtttacctttaaatactgttaatgtgtcatgcatgcagatggaaggggtggaactgtgcactaaagtgggggctttaactgtagtatttggcagagttgccttctacctgccagttcaaaagttcaacctgttttcatatagaatatatatactaaaaaatttcagtctgttaaacagccttactctgattcagcctcttcagatactcttgtgctgtgcagcagtggctctgtgtgtaaatgctatgcactgaggatacacaaaaataccaatatgatgtgtacaggataatgcctcatcccaatcagatgtccatttgttattgtgtttgttaacaaccctttatctcttagtgttataaactccacttaaaactgattaaagtctcattcttgtcattgtgtgggtgttttattaaatgagagtttataattcaaattgcttaagtccattgaagttttaattaatgggcagccaaatgtgaatacaaagttttcagtttttttttttcctgctgtccttcaaagcctactgtttaaaaaaaaaaaaaaaaaaaaacatggcctgagagtagagtatctgtctactcatgtttaattaaggaaaaacacttatttttagggctttagtcatcacttcataaattgtataagcacattaaatagcgttctagtcctgaaaaagtccaagattcttagaaaattgtgcatatttttattatgacagatgtttgaagataattccccagaatggatttgatactttagatttcaattttgtggcttttgtctattattctgtactctgccatcagcatatggaaagcttcatttactcatcatgacttgtgccatataaaaattgatatttcggaatagtctaaaggactttttgtacttgaatttaatcatgttgtttctaatattcttaaaagcttgaagactaaagcatatcctttcaacaaagcatagtaaggtaataagaaagtgtagtttgtacaagtgttaaaaaaataaagtagacaatgttacagtgggacttattatttcaagtttacattttctccatgtaattttttaaaaagtaaatgaaaaaatgtgcaataatgtaaaatatgaagtgtatgtgtacacacattttatttttcggtatcttgggtatacgtatggttgaaaactatactggagtctaaaagtattctaatttataagaagacattttggtgatgtttgaaaaatagaaatgtgctagttttgtttttatatcatgtcctttgtacgttgtaatatgagctggcttggttcagtaaatgccatcaccatttccattgagaatttaaaactcaccagtgtttaatatgcaggcttccaaaggcttatgaaaaaaatcaagacccttaaatctagttaatttgctgctaacatgaaactctttggttcttttatttttgccagataattagacacacatctaaagcttagtcttaaatggcttaagtgtagctattgattagtgctgttgctagttcagaaagaaatgtttgtgaatggaaacaagaatattcagtccaaactgttgtaaggacagtacctgaaaaccaggaaacaggataatggaaaaagtcttttaaagatgaaatgttggagccaactttcttatagaattaattgtatgtggctatagaaagcctaatgattgttgcttatttttgagagcatattattcttttatgaccataatcttgctgtttttccatcttccaaaagatcttccttctaatatgtatatcagaatgtgggtagccagtcagacaaattcatattggttggtagctttaaaaagtttgtaatgtgaagacaggaaaggacaaaatagtttgctttggtggtagtactctggttgttaagctaggtattttgagactacttccccatcacaacaacaataaaataatcactcataatcctatcacctggagacatagccatcgttaatatgttagtgactatacaatcatgttttcttctgtatatccatgtatattctttaaaaatgaaatttatactgtacctgatctcaaagctttttagcttagtatatctgtcatgaatttgtaggatgttccattgcatcagaaaacggacagtgatttgattactttctaatgccacagatgcagattacatgtagttattgagaatcctttcgaattcagtggcttaatcatgaatgtctaaatattgttgacattaggatgatacatgtaaattaaagttacatttgtttagcatagacaagcttaacattgtagatgtttctcttcaaaaatcatcttaaacatttgcatttggaattgtgttaaatagaatgtgtgaaacactgtattagtaaacttcatcacctttctacttccttatagtttgaacttttcagtttttgtagttcccaaacagttgctcaatttagagcaaattaatttaacacctgccaaaaaaaggctgctgttggcttatcagttgtctttaaattcaaatgctcatgtgacttttatcacatcaaaaaatatttcattaatgattcacctttagctctgaaaattaccgcgtttagtaattatagtgggcttataaaaacatgcaactctttttgatagttatttgagaattttggtgaaaaatatttagctgagggcagtatagaacttataaaccaatatattgatatttttaaaacatttttacatataagtaaactgccatctttgagcataactacatttaaaaataaagctgcatatttttaaatcaagtgtttaacaagaatttatattttttattttttaaaattaaaaataatttatatttcctctgttgcatgaggattctcatctgtgcttataatggttagagattttatttgtgtggaatgaagtgaggcttgtagtcatggttctagtgtttcagtttgccaagtctgtttactgcagtgaaattcatcaaatgtttcagtgtggttttctgtagcctatcatttactggctatttttttatgtacacctttaggattttctgcctactctatccagttgtccaaatgatatcctacattttacaaatgccctttcagtttctattttctttttccattaaattgccctcatgtcctaatgtgcagtttgtaagtgtgtgtgtgtgtgtctgtgtgtgtgtgaatttgattttcaagagtgctagacttccaatttgagagattaaataatttaattcaggcaaacatttttcattggaatttcacagttcattgtaatgaaaatgttaatcctggatgacctttgacatacagtaatgaatcttggatattaatgaatttgttagtagcatcttgatgtgtgttttaatgagttattttcaaagttgtgcattaaaccaaagttggcatactggaagtgtttatatcaagttccatttggctactgatggacaaaaaatagaaatgccttcctatggagagtatttttcctttaaaaaattaaaaaggttaattattttgactaaaaaaaaaaaaaaaaaa

(19) United States
(12) Patent Application Publication
(10) Pub. No.: US 2022/0307019 A1
(43) Pub. Date: Sep. 29, 2022

(54) DOUBLE-STRANDED NUCLEIC ACID COMPLEX AND USE THEREOF

(71) Applicants: National University Corporation Tokyo Medical and Dental University, Bunkyo-ku, Tokyo (JP); WAVE LIFE SCIENCES LTD., Singapore (SG)

(72) Inventors: Takanori YOKOTA, Bunkyo-ku, Tokyo (JP); Takeshi WADA, Shinjuku-ku, Tokyo (JP); Mamoru SHIMIZU, Cambridge, MA (US)

(73) Assignees: National University Corporation Tokyo Medical and Dental University, Bunkyo-ku, Tokyo (JP); Wave Life Sciences Ltd, Singapore (SG)

(21) Appl. No.: 17/442,663

(22) PCT Filed: Mar. 25, 2020
(86) PCT No.: PCT/JP2020/013444

§ 371 Date: May 23, 2022

(30) Foreign Application Priority Data
Mar. 25, 2019 (JP) 2019-057475

(51) Int. Cl. C12N 15/113

(52) U.S. Cl. CPC C12N 15/113; C12N 2310/321; C12N 2310/31; C12N 2310/346; C12N 2310/3233; C12N 2310/3515; C12N 2310/3231; C12N 2310/341; C12N 2310/11

(57) ABSTRACT

A double-stranded nucleic acid complex is a double-stranded nucleic acid complex including a first nucleic acid strand and a second nucleic acid strand bonded to each other, the second nucleic acid strand including a complementary region having a base sequence complementary to the first nucleic acid strand; the first nucleic acid strand including natural nucleosides and non-natural nucleosides; some of the nucleosides in at least one nucleic acid strand selected from the group consisting of the first nucleic acid strand and the second nucleic acid strand being bonded together by bonds including asymmetric phosphorus atoms; and absolute configurations of the asymmetric phosphorus atoms being regulated.

### TECHNICAL FIELD

The present disclosure relates to a double-stranded nucleic acid complex, a pharmaceutical composition thereof, and various methods and reagents related thereto, including, for example, uses such as a method for treating central nervous system disorders.

### BACKGROUND ART

In recent years, there has been a high level of interest in oligonucleotides in the development of nucleic acid drugs. In particular from the viewpoints of high target gene selectivity and low toxicity, the development of nucleic acid drugs utilizing an antisense method is actively underway. So-called “antisense” nucleotides (ASOs) have nucleic acid sequences that are substantially complementary to a target sequence in a gene expression product (e.g., mRNA, miRNA), and can be used in order to alter the level or activity of a gene expression product by forming a duplex strand with the target sequence. Antisense technologies frequently involve the introduction, into cells, of an oligonucleotide (e.g., an ASO) that is complementary to a partial sequence of the mRNA (i.e., sense strand) of a target gene, selectively altering or inhibiting the expression of the protein that is encoded by the target gene. In some cases, antisense technologies involve targeting miRNAs rather than mRNAs in order to alter the activity of a target gene.

Heretofore, the present inventors have reported on the development of a double-stranded nucleic acid complex comprising an antisense oligonucleotide annealed to a complementary strand thereto (for example, see WO 2013/089283 and Kazutaka Nishina et al., DNA/RNA heteroduplex oligonucleotide for highly efficient gene silencing, NATURE COMMUNICATIONS., 2015. 1-13). In WO 2013/089283, it is disclosed that an antisense oligonucleotide annealed with a complementary strand bound to a tocopherol having a specific delivery function to a target site (liver) is delivered efficiently to the liver and has a high antisense effect.

The present inventors have also developed a double-stranded antisense nucleic acid having an exon skipping effect (for example, see WO 2014/203518) and a gapmer antisense oligonucleotide in which a wing is additionally added to the 5′-terminal, the 3′-terminal, or both the 5′-terminal and the 3′-terminal of a wing-gap-wing (gapmer) antisense oligonucleotide (for example, see WO 2014/132671).

The present inventors have further developed a double-stranded agent for delivering an oligonucleotide for treatment (for example, see WO 2014/192310).

In addition, it is known that phosphorothioate, for example, has a substantial effect on the pharmacological characteristics of ASOs (for example, see Naoki Iwamoto et al., Control of phosphorothioate stereochemistry substantially increases the efficiency of antisense oligonucleotides, nature biotechnology 2017, Vom. 35:845-851).

### SUMMARY OF INVENTION

### Technical Problem

We have considered the possibility of further developing the technologies described in above Patent Documents and Non-Patent Documents to achieve more efficient delivery of antisense oligonucleotides into a living organism, and to use the antisense oligonucleotides as therapeutic agents in the field of nucleic acid drugs. In order to do that, a designable level of suppression of the expression of a target gene and level of delivery to a target site are desired.

The present disclosure offers a double-stranded nucleic acid complex having a designable level of suppression of the expression of a target gene and the level of delivery to a target site, a composition (e.g., a pharmaceutical composition) comprising the double-stranded nucleic acid complex, and a method involving the double-stranded nucleic acid complex (e.g., a method of production or method of use).

As a result of conducting dedicated research in order to solve problems frequently encountered by ASO technologies, the present inventors discovered that the level of suppression of the expression of a target gene or the level of delivery to a target site are designable in a double-stranded nucleic acid complex that has been subjected to stereoregulation, thereby completing the present disclosure.

### Solution to Problem

The means for solving the problem described above includes the following embodiments.

<1>A double-stranded nucleic acid complex comprising a first nucleic acid strand and a second nucleic acid strand bonded to each other, the second nucleic acid strand including a complementary region having a base sequence complementary to the first nucleic acid strand,

the first nucleic acid strand including at least one selected from the group consisting of natural nucleosides and non-natural nucleosides, and

at least some of the nucleosides in at least one nucleic acid strand selected from the group consisting of the first nucleic acid strand and the second nucleic acid strand being bonded together by bonds including asymmetric phosphorus atoms, and absolute configurations of the asymmetric phosphorus atoms being regulated.

<2>The double-stranded nucleic acid complex according to <1>, wherein the double-stranded nucleic acid complex comprises a nucleic acid structure that can be recognized by RNase H.
<3>The double-stranded nucleic acid complex according to <1>or <2>, wherein the first nucleic acid strand comprises:

two terminal regions each including 2 to 10 consecutive nucleosides extending from a 5′ terminal and a 3′ terminal of the first nucleic acid strand, respectively; and

a middle region that is positioned between the terminal regions and includes at least four nucleosides,

at least some of the nucleosides in at least one region selected from the group consisting of the terminal regions and the middle region being bonded together by bonds including asymmetric phosphorus atoms, and absolute configurations of the asymmetric phosphorus atoms being regulated.

<4>The double-stranded nucleic acid complex according to <3>, wherein at least some of the nucleosides in the terminal regions are bonded by bonds including asymmetric phosphorus atoms, and an absolute configuration of each asymmetric phosphorus atom is regulated to an S-configuration or an R-configuration.
<5>The double-stranded nucleic acid complex according to <3>or <4>, wherein at least some of the nucleosides in the middle region are bonded by bonds including asymmetric phosphorus atoms, and an absolute configuration of each asymmetric phosphorus atom is regulated to an S-configuration or an R-configuration.
<6>The double-stranded nucleic acid complex according to any one of <1>to <5>, wherein the first nucleic acid strand includes at least 4 consecutive deoxyribonucleosides, the second nucleic acid strand includes at least 4 consecutive ribonucleosides, and the double-stranded nucleic acid complex comprises a structure containing at least four consecutive deoxyribonucleoside-ribonucleoside complementary base pairs.
<7>The double-stranded nucleic acid complex according to any one of <1>to <6>, wherein the first nucleic acid strand comprises:

a gap region including four or more consecutive natural nucleosides; and

a wing region including consecutive non-natural nucleosides extending from at least one region selected from the group consisting of a 5′-terminal and a 3′-terminal of the gap region.

<8>The double-stranded nucleic acid complex according to any one of <1>to <7>, wherein a bond between a non-natural nucleoside in the first nucleic acid strand and another nucleoside adjacent thereto is achieved by a bond including an asymmetric phosphorus atom, and an absolute configuration of the asymmetric phosphorus atom is regulated to an S-configuration or an R-configuration.
<9>The double-stranded nucleic acid complex according to any one of <1>to <8>, wherein the non-natural nucleosides in the first nucleic acid strand are sugar-modified nucleosides.
<10>The double-stranded nucleic acid complex according to <9>, wherein the sugar-modified nucleosides include bridged nucleosides.
<11>The double-stranded nucleic acid complex according to any one of <1>to <10>, wherein the non-natural nucleosides in the first nucleic acid strand include sugar-modified nucleosides having a 2′-O-methyl group.
<12>The double-stranded nucleic acid complex according to any one of <1>to <11>, wherein the bonds including asymmetric phosphorus atoms in at least one nucleic acid strand selected from the group consisting of the first nucleic acid strand and the second nucleic acid strand are phosphorothioate bonds.
<13>A double-stranded nucleic acid complex comprising a first nucleic acid strand and a second nucleic acid strand bonded to each other, the second nucleic acid strand including a complementary region having a base sequence complementary to the first nucleic acid strand,

the first nucleic acid strand including:

a gap region including four or more consecutive deoxyribonucleosides, and

wing regions including sugar-modified nucleosides extending from a 5′-terminal and a 3-terminal of the gap region, respectively,

at least some of the nucleosides in the first nucleic acid strand being bonded together by bonds including asymmetric phosphorus atoms, and absolute configurations of the asymmetric phosphorus atoms being regulated, and

the second nucleic acid strand including ribonucleosides.

<14>The double-stranded nucleic acid complex according to <13>, wherein bonds between nucleosides of the wing regions are bonds including asymmetric phosphorus atoms in which the absolute configurations of each asymmetric phosphorus atom is regulated to an R-configuration.
<15>The double-stranded nucleic acid complex according to <13>or <14>, wherein bonds between the deoxyribonucleosides are bonds including asymmetric phosphorus atoms in which an absolute configuration of each asymmetric phosphorus atom is regulated to an R-configuration or an S-configuration, or bonds including asymmetric phosphorus atoms in which an absolute configuration of each asymmetric phosphorus atom is not regulated.
<16>The double-stranded nucleic acid complex according to any one of <13>to <15>, wherein a base length of the gap region is from 1 to 20 bases, and a base length of each wing region is from 1 to 10 bases.
<17>The double-stranded nucleic acid complex according to any one of <13>to <16>, wherein the bonds including asymmetric phosphorus atoms are phosphorothioate bonds.
<18>The double-stranded nucleic acid complex according to any one of <1>to <17>, wherein a base length of the first nucleic acid strand is from 8 to 30 bases.
<19>The double-stranded nucleic acid complex according to any one of <1>to <18>, wherein the first nucleic acid strand further comprises at least one nucleic acid selected from the group consisting of peptide nucleic acids and morpholino nucleic acids.
<20>The double-stranded nucleic acid complex according to any one of <1>to <19>, wherein the second nucleic acid strand further comprises a functional moiety linked to at least one terminal selected from the group consisting of a 3′-terminal and a 5′-terminal of the second nucleic acid strand.
<21>The double-stranded nucleic acid complex according to <20>, wherein the functional moiety has at least one function selected from the group consisting of a labeling function, a purification function, and a targeted delivery function.
<22>The double-stranded nucleic acid complex according to <20>or <21>, wherein the functional moiety is linked to the second nucleic acid strand via a cleavable linker moiety.
<23>The double-stranded nucleic acid complex according to any one of <20>to <22>, wherein the functional moiety is at least one molecule species selected from the group consisting of a lipid, an antibody, a peptide, and a protein.
<24>The double-stranded nucleic acid complex according to <23>, wherein the lipid is at least one selected from the group consisting of cholesterol, a fatty acid, a lipid-soluble vitamin, a glycolipid, and a glyceride.
<25>The double-stranded nucleic acid complex according to <23>or <24>, wherein the lipid is at least one selected from the group consisting of cholesterol, a tocopherol, and a tocotrienol.
<26>The double-stranded nucleic acid complex according to any one of <1>to <25>, wherein the second nucleic acid strand further comprises an overhang region positioned at at least one terminal selected from the group consisting of a 5′-terminal and a 3′-terminal of the complementary region.
<27>The double-stranded nucleic acid complex according to <26>, wherein a bond between a nucleoside in the overhang region and another nucleoside adjacent thereto is achieved by a bond including an asymmetric phosphorus atom, and an absolute configuration of the asymmetric phosphorus atom is regulated to an S-configuration or an R-configuration.
<28>The double-stranded nucleic acid complex according to <26>or <27>, wherein a base length of the overhang region is at least 1 base.
<29>The double-stranded nucleic acid complex according to any one of <26>to <28>, wherein a base length of the second nucleic acid strand in the overhang region is not greater than 30 bases.
<30>The double-stranded nucleic acid complex according to any one of <26>to <29>, wherein the overhang region is not an oligonucleotide region for treatment.
<31>The double-stranded nucleic acid complex according to any one of <26>to <30>, wherein the complementary region of the second nucleic acid strand in the overhang region does not include at least two consecutive ribonucleosides.
<32>The double-stranded nucleic acid complex according to any one of <26>to <31>, wherein the overhang region includes sugar-modified nucleosides and has a base length of from 9 to 12 bases.
<33>The double-stranded nucleic acid complex according to any one of <26>to <32>, wherein the overhang region does not include sugar-modified nucleosides, and a base length of the overhang region is from 9 to 17 bases.
<34>A pharmaceutical composition comprising the double-stranded nucleic acid complex according to any one of <1>to <33>and a pharmaceutically acceptable carrier.
<35>The pharmaceutical composition according to <34>for intravenous administration, intraventricular administration, intrathecal administration, or subcutaneous administration.
<36>A method of altering a function of a transcription product in a cell, the method comprising administering the pharmaceutical composition according to <34>or <35>into the cell.
<37>A method of changing an expression level of a protein in a cell, the method comprising administering the pharmaceutical composition according to <34>or <35>into the cell.
<38>A method of changing a protein structure in a cell, the method comprising administering the pharmaceutical composition according to <34>or <35>into the cell.
<39>A use in the alteration of a function of a transcription product in a cell by administering the pharmaceutical composition according to <34>or <35>into the cell.
<40>A use in the changing of an expression level of a protein in a cell by administering the pharmaceutical composition according to <34>or <35>into the cell.
<41>A use in the changing of a protein structure in a cell by administering the pharmaceutical composition according to <34>or <35>into the cell.
<42>A method of treating a central nervous system disorder, the method comprising administering the pharmaceutical composition according to <34>or <35>into a cell.

### Advantageous Effects of Invention

With the present disclosure, it is possible to provide a double-stranded nucleic acid complex that can provide a designable level of suppression of the expression of a target gene and the level of delivery to a target site, a composition (e.g., a pharmaceutical composition) that includes the double-stranded nucleic acid complex, and a method involving the double-stranded nucleic acid complex (e.g., a method of manufacture and/or a method of use).

### BRIEF DESCRIPTION OF DRAWINGS

FIG. 1 is a drawing illustrating an example of a typical mechanism of the antisense method.

FIG. 2A is a drawing illustrating an example of one embodiment of the double-stranded nucleic acid complex according to the present disclosure.

FIG. 2B is a drawing illustrating an example of one embodiment of the double-stranded nucleic acid complex according to the present disclosure.

FIG. 2C is a drawing illustrating an example of one embodiment of the double-stranded nucleic acid complex according to the present disclosure.

FIG. 3 is a drawing illustrating the structures of various natural nucleosides or non-natural nucleosides.

FIG. 4 is a graph illustrating the results of experiments described in Embodiments 1 to 6 and Comparative Example 1, wherein the target gene (ApoB) expression suppressing effects of the nucleic acid complex according to the present invention were compared.

FIG. 5 is a graph illustrating the results of experiments described in Embodiments 1 to 6 and Comparative Example 1, wherein the levels of transfer of the nucleic acid complex according to the present invention to a target site were compared.

### DESCRIPTION OF EMBODIMENTS

In this specification, the numerical ranges indicated using “-” indicate ranges including the numerical values listed on either side of the “-” as the minimum and maximum values, respectively. In the numerical ranges described stepwise in this specification, the upper limit or lower limit described for a give numerical range may be substituted for an upper limit or lower limit of the numerical range of another stepwise description. In addition, in the numerical ranges described in this specification, the upper limit or lower limit described for a give numerical range may be substituted for the values indicated in the embodiments.

In this specification, when there are a plurality of substances corresponding to each component in a composition, the amount of each component in the composition indicates the total amount of the plurality of substances present in the composition unless specified otherwise.

In this specification, combinations of preferable modes are more preferable modes.

In this specification, the term “nucleic acid” is used synonymously with a polynucleotide and an oligonucleotide and refers to a nucleotide polymer or oligomer of any length.

The term “nucleic acid strand,” “nucleotide strand,” or “strand” in this specification is also used to indicate an oligonucleotide in this specification.

The term “nucleic acid base” or “base” indicates a heterocyclic moiety that can be paired with a base of another nucleic acid. The term “complementary” in this specification refers to a relationship in which so-called Watson-Crick base pairs (i.e., natural type base pairs) or non-Watson-Crick base pairs (e.g., Hoogsteen base pairs and the like) can be formed via hydrogen bonding.

In this specification, a heteroduplex oligonucleotide may be referred to as an “HDO,” and an antisense oligonucleotide may be referred to as an “ASO.”

### Double-Stranded Nucleic Acid Complex

The double-stranded nucleic acid complex according to the present disclosure is a double-stranded nucleic acid complex comprising a first nucleic acid strand and a second nucleic acid strand bonded to each other, the second nucleic acid strand including a complementary region having a base sequence complementary to the first nucleic acid strand; the first nucleic acid strand including at least one selected from the group consisting of natural nucleosides and non-natural nucleosides; and at least some of the nucleosides in at least one nucleic acid strand selected from the group consisting of the first nucleic acid strand and the second nucleic acid strand being bonded together by bonds including asymmetric phosphorus atoms, and absolute configurations of the asymmetric phosphorus atoms being regulated (also simply called “stereoregulation of asymmetric phosphorus atoms” hereafter). The first nucleic acid strand of the double-stranded nucleic acid complex according to the present disclosure may preferably include both natural nucleosides and non-natural nucleosides.

The oligonucleotides contained in the double-stranded nucleic acid complex of this disclosure, as descried in the specification, contains stereoregulated asymmetric phosphorus atoms and also may have other defining features.

Since the double-stranded nucleic acid complex according to the present disclosure is configured so that at least some of the nucleosides in at least one nucleic acid strand selected from the group consisting of the first nucleic acid strand and the second nucleic acid strand are bonded by bonds including asymmetric phosphorus atoms, and the asymmetric phosphorus atoms are stereoregulated, the level of suppression of the expression of a target gene and the level of delivery to a target site are designable.

### Bonds Including Asymmetric Phosphorus Atoms

In the double-stranded nucleic acid complex according to the present disclosure, the asymmetric phosphorus atoms in the bonds including asymmetric phosphorus atoms in at least one nucleic acid strand selected from the group consisting of the first nucleic acid strand and the second nucleic acid strand are stereoregulated. More specifically, at least one nucleic acid strand selected from the group consisting of the first nucleic acid strand and the second nucleic acid strand is bonded by bonds including asymmetric phosphorus atoms each regulated to one of two configurations (R-configuration or S-configuration) with the phosphorus atom serving as a center of asymmetry.

In the present disclosure, regulating an absolute configuration of phosphorus to the R-configuration may be called “regulating to Rp,” and regulating an absolute configuration to the S-configuration may be called “regulating to Sp.”

For example, a double-stranded nucleic acid complex including RNA and DNA serves as a substrate for RNase H inside cells and therefore yields a greater antisense effect inside cells, which makes it possible to suppress the expression of a target gene, but since the absolute configurations of asymmetric phosphorus atoms are stereoregulated, activities such as the adjustment of nuclease resistance, RNase H activity, protein binding, and lipophilicity can be controlled, and these activities can be further enhanced.

In the present disclosure, the “antisense effect” means to suppress or reduce the expression of a target gene or the level of a transcription product of a targeted gene such as a protein, which occurs as a result of formation of a duplex strand of a transcription product such as an RNA sense strand and an antisense oligonucleotide described herein (for example, formation of a duplex strand can alter RNA editing such as splicing, RNA-protein binding, RNA degradation resulting from RNase H degradation, or RNA translation, such as translation to a protein.)

In the present disclosure, the “antisense effect” means to suppress or reduce the expression of a target gene or a target gene transcription product (e.g., an RNA sense strand or protein) arising as the result of hybridization of an antisense oligonucleotide, for example, RNA translation to protein, RNA-protein binding, RNA digestion by RNase H, or other gene expression products (RNA sense strand).

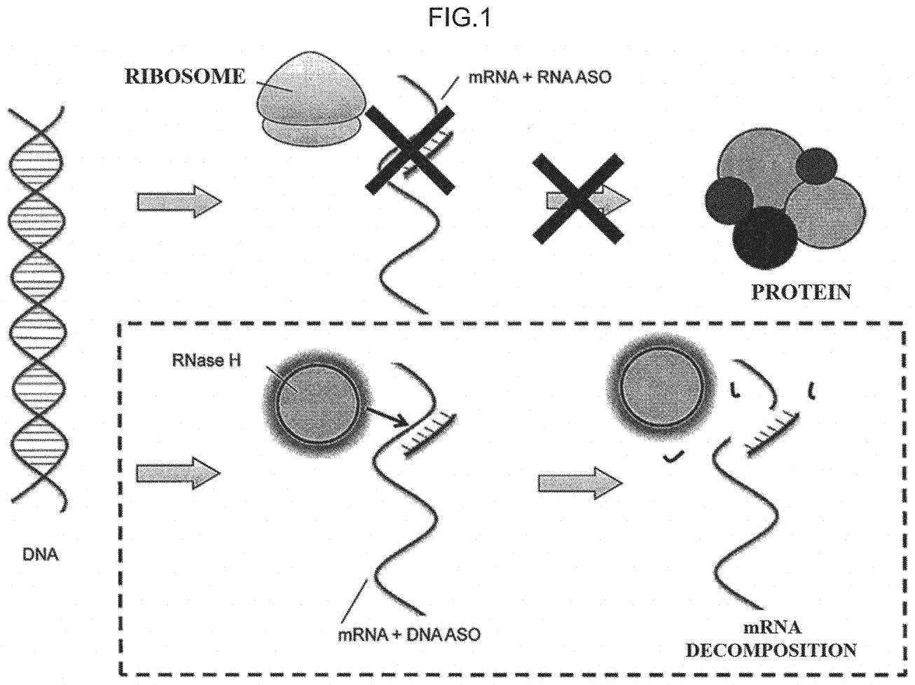

Inhibition of translation or a splicing function modifying effect such as exon skipping, for example, may be caused by hybridization into the transcription product of (for example, the first nucleic acid strand) of the antisense oligonucleotide (see the description in the upper part outside the area surrounded by dotted lines in FIG. 1). Alternatively, decomposition of the transcription product may occur as a result of recognition of the hybridized portion (see the description within the area surrounded by dotted lines in FIG. 1).

For example, in the inhibition of translation, when an oligonucleotide containing RNA is introduced into a cell as an antisense oligonucleotide (ASO), the ASO bonds to the transcription product (mRNA) of the target gene, and a partial double strand is formed. This double strand fulfills the roll of as cover for obstructing translation by a ribosome, so the expression of a protein encoded by the target gene is inhibited (upper part of FIG. 1). On the other hand, when an oligonucleotide containing DNA is introduced into a cell as an ASO, a partial DNA-RNA heteroduplex is formed. This structure is recognized by RNase H, and as a result, the mRNA of the target gene is decomposed. Therefore, the expression of a protein encoded by the target gene is inhibited (lower part of FIG. 1), which is called an RNase H-dependent route. Further, the antisense effect may be imparted, for example, by targeting the intron of pre-mRNA. The antisense effect may also be imparted by targeting miRNA, and in this case, the function of the miRNA is inhibited so that the expression of the gene ordinarily regulated by the miRNA may be enhanced.

An “antisense oligonucleotide” or an “antisense nucleic acid” refers to a single-stranded oligonucleotide which contains a base sequence that can be hybridized (that is, complementary) with at least a part of the transcription product of the target gene or the targeted transcription product and can suppress the expression of the transcription product of the target gene or the expression level of the targeted transcription product primarily by means of the antisense effect.

The “target gene” or “targeted transcription product” whose expression is suppressed, modified, or altered by the antisense effect is not particularly limited. Examples of “target genes” include genes derived from organisms into which the double-stranded nucleic acid complex of the present disclosure has been introduced, genes whose expression is increased in various diseases, and the like.

In addition, the “transcription product of the target gene” is RNA that is transcribed from genomic DNA, e.g., mRNA or miRNA. mRNA is RNA that is transcribed from genomic DNA that encodes a protein.

In one embodiment of the present disclosure, the “transcription product” may be RNA that has not been subjected to base modification, RNA that has not been spliced, or the like. In an embodiment of this disclosure, the “targeted transcription product” may be noncoding RNA (i.e., ncRNA) such as miRNA rather than mRNA. Consequently, the “transcription product” may be any RNA that has been synthesized by a DNA-dependent RNA polymerase.

More generally, the “transcription product” may be any RNA synthesized by a DNA-dependent RNA polymerase.

In an embodiment of the present disclosure, a “targeted transcription product” may be, for example, apolipoprotein B (ApoB) mRNA, scavenger receptor B1 (SRB1) mRNA, metastasis associated lung adenocarcinoma transcript 1 (MALAT1) non-coding RNA, micro-RNA-122 (miR-122), β-secretase 1 (BACE1) mRNA, or PTEN (Phosphatase and Tensin Homolog Deleted from Chromosome 10) mRNA.

The base sequences of mouse and human ApoB mRNA are respectively indicated by SEQ ID NOS: 1 and 9 (however, the base sequence of mRNA is expressed as a base sequence of DNA). The base sequences of mouse and human SRB1 mRNA are respectively indicated by SEQ ID NOS: 2 and 10 (however, the base sequence of mRNA is expressed as a base sequence of DNA). The base sequences of mouse and human MALAT1 non-coding RNA are respectively indicated by SEQ ID NOS: 3 and 11 (however, the base sequence of mRNA is expressed as a base sequence of DNA). The base sequence of mouse miR-122 is indicated by SEQ ID NO: 4. The base sequence of human miR-122 is the same as that of mice. The base sequences of mouse and human BACE1 mRNA are respectively indicated by SEQ ID NOS: 5 and 12 (however, the base sequence of mRNA is expressed as a base sequence of DNA). The base sequences of mouse and human PTEN mRNA are respectively indicated by SEQ ID NOS: 6 and 13 (however, the base sequence of mRNA is expressed as a base sequence of DNA).

The base sequences of genes and transcription products can be obtained from a known database such as the NCBI (US National Center for Biotechnology Information) database, for example. The base sequences of micro-RNA can be obtained, for example, from the miRBase database (Kozomara A, Griffiths-Jones S. NAR 2014 42:D68-D73; Kozomara A, Griffiths-Jones S. NAR 2011 39:D152-D157; Griffiths-Jones S, Saini H K, van Dongen S, Enright A J. NAR 2008 36:D154-D158; Griffiths-Jones S, Grocock R J, van Dongen S, Bateman A, Enright A J. NAR 2006 34:D140-D144; Griffiths-Jones S. NAR 2004 32:D109-D111).

Note that as long as at least some of the nucleosides in at least one nucleic acid strand selected from the group consisting of the first nucleic acid strand and the second nucleic acid strand are bonded together by bonds including asymmetric phosphorus atoms, bonds not containing asymmetric phosphorus atoms may also be included.

One example of a method of stereoregulating asymmetric phosphorus atoms is a method of forming formulas D-1 and D-2 as an intermediate 28 by differentiating a compound (Rp) or (Sp)-20a-d expressed by the following formula A-1 or A-2, and then introducing an H-phosphonate structure with an S-configuration (Sp form) and an H-phosphonate structure with an R-configuration (Rp form) at any position.

In addition, stereoregulation of the asymmetric phosphorus atoms may be performed using formula A-3.

As starting materials, (Rp) or (Sp)-20a-d may be bonded with the hydroxy group at the 5′-position of the sugar structure at the terminal of an H-phosphonate-substituted nucleotide in the presence of an activator 21 to form the intermediate 28. Asymmetric auxiliary groups, base protecting groups, and R³ are then deprotected from the intermediate 28 so that an oligomer 29 is formed. Further, (Rp) or (Sp)-20a-d may be bonded with the hydroxy group at the 5′-position of the sugar structure at the terminal of the oligomer 29. This repetition allows the oligomeric chain to be elongated.

Note that the absolute configurations of asymmetric phosphorus atoms in phosphorothioate bonds can be regulated by subjecting the intermediate 28 to sulfurization.

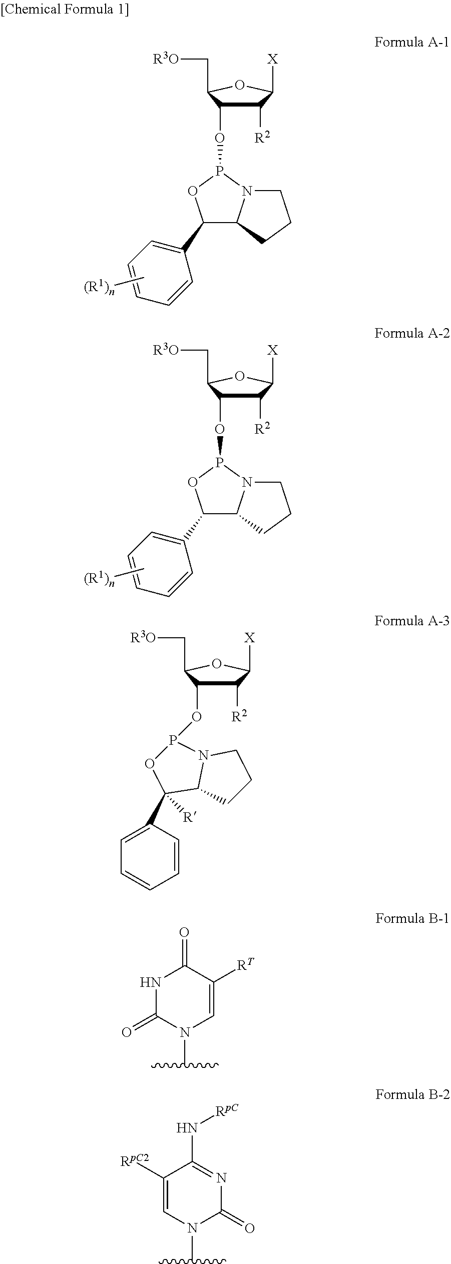

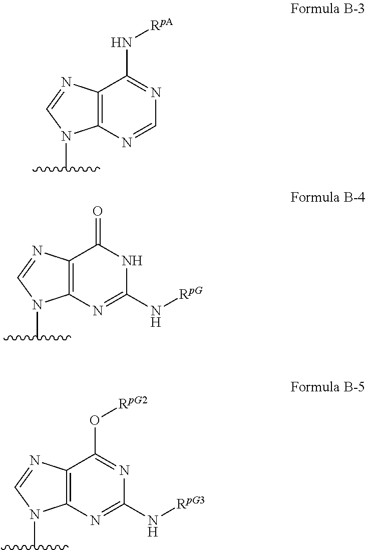

In formula A-1 or formula A-2, R¹ is an electron-donating group; n is an integer from 1 to 5; R² is a hydrogen atom, a halogen atom, or —OR^{o}; R^{o} is a hydrogen atom or a protecting group of an alkyl group or a hydroxy group, wherein the alkyl group may be bonded with a carbon atom at the 4′-position; R³ is a hydrogen atom or a protecting group of a hydroxy group; and X is a structure represented by any one of formulas B-1 to B-5.

In formula A-3, R², R³, and X are the same as R², R³, and X in formula A-1 and formula A-2, and R′ denotes an alkyl group.

In formulas B-1 to B-5, R^{T} is a hydrogen atom, an alkyl group, an alkenyl group, or an alkynyl group; R^{pC}, R^{pA}, and R^{pG} are protecting groups removed under acidic conditions; R^{pG2} is an alkyl group; R^{pG2} is a protecting group; R^{pG3} is a protecting group or a hydrogen atom removed under acidic conditions; and the wavy line indicates a bonding site with another structure.

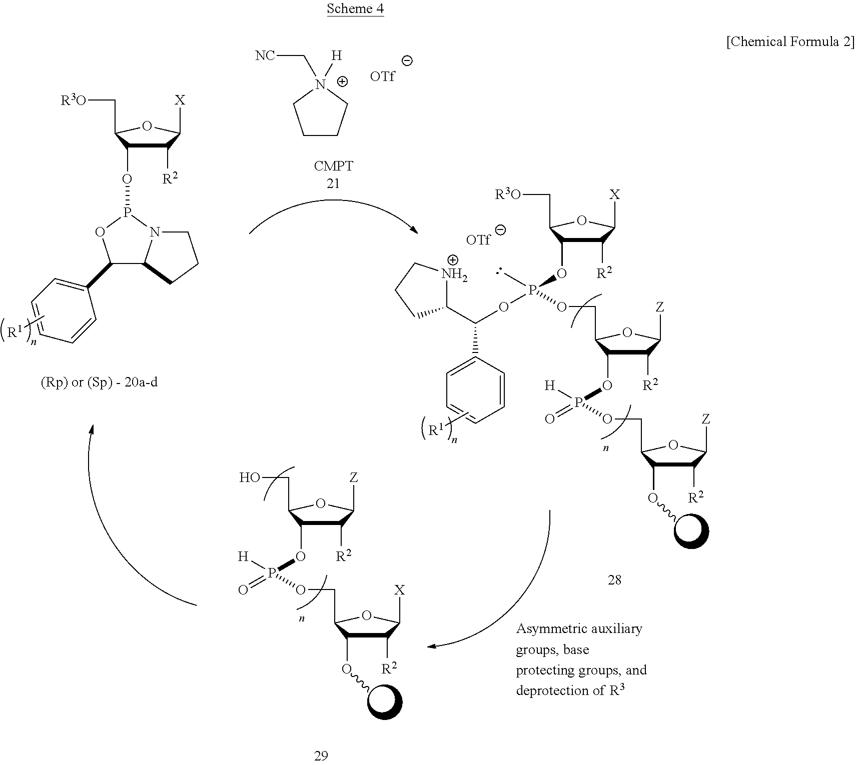

In scheme 4 above, R¹, n, R², R³, and X are each independently synonymous with R¹, n, R², R³, and X in formula A-1 or A-2, and this is also true for preferred modes.

The symbol n is an integer from 0 to 100, preferably an integer from 1 to 100, more preferably an integer from 9 to 100, and even more preferably an integer from 11 to 100.

In scheme 4 above, TfO (OTf) is a triflate anion, and Z is a structure expressed by any of formulas B-6 to B-9 below.

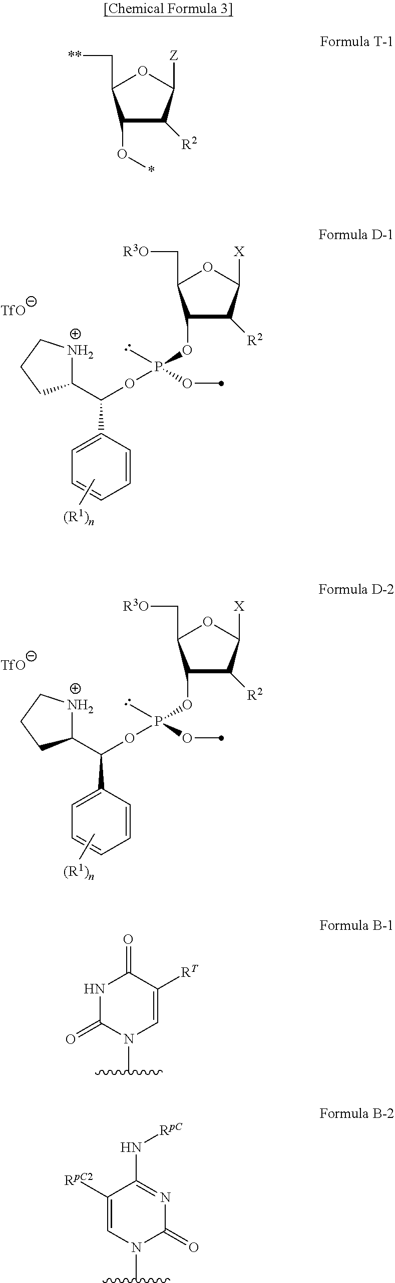

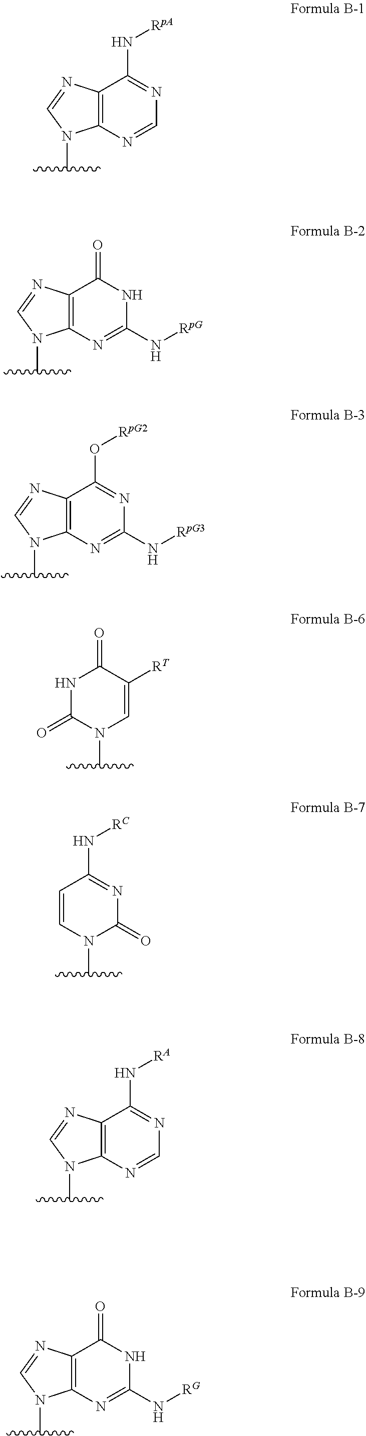

In formula T-1, R² is a hydrogen atom, a halogen atom, or —OR^{O}; R^{O} is a hydrogen atom or a protecting group of an alkyl group or a hydroxy group, wherein the alkyl group may be bonded with a carbon atom at the 4′-position; Z is a structure represented by any one of formulas B-6 to B-9; and * and ** indicate bonding sites with other structures.

In formula D-1 or D-2, R¹ is an electron-donating group; n is an integer from 1 to 5; R² is a hydrogen atom, a halogen atom, or —OR^{O} ; R^{O} is a hydrogen atom or a protecting group of an alkyl group or a hydroxy group, wherein the alkyl group may be bonded with a carbon atom at the 4′-position; R³ is a hydrogen atom or a protecting group of a hydroxy group; X is a structure represented by any one of formulas B-1 to B-5; TfO is a triflate anion; and indicates a bonding site with another structure.

In formulas B-1 to B-5, R^{T} is a hydrogen atom, an alkyl group, an alkenyl group, or an alkynyl group; R^{pC}, R^{pA}, and R^{pG} are protecting groups removed under acidic conditions; R^{pC2} is an alkyl group; R^{pG2} is a protecting group; R^{pG3} is a protecting group or a hydrogen atom removed under acidic conditions; and the wavy line indicates a bonding site with another structure.

In formulas B-6 to B-9, R^{T} is a hydrogen atom, an alkyl group, an alkenyl group, or an alkynyl group; R^{C}, R^{A}, and R^{G} are hydrogen atoms; and the wavy line indicates a bonding site with another structure.

For example, DNA, RNA, or the like in which the absolute configurations of asymmetric phosphorus atoms in phosphorothioate bonds are stereoregulated can be obtained by synthesizing in accordance with the scheme below.

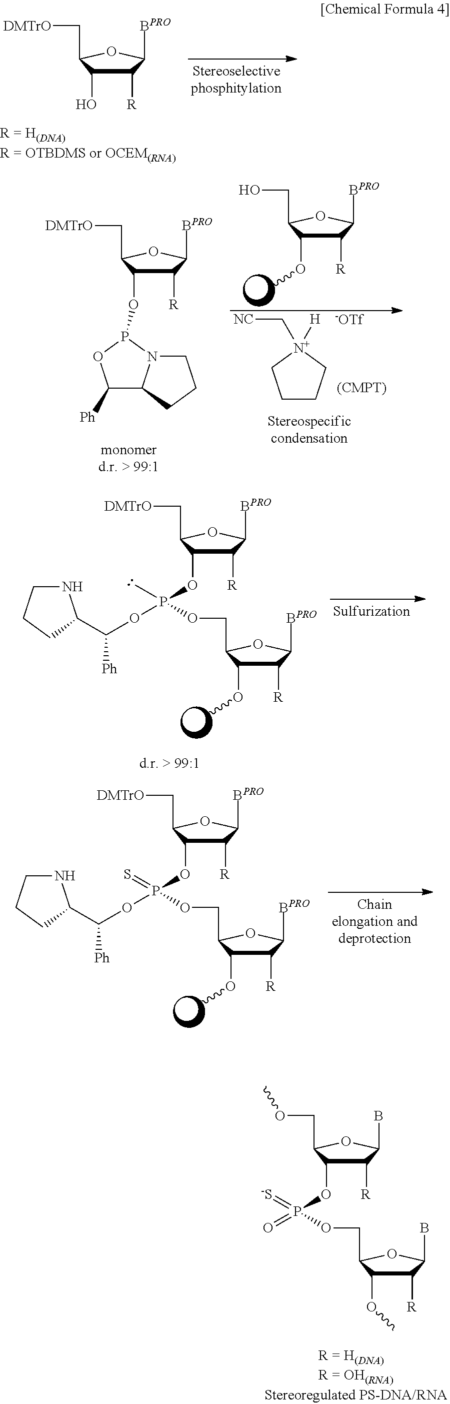

In an embodiment of the present disclosure, stereoregulation of asymmetric phosphorus atoms may be performed using compounds or methods described in paragraphs [0101] to [0177] of WO2014/010250.

The presence or absence of stereoregulation, in other words, differences in abundance of steric structure between a compound manufactured with stereoregulation and a compound manufactured without stereoregulation can be confirmed by well-known methods, e.g., nuclear magnetic resonance (NMR).

The bonds including asymmetric phosphorus atoms are not particularly limited, and examples thereof include phosphorothioate bonds, phosphotriester bonds, methylphosphonate bonds, methylthiophosphonate bonds, boranophosphate bonds, and phosphoroamidate bonds.

From the perspective of nuclease resistance, the bonds including asymmetric phosphorus atoms are preferably phosphorothioate bonds in at least one nucleic acid strand selected from the group consisting of the first nucleic acid strand and the second nucleic acid strand.

Note that a phosphorothioate bond refers to a bond between nucleosides in which non-bridged oxygen atoms of a phosphodiester bond have been substituted with sulfur atoms.

In addition, the stereoregulation of asymmetric phosphorus atoms in phosphorothioate bonds can be regulated by phosphorothioation of the intermediate 28 described above with a publicly known method.

### First Nucleic Acid Strand

The first nucleic acid strand includes at least one selected from the group consisting of natural nucleosides and non-natural nucleosides. The first nucleic acid strand according to the present disclosure may also contain both natural nucleosides and non-natural nucleosides.

In addition, from the perspective of suppressing the expression of a target gene, at least some of the nucleosides in the first nucleic acid strand are preferably bonded together by bonds including asymmetric phosphorus atoms, and the absolute configurations of the asymmetric phosphorus atoms are preferably regulated.

The term “natural nucleotide” in this specification includes deoxyribonucleotides observed in DNA and ribonucleotides observed in RNA.

“Deoxyribonucleotides” and “ribonucleotides” in this specification are also called “DNA nucleotides” and “RNA nucleotides,” respectively.

The term “natural nucleoside” in this specification includes deoxyribonucleosides contained in DNA and ribonucleosides contained in RNA.

“Deoxyribonucleosides” and “ribonucleosides” in this specification are also called “DNA nucleosides” and “RNA nucleosides,” respectively.

The term “non-natural nucleotide” in this specification refers to any nucleotide other than a natural nucleotide, and the term “non-natural nucleotide” includes modified nucleotides and nucleotide mimics.

Similarly, the term “non-natural nucleoside” in this specification refers to any nucleoside other than a natural nucleoside, and the term “non-natural nucleoside” includes modified nucleosides and nucleoside mimics.

The term “nucleoside mimic” in this specification includes sugars or sugars and bases as wells as structures which are unnecessary but are used to replace bonds at one or more positions of an oligomer compound. An “oligomer compound” refers to a hybridizable polymer of monomer units linked to at least one region of a nucleic acid molecule.

Examples of nucleoside mimics include morpholino, cyclohexenyl, cyclohexyl, tetrahydropyranyl, bicyclic or tricyclic sugar mimics such as nucleoside mimics having non-furanose units.

The term “nucleotide mimic” includes structures used to replace nucleotides and bonds at one or more position of an oligomer compound.

Non-natural oligonucleotides exhibit characteristics such as enhanced cell uptake, enhanced affinity to nucleic acid targets, and increased stability or increased inhibitory activity in the presence of nuclease, for example, in comparison to nucleic acid strands including natural oligonucleotides.

The term “modified nucleotide” in this specification refers to a nucleotide having any one or more modified sugar moiety, modified internucleoside bond, and modified nucleic acid base.

The term “modified nucleoside” in this specification refers to a nucleoside having at least one selected from the group consisting of a modified sugar moiety and a modified nucleic acid base.

The term “modified internucleoside bond” in this specification refers to an internucleoside bond having a substitution or any change from an internucleoside bond occurring in nature (that is, a phosphodiester bond), and this also includes bonds in which the absolute configurations of the asymmetric phosphorus atoms described above are regulated. A modified internucleoside bond is typically a bond with higher nuclease resistance than an internucleoside bond occurring in nature.

The positions of bonds containing stereoregulated asymmetric phosphorus atoms in the first nucleic acid strand are not particularly limited. The number of bonds containing stereoregulated asymmetric phosphorus atoms is not particularly limited. One or sequential bonds containing stereoregulated asymmetric phosphorus atoms, for example, may extend from at least one terminal selected from the group consisting of the 5′ terminal and the 3′ terminal of the first nucleic acid strand, and preferably 4 or 5 bonds containing stereoregulated asymmetric phosphorus atoms may extend from at least one terminal selected from the group consisting of the 5′ terminal and the 3′ terminal of the first nucleic acid strand.

In the double-stranded nucleic acid complex according to the present disclosure, the first nucleic acid preferably comprises:

two terminal regions including 2 to 10 consecutive nucleosides extending from the 5′ terminal and 3′ terminal of the first nucleic acid strand, respectively; and

a middle region that is positioned between the terminal regions and includes at least 4 nucleosides;

at least some of the nucleosides in at least one region selected from the group consisting of the terminal regions and the middle region being bonded together by bonds including asymmetric phosphorus atoms, and absolute configurations of the asymmetric phosphorus atoms being regulated.

The nucleosides in the terminal regions and the middle region are not particularly limited and may include at least one selected from the group consisting of natural nucleosides and non-natural nucleosides, or may include both natural nucleosides and non-natural nucleosides.

The nucleosides in the terminal regions may include both non-natural nucleosides and natural nucleosides, being a group consisting of at least one non-natural nucleoside.

The nucleosides in the middle region are not particularly limited and may include at least one selected from the group consisting of natural nucleosides and non-natural nucleosides, or may include both natural nucleosides and non-natural nucleosides.

When non-natural nucleosides are included in these regions, nucleosides that contain, for example, bridged nucleosides or 2′-O-MOE groups may be included.

Examples and preferred examples of natural nucleosides and non-natural nucleosides in the terminal regions and the middle region are the same as the natural nucleosides and non-natural nucleosides in the wing regions described below, and the preferred range is the same.

### Terminal Region

Preferably 2 to 10, more preferably 2 to 5 consecutive, nucleosides are contained in the two terminal regions in the first nucleic acid strand. Nucleosides contained in the terminal regions of the first nucleic acid strand are not particularly limited, and when the nucleosides contained in the terminal regions are non-natural nucleosides, regions containing the consecutive non-natural nucleosides are sometimes referred to as “wing regions.”

### Middle Region

Preferably at least 4, more preferably 4 to 12, nucleosides are contained in the middle region in the first nucleic acid strand.

Although nucleosides contained in the middle region of the first nucleic acid strand are not particularly limited, when the nucleosides contained in the middle region are natural nucleosides, regions containing 4 or more consecutive natural nucleotides are sometimes referred to as “gap regions.”

With the double-stranded nucleic acid complex of the present disclosure, at least some of the nucleosides in at least one region selected from the group consisting of the terminal regions and the middle region are bonded by bonds including asymmetric phosphorus atoms, and the absolute configuration of each asymmetric phosphorus atom is preferably regulated to be an S-configuration or an R-configuration.

Examples of combination units of absolute steric configurations of the asymmetric phosphorus atoms are: S configuration-S configuration-S configuration, S configuration-S configuration-R configuration, S configuration-R configuration-S configuration, S configuration-R configuration-R configuration, R configuration-S configuration-S configuration, R configuration-S configuration-R configuration, R configuration-R configuration-S configuration, and R configuration-R configuration-R configuration.

The terminal regions and the middle regions may contain a structure in which are repeated the above combination units of absolute configurations. For example, the terminal regions and middle region can contain structures in which the S configuration-S configuration-R configuration combination unit repeats.

The double-stranded nucleic acid complex of this disclosure also may contain nucleic acid structures that can be recognized by RNase H.

An example of a nucleic acid structure that is recognized by RNase H is a site that is cleaved by RNase H.

There are no particular limitations on the RNase H, provided that the RNase H can recognize double-stranded nucleic acid complexes of animals including humans.

With the double-stranded nucleic acid complex pertaining to the present disclosure, the first nucleic acid strand contains 4 or more consecutive deoxyribonucleosides, and the second nucleic acid strand described below contains 4 or more consecutive ribonucleosides, and the double-stranded nucleic acid complex may comprise a structure containing complementary base pairing between 4 or more consecutive deoxyribonucleosides and 4 or more consecutive ribonucleosides.

In addition, a bond between a non-natural nucleoside in the first nucleic acid strand and another nucleoside adjacent thereto is achieved by a bond including an asymmetric phosphorus atom, and the absolute configuration of the asymmetric phosphorus atom is regulated to an S-configuration or an R-configuration.

In the double-stranded nucleic acid complex pertaining to the present disclosure, the first nucleic acid strand may comprise a gap region containing 4 or more consecutive natural nucleosides and a wing region containing consecutive non-natural nucleosides extending from at least one region selected from the group consisting of the 5′ terminal and 3′ terminal of the gap region. The first nucleic acid strand of the double-stranded nucleic acid complex comprises wing regions and a gap region, and better antisense effects are thereby obtained. The first nucleic acid strand in the double-stranded nucleic acid complex of the present disclosure may be a “gapmer.”

The term “gapmer” in this specification denotes a nucleic acid strand comprising a gap region (DNA gap region) containing at least 4 consecutive deoxyribonucleosides, and a region containing non-natural nucleosides (5′ wing region and 3′ wing region) situated towards the 5′ terminal and towards the 3′ terminal from the gap region.

### Wing Region

The wing regions preferably include consecutive non-natural nucleosides extending from the 5′-terminal and the 3′-terminal of the gap region, respectively.

Note that in this specification, the wing region on the 5′-terminal side of the gap region may be called the “5′ wing region,” and the wing region on the 3′-terminal side of the gap region may be called the “3′ wing region.”

The base lengths (lengths) of the 5′ wing region and the 3′ wing region are each independent and may ordinarily be from 2 to 10 bases, from 2 to 7 bases, or from 2 to 5 bases.

The 5′ wing region and the 3′ wing region may also include natural nucleosides as long as they include consecutive non-natural nucleosides.

In the first nucleic acid strand, the non-natural nucleoside is preferably a sugar-modified nucleoside from the perspective of stability with respect to nuclease.

The term “sugar-modified nucleoside” in this specification refers to a modified nucleoside containing a modified sugar. In addition, a “modified sugar” refers to at least one selected from the group consisting of sugars having a substitution and any change from a natural sugar moiety (that is, a sugar portion observed in DNA (2′-H) or RNA (2′-OH)).

A sugar-modified nucleoside may impart to the nucleic acid strand enhanced stability with respect to nuclease, increase bond affinity, or a change in some other molecular biological characteristic.

A sugar-modified nucleoside includes a chemically modified ribofuranose ring moiety. Examples of chemically modified ribofuranose rings include but are not limited to the addition of substituents (including 5′ or 2′ substituents), the formation of bicyclic nucleic acids (bridged nucleic acids, BNAs) by bridging non-geminal ring atoms, the substitution of ribosyl ring oxygen atoms with S, N(R), or C(R¹)(R²) (R, R¹, and R² are each independently a hydrogen atom, an alkyl having from 1 to 12 carbon atoms, or a protecting group), and combinations thereof.

A sugar-modified nucleoside may include a 2′-modified sugar. A 2′-modified sugar may be a sugar including a 2′-O-methyl group.

The term “2′-modified sugar” in this specification refers to a furanosyl sugar modified at the 2′ position.

Examples of sugar-modified nucleosides include but are not limited to nucleosides including 5′-vinyl, 5′-methyl (R or S), 4′-S, 2′-F (2′-fluoro group), 2′-OCH₃ (2′-OMe group or 2′-O-methyl group), and 2′-O(CH₂)₂OCH₃(2′-O-MOE group) substituents.

A substituent at the 2′-position can be selected from the group consisting of allyl groups, amino groups, azide groups, thio groups, allyloxy groups, alkoxy groups having from 1 to 10 carbon atoms, —OCF₃, —O(CH₂)₂SCH₃, —O(CH₂)2—O—N(Rm)(Rn), and —O—CH₂—C(═O)—N(Rm)(Rn), and each Rm and Rn is independently a hydrogen atom or a substituted or unsubstituted alkyl having from 1 to 10 carbon atoms.

The term “2′-modified sugar” in this specification refers to a furanosyl sugar modified at the 2′ position.

Further examples of sugar-modified nucleosides include bicyclic nucleosides.

The term “bicyclic nucleoside” in this specification refers to a modified nucleoside including a bicyclic sugar moiety. A nucleic acid including a bicyclic sugar moiety is typically called a bridged nucleic acid (BNA).

In this specification, a nucleic acid including a bicyclic sugar moiety may also be called a “bridged nucleoside.”

A bicyclic sugar may be a sugar in which a carbon atom at the 2′-position and a carbon atom at the 4′-position are bridged by two or more atoms. Publically known and used sugars may be used as bicyclic sugars.

One subgroup of nucleic acid including a bicyclic sugar (BNA) can be described as having a 2′-position carbon atom and a 4′-position carbon atom which are bridged by 4′-(CH₂)ₚ—O-2′, 4′-(CH₂)ₚ—CH₂-2′, 4′-(CH₂)ₚ—S-2′, 4′ -(CH₂)ₚ—OCO-2′, and 4′-(CH₂)ₙ-N(R₃)—O—(CH₂)ₘ-2′ (in the formulas, p, m, and n are respectively integers from 1 to 4, from 0 to 2, and from 1 to 3; R₃ is a hydrogen atom, an alkyl group, an alkenyl group, a cycloalkyl group, an aryl group, an aralkyl group, an acyl group, a sulfonyl group, and a unit substituent (a fluorescent or chemiluminescent labeled molecule, a functional group having nucleic acid cleaving activity, a localized signal peptide in a cell or in a nucleus, or the like).

Further, with regard to the bridged nucleic acid (BNA) of a specific embodiment, in the OR₂ substituent of the carbon atom at the 3′-position and the OR₁ substituent of the carbon atom at the 5′-position, R₁ and R₂ are typically hydrogen atoms, but they may be the same as or different than one another and may also be a protecting group of a hydroxyl group for nucleic acid synthesis, an alkyl group, an alkenyl group, a cycloalkyl group, an aryl group, an aralkyl group, an acyl group, a sulfonyl group, a silyl group, a phosphoric acid group, a phosphoric acid group protected by a protecting group for nucleic acid synthesis, or a group represented by —P(R₄)R₅ (R₄ and R₅ may be the same as or different than one another and may each be a hydroxyl group, a hydroxyl group protected by a protecting group for nucleic acid synthesis, a mercapto group, a mercapto group protected by a protecting group for nucleic acid synthesis, an amino group, an alkoxy group having from 1 to 5 carbon atoms, an alkylthio group having from 1 to 5 carbon atoms, a cyanoalkoxy group having from 1 to 6 carbon atoms, or an amino group substituted with an alkyl group having from 1 to 5 carbon atoms).

Such a bridged nucleic acid (BNA) is not particularly limited. Examples of publicly known and used bridged nucleic acids (BNAs) include methyleneoxy (4′-CH₂—O-2′) BNA (LNA (Locked Nucleic Acid (registered trademark)); also known as 2′,4′-BNA), α-L-methyleneoxy (4′-CH₂—O-2′) BNA or β-D-methyleneoxy (4′-CH₂—O-2′) BNA, ethyleneoxy (4′-(CH₂)₂—O-2′) BNA (also known as ENA), β-D-thio (4′-CH₂—S-2′) BNA, aminooxy(4′-CH₂—O—N(R₃)-2′) BNA, oxyamino (4′-CH₂—N(R₃)—O-2′) BNA (also known as (2′,4′-BNA^{NC}), 2′,4′-BNA^{COC}, 3′-amino-2′,4′-BNA, 5′-methyl BNA, (4′-CH(CH₃)—O-2′) BNA (also known as cEt BNA), (4′-CH(CH₂OCH₃)—O-2′) BNA (also known as cMOE BNA), and amide BNA (4′-C(O)—N(R)-2′) BNA (R═H or Me) (also known as AmNA).

In this specification, a bridged nucleoside having a methyleneoxy (4′-CH₂—O-2′) bridge (bicyclic nucleoside) may also be called an “LNA nucleoside.”

The modified sugar may be prepared by a publically known and used method.

In a modified sugar nucleotide, the nucleic acid base moiety (natural, modified, or a combination thereof) may be maintained for hybridization with the target nucleic acid.

In the first nucleic acid strand, the sugar-modified nucleoside preferably contains a bridged nucleoside and more preferably contains an LNA nucleoside.

A bridged nucleoside may include a modified nucleic acid base.

In this specification, a “modified nucleic acid base” or a “modified base” refers to a so-called nucleic acid base other than adenine, cytosine, guanine, thymine, or uracil. An “unmodified nucleic acid base” or an “unmodified base” (natural nucleic acid base) refers to adenine (A) and guanine (G), which are purine bases, and thymine (T), cytosine (C), and uracil (U), which are pyrimidine bases.

Examples of modified nucleic acid bases include but are not limited to 5-methylcycosine, 5-fluorocytosine, 5-bromocytosine, 5-iodocytosine, or N4-methylcytosine; 5-fluorouracil, 5-bromouracil, or 5-iodouracil; 2-thiothymine; N6-methyladenine or 8-bromoadenine; and N2-methylguanine or 8-bromoguanine.

From the perspective of the antisense effect, bonds between bridged nucleosides are preferably bonds including asymmetric phosphorus atoms in which the absolute configurations of the asymmetric phosphorus atoms are regulated to the R configuration (Rp).

In addition, from the perspective of anti-nuclease activity, bonds between bridged nucleosides are preferably phosphorothioate bonds.

### Gap Region

The gap region is positioned between the 3′ wing region and the 5′ wing region and includes four or more consecutive natural nucleosides.

The gap region is not particularly limited as long as it includes four or more consecutive natural nucleosides, and the gap region may include non-natural nucleosides such as nucleosides containing a 2′-O-MOE group, for example.

Note that the specific examples of non-natural nucleosides are synonymous with the non-natural nucleosides in the wing region, and the preferable range is also the same.

The base length of the gap region is preferably from 4 to 20 bases, more preferably from 4 to 15 bases, and even more preferably from 4 to 10 bases.

The natural nucleosides in the gap region are preferably deoxyribonucleosides or ribonucleosides and more preferably deoxyribonucleosides.

From the perspective of the antisense effect, bonds between natural nucleosides are preferably bonds including asymmetric phosphorus atoms in which the absolute configuration of each asymmetric phosphorus atom is regulated to the S configuration (Sp) or the R configuration (Rp), or bonds including asymmetric phosphorus atoms in which the absolute configuration of each asymmetric phosphorus atom is not regulated (also called “non-stereoregulated” hereafter), and the bonds are more preferably bonds including asymmetric phosphorus atoms in which the absolute configuration of each asymmetric phosphorus atom is regulated to the R configuration or bonds including asymmetric phosphorus atoms in which the absolute configuration of each asymmetric phosphorus atom is not stereoregulated.

In addition, from the perspective of anti-nuclease activity, bonds between deoxyribonucleosides are preferably phosphorothioate bonds.

From the perspective of the antisense effect, the base length of the first nucleic acid strand is preferably from 8 to 30 bases, more preferably from 8 to 20 bases, and even more preferably from 10 to 15 bases.

For example, when the base length of the first nucleic acid strand is 13 bases, the bond between two nucleosides may be in the R configuration (Rp) from the 5′-side, and the next 7 bonds may be a mixture of the R configuration (Rp) and the S configuration (Sp) (non-stereoregulated), while the following 3 on the 3′-side may be in the R configuration (Rp).

The first nucleic acid strand may further contain at least one nucleic acid selected from the group consisting of a peptide nucleic acid and a morpholino nucleic acid.

Each of a peptide nucleic acid and a morpholino nucleic acid (—N(H)—C(═O)—O— or other morpholino bonded by a non-phosphodiester bond) is one of the nucleotide mimics described above.

A peptide nucleic acid (PNA) is a nucleotide mimic having a main strand in which N-(2-aminoethyl) glycine is bonded with an amide bond rather than a sugar.

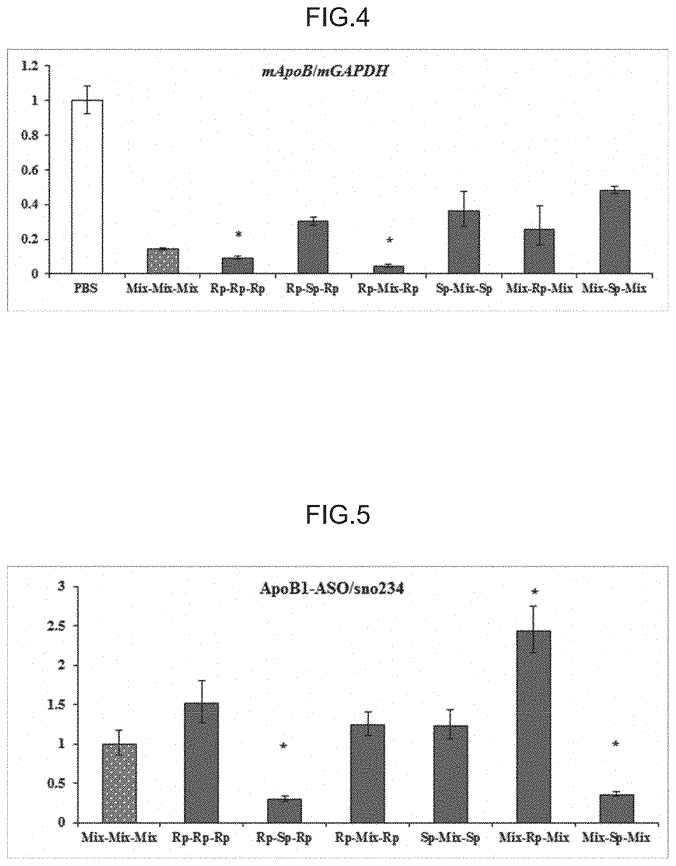

The structure of a morpholino nucleic acid is illustrated in FIG. 4.

In the double-stranded nucleic acid complex according to the present disclosure, the first nucleic acid strand may be a “mixmer.”

In this specification, a “mixmer” refers to a nucleic acid strand which contains interchangeable natural nucleosides (meaning at least one of deoxyribonucleosides and ribonucleosides) and non-natural nucleosides of periodic or random segment length and does not have 4 or more consecutive deoxyribonucleosides or 4 or more consecutive ribonucleosides.

A mixmer in which the non-natural nucleosides are bridged nucleosides and the natural nucleosides are deoxyribonucleosides is sometimes called a “BNA/DNA mixmer.”

A mixmer in which the non-natural nucleosides are bridged nucleosides and the natural nucleosides are ribonucleosides is sometimes called a “BNA/RNA mixmer.”

A mixmer does not necessarily need to be limited so as to contain only two types of nucleosides. A mixmer may contain any number of types of nucleosides, regardless of whether they are natural or modified nucleosides or nucleoside mimics For example, a mixmer may have 1 or 2 consecutive deoxyribonucleosides separated by bridged nucleosides (for example, LNA nucleosides). Bridged nucleosides may contain modified nucleic acid bases (for example, 5-methylcytosine).

### Second Nucleic Acid Strand

In the double-stranded nucleic acid complex according to the present disclosure, the second nucleic acid strand includes a complementary region having a base sequence complementary to the first nucleic acid strand. Therefore, in the double-stranded nucleic acid complex, the first nucleic acid strand is annealed to the complementary region in the second nucleic acid strand.

The complementary region in the second nucleic acid strand may include natural nucleosides, non-natural nucleosides, or both.

Note that the natural nucleosides and non-natural nucleosides included in the second nucleic acid strand are the same as the natural nucleosides and non-natural nucleosides included in the first nucleic acid strand.

From the perspective of achieving an excellent antisense effect, in the double-stranded nucleic acid complex according to the present disclosure, the first nucleic acid strand preferably comprises the wing region and gap region described above, and when the gap region includes deoxyribonucleosides, the complementary region in the second nucleic acid strand preferably includes nucleosides, more preferably includes consecutive ribonucleosides, and even more preferably contains at least 3 and particularly preferably at least 4 or 5 consecutive ribonucleosides.

When there are such consecutive ribonucleosides in the second nucleic acid strand, a double strand can be formed with the DNA gap region of the first nucleic acid strand. This double strand is recognized by RNase H and can promote the cleaving of the second nucleic acid strand by RNase H.

The complementary region in the second nucleic acid strand may also be a region which does not include at least two consecutive ribonucleosides.

In addition, the double-stranded nucleic acid complex according to the present disclosure is a double-stranded nucleic acid complex comprising a first nucleic acid strand and a second nucleic acid strand bonded to each other, the second nucleic acid strand including a complementary region having a base sequence complementary to the first nucleic acid strand; the first nucleic acid strand including: a gap region including four or more consecutive deoxyribonucleosides; and wing regions including consecutive bridged nucleosides extending from the 5′-terminal and the 3-terminal of the gap region, respectively;

At least some of the nucleosides in the first nucleic acid strand being bonded together by bonds including asymmetric phosphorus atoms, and absolute configurations of the asymmetric phosphorus atoms being regulated; and the second nucleic acid strand including ribonucleosides.

### Functional Moiety

From the perspective of achieving excellent delivery to a target site, the second nucleic acid strand may further include at least one functional moiety bonded with a polynucleotide.

The functional moiety may be linked to the 5′-terminal of the second nucleic acid strand or may be linked to the 3′-terminal and linked to the nucleotides inside the polynucleotide.

In the second nucleic acid strand, the number of functional moieties is not particularly limited and may also be 2 or greater. When the second nucleic acid strand includes two or more functional moieties, the two or more functional moieties are not particularly limited and may be linked to a plurality of positions of the polynucleotide or may be linked as a group to a single position of the polynucleotide.

The bond between the second nucleic acid strand and the functional moiety may be a direct bond or may be an indirect bond mediated by another material.

In an embodiment of the present disclosure, the functional moiety is preferably directly bonded to the second nucleic acid strand via covalent bonding, ionic bonding, hydrogen bonding, or the like, and from the perspective that more stable bonding may be achieved, covalent bonding is more preferable.

The functional moiety may be bonded to the second nucleic acid strand via a cleavable linker moiety (linking group). For example, the functional moiety may be linked by a disulfide bond.

The structure of the functional moiety is not particularly limited as long as any one of a labeling function, a purification function, and a targeted delivery function is imparted to at least one selected from the group consisting of the double-stranded nucleic acid complex and the second nucleic acid strand to which the functional moiety is bonded.

The functional moiety in the second nucleic acid strand preferably has at least one function selected from the group consisting of a labeling function, a purification function, and a targeted delivery function.

Examples of moieties which impart a labeling function include compounds such as fluorescent proteins and luciferase. Examples of moieties which impart a purification function include compounds such as biotin, avidin, a His-tag peptide, a GST-tag peptide, and a FLAG-tag peptide.

In an embodiment of the present disclosure, the functional moiety fulfills a role of enhancing delivery to a cell or a cellular nucleus. For example, when a specific peptide tag is conjugated with an oligonucleotide, the cell uptake of the oligonucleotide is enhanced. Examples include arginine-rich peptide P007 and B-peptide disclosed in Hai Fang Yin et al., Human Molecular Genetics, Vol. 17(24), 3909-3918 (2008) and the references thereof. Intranuclear transfer can be enhanced conjugating a portion such as m3G-CAP (see Pedro M.D. Moreno et al., Nucleic Acids Res., Vol. 37, 1925-1935 (2009)) with an oligonucleotide.

Further, from the perspective of delivering the double-stranded nucleic acid complex (or first nucleic acid strand) according to the present disclosure to a target site or a target region in the body with high specificity and high efficiency so as to effectively suppress the expression of a targeted transcription product (for example, a target gene) due to related nucleic acids, an active molecule which delivers the double-stranded nucleic acid complex of an embodiment of the present disclosure to a “target site” in the body is preferably bonded to the second nucleic acid strand as a functional moiety.

When the functional moiety has a “targeted delivery function,” the functional moiety is preferably at least one molecule species selected from a lipid, an antibody, a peptide, and a protein from the perspective of being able to deliver the double-stranded nucleic acid complex according to the present disclosure to the liver or the like with high specificity and high efficiency.

Examples of lipids include lipids such as cholesterol and fatty acids (for example, vitamin E (tocopherol, tocotrienol), vitamin A, and vitamin D); lipid-soluble vitamins such as vitamin K (for example, acylcarnitine); intermediate metabolites such as acyl-CoA; glycolipids, glycerides, and derivatives thereof.

Of these, from the perspective of achieving higher safety, the lipid is preferably at least one selected from cholesterol, tocopherol, and tocotrienol.

Further, from the perspective of being able to deliver the double-stranded nucleic acid complex according to the present disclosure to the brain with specificity and high efficiency, the functional moiety may also be a cholesterol or an analog thereof, a tocopherol or an analog thereof, or a sugar (for example, glucose and sucrose).

The second nucleic acid strand may further include an overhang region positioned on at least one terminal selected from the group consisting of the 5′-terminal and the 3′-terminal of the complementary region described above. The overhang region is preferably a single-strand region.

When the first nucleic acid strand and the second nucleic acid strand are annealed to form a double-stranded structure, the “overhang region” in this specification indicates at least one region selected from the group consisting of a nucleotide region in the second nucleic acid strand in which the 5′-terminal of the second nucleic acid strand extends beyond the 3′-terminal of the first nucleic acid strand, and a nucleotide region in the second nucleic acid strand in which the 3′-terminal of the second nucleic acid strand extends beyond the 5′-terminal of the first nucleic acid strand. That is, the overhang region is a nucleotide region in the second nucleic acid strand which projects from the double-stranded structure and is adjacent to the complementary region described above.

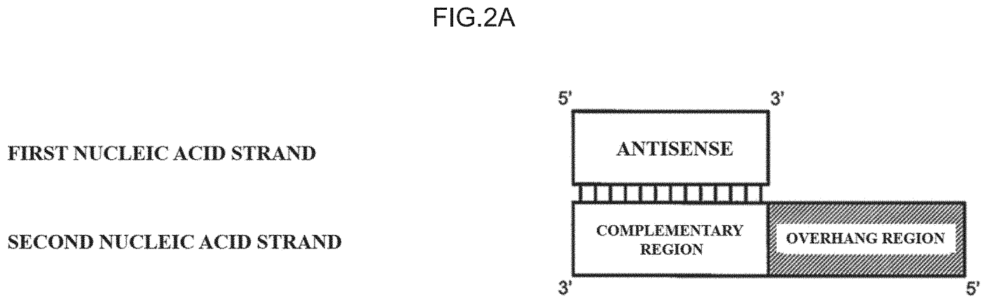

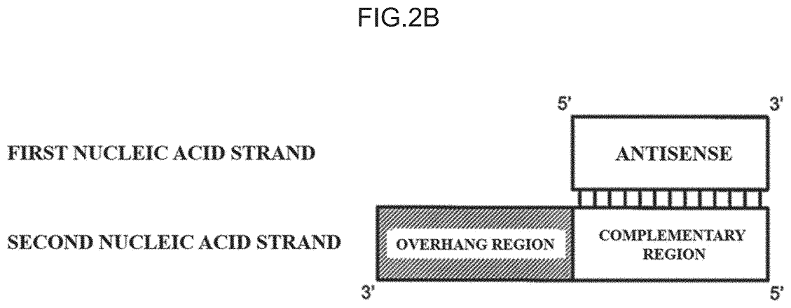

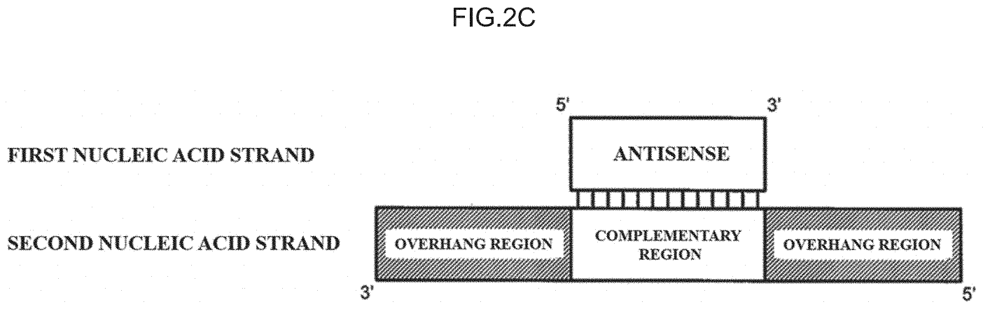

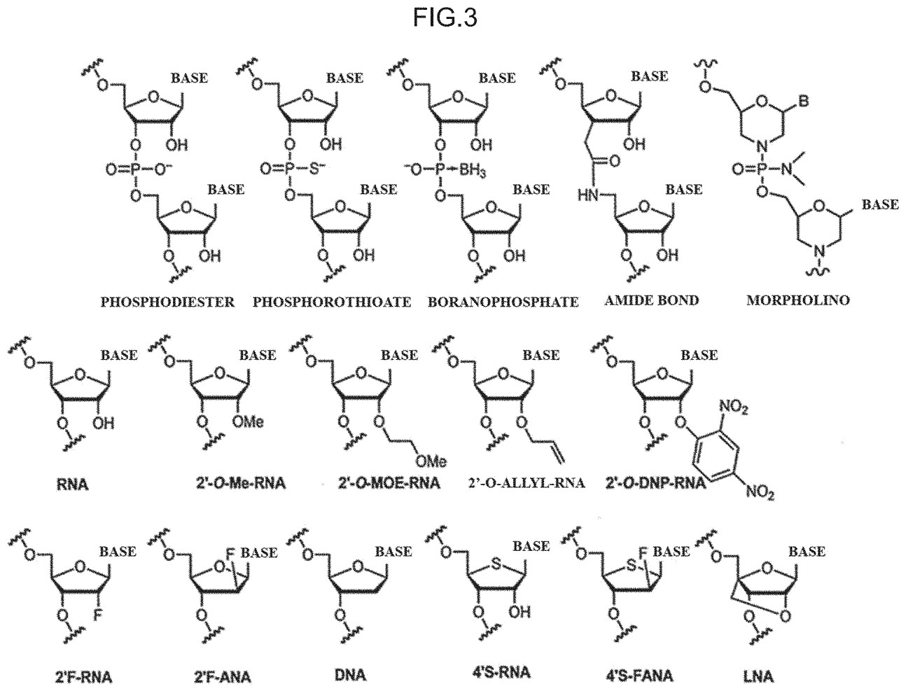

In the second nucleic acid strand, the position of the overhang region is not particularly limited, and the overhang region may be positioned on the 5′-terminal side (FIG. 2A) or on the 3′-terminal side (FIG. 2B) of the complementary region. The overhang region in the second nucleic acid strand may also be positioned on the 5′-terminal side and the 3′-terminal side of the complementary region (FIG. 2C).

The overhang region may be a single region on the 5′-terminal side or the 3′-terminal side of the complementary region, or it may be two regions on the 5′-terminal side and the 3′-terminal side of the complementary region.

The base length of the overhang region is preferably at least 1 base, and more preferably at least 9 bases. For example, the base length may be from 1 to 30 bases, preferably from 9 to 17 bases, and even more preferably from 11 to 15 bases.

When there are two overhang regions in the second nucleic acid strand, the lengths of the overhang regions may be the same as or different than one another.

The base length of the second nucleic acid strand is not particularly limited, but from the perspective of synthesis cost or delivery efficiency, the base length is preferably not greater than 40 bases, more preferably from 18 to 30 bases, and even more preferably from 21 to 28 bases.

Note that when the second nucleic acid strand includes an overhang region, the base length of the second nucleic acid strand refers to the total base length of the complementary region and the overhang region.

A bond between a nucleoside in the second nucleic acid strand including the overhang region and another nucleoside adjacent thereto is achieved by a bond including an asymmetric phosphorus atom, and the absolute configuration of the asymmetric phosphorus atom may be regulated to Sp or Rp.

Note that bonds including asymmetric phosphorus atoms are synonymous with the bonds including asymmetric phosphorus atoms described above.

The overhang region may include natural nucleosides, non-natural nucleosides, or both.

The overhang region in the second nucleic acid strand is preferably not an oligonucleotide region for treatment.

Examples of oligonucleotides for treatment include antisense oligonucleotides, microRNA inhibitors (antimiR), splice-switching oligonucleotides, single-stranded siRNA, microRNA, and pre-microRNA.

Since the overhang region in the second nucleic acid strand does not have an oligonucleotide for treatment such as that described above, it has essentially no capacity to hybridize the transcription product in the cell, and it is therefore unlikely to affect gene expression.

At least one nucleoside (specifically, from 1 to 3 nucleosides) from a terminal of the complementary region that is not bonded to the overhang region (also called the “free terminal of the complementary region” hereafter) is preferably a sugar-modified nucleoside.

Further, at least one nucleoside (for example, at least 2 or at least 3; specifically, from 1 to 3) from the bonding terminal of the overhang region is a modified nucleoside.

Note that the sugar-modified nucleoside is synonymous with the sugar-modified nucleoside in the first nucleic acid strand.

The overhang region may include sugar-modified nucleosides and may have a base length of from 9 to 12 bases. The overhang region may also contain no sugar-modified nucleosides, and the base length of the overhang region may be from 9 to 17 bases.

The double-stranded nucleic acid complex according to the present disclosure is produced by stereoregulating at least one selected from the group consisting of the first nucleic acid strand and the second nucleic acid strand using the method described above, for example. The other may be produced by the method described above or may be produced using an automatic nucleic acid synthesizer based on the operations described below.

The double-stranded nucleic acid complex may also be obtained by annealing the respectively produced first nucleic acid strand and second nucleic acid strand.

For example, the nucleic acids according to certain embodiments of the present disclosure can be produced by designing the base sequences of each of the nucleic acids based on information indicating the base sequence of the targeted transcription product (or, in some examples, the base sequence of the target gene), synthesizing a nucleic acid using a commercially available automatic nucleic acid synthesizer (product of Applied Biosystems, Inc., product of Beckman Coulter Inc., or the like), and then purifying the resulting oligonucleotide using a reverse phase column or the like.

Nucleic acids produced with this method are mixed in an appropriate buffer solution and denatured for several minutes (e.g., 5 min) at about 90° C. to 98° C. The nucleic acids are then annealed for about 1 to 8 hours at about 30° C. to 70° C., and the double-stranded nucleic acid complex according to the present disclosure can be produced in this way.

The production of the double-stranded nucleic acid complex is not limited to such time and temperature protocols.

The conditions suitable for promoting the annealing of a double strand are well known in this technical field. Further, a nucleic acid complex to which a functional moiety has been bonded can be produced by performing the synthesis, purification, and annealing described above using a nucleic acid species to which a functional moiety has been bonded in advance.

The method for linking functional moieties to nucleic acids can be implemented in accordance with a publically known and used method. The nucleic acid strands constituting the double-stranded nucleic acid complex may be obtained by designating the base sequence and the modification site or type.

The double-stranded nucleic acid complex according to the present disclosure is delivered efficiently into the body due in part to such changes in the bonds with serum proteins, which makes it possible to suppress target gene expression or the level of the targeted transcription product using the antisense effect. Accordingly, the double-stranded nucleic acid complex according to the present disclosure may be used to suppress target gene expression or the level of the targeted transcription product.

### Pharmaceutical composition

The pharmaceutical composition according to the present disclosure contains the double-stranded nucleic acid complex described above and a pharmaceutically acceptable carrier.

A composition containing the nucleic acid complex described above as an active ingredient for suppressing target gene expression or the expression level of the targeted transcription product by the antisense effect is also provided.

In this specification, the term “expression level of the targeted transcription product” is used interchangeably with the “amount of the targeted transcription product expressed.”

The pharmaceutical composition according to the present disclosure can be formulated by a known formulation method. For example, this composition can be used orally or non-orally in the form of capsules, tablets, pills, liquids, powders, granules, fine granules, film-coating agents, pellets, troches, sublingual agents, peptizers, buccal preparations, pastes, syrups, suspensions, elixirs, emulsions, coating agents, ointments, plasters, cataplasms, transdermal preparations, lotions, inhalers, aerosols, eye drops, injections, and suppositories.

In regard to the formulation of these preparations, pharmacologically acceptable carriers or carries acceptable as food and drink—specifically, sterilized water, physiological saline, vegetable oils, solvents, bases, emulsifiers, suspending agents, surfactants, pH adjusting agents, stabilizers, flavors, fragrances, excipients, vehicles, antiseptics, binders, diluents, isotonizing agents, soothing agents, extending agents, disintegrants, buffering agents, coating agents, lubricants, colorants, sweetening agents, thickening agents, corrigents, dissolution aids, and other additives—can be appropriately incorporated into the preparations.

The administration method of the pharmaceutical composition according to the present disclosure is not particularly limited, and examples include oral administration or non-oral administration, and more specifically, intravenous administration, intraventricular administration, intrathecal administration, subcutaneous administration, intraarterial administration, intraperitoneal administration, intracutaneous administration, intratracheobronchial administration, rectal administration, intraocular administration, transnasal administration, intramuscular administration, and administration by transfusion.

Note that subcutaneous administration may be advantageous from the perspective of ease of administration in comparison to intravenous administration.

In an embodiment of the present disclosure, when used in subcutaneous administration, the double-stranded nucleic acid complex according to the present disclosure may not include bonds with lipids such as vitamin E (tocopherol, tocotrienol) and cholesterol.

The use and method of the pharmaceutical composition according to the present disclosure are not particularly limited and may be, for example, a use or method of administering the pharmaceutical composition into a cell to alter the function of a transcription product in a cell, a use or method of changing the expression level of a protein in a cell, or a use or method of changing the protein structure in a cell.

The types of cells into which the pharmaceutical composition according to the present disclosure may be administered are not particularly limited. Examples of types of cells include immune cells, epithelial cells, vascular endothelial cells, and mesenchymal cells.

The pharmaceutical composition according to the present disclosure can be used in animals including humans as subjects. There are no particular limitations on animals excluding humans, and various domestic animals, domestic fowl, pets, experimental animals, and the like may be used as subjects in some embodiments.

When the pharmaceutical composition according to the present disclosure is administered or ingested, the dose or the amount ingested may be appropriately selected in accordance with the age, body weight, symptoms and health of the subject, the type of the composition (pharmaceutical product, food and drink, or the like), and the like.

The effective daily amount of ingestion per kilogram of body weight of the pharmaceutical composition according to the present disclosure may be, for example, from 0.0000001 mg/kg/day to 1,000,000 mg/kg/day, from 0.00001 mg/kg/day to 10,000 mg/kg/day, or from 0.001 mg/kg/day to 100 mg/kg/day of the nucleic acid complex.

The pharmaceutical composition according to the present disclosure may be used, for example, to treat or prevent diseases associated with genetic mutations or increased expression of target genes (for example, metabolic disorders, tumors, infections, and the like).

The pharmaceutical composition according to the present disclosure may also be a pharmaceutical composition for intraventricular administration or intrathecal administration to treat or prevent central nervous system disorders.

In one embodiment, the double-stranded nucleic acid complex used in intraventricular or intrathecal administration may be one that does not include bonds with lipids such as vitamin E (tocopherol, tocotrienol) and cholesterol.

The method may also be a method of administering the pharmaceutical composition according to the present disclosure into a cell to treat a central nervous system disorder.

Examples of central nervous system disorders include but are not limited to Huntington's disease, Alzheimer's disease, Parkinson's disease, amyotrophic lateral sclerosis (ALS), and brain tumors.

### EXAMPLES

The present disclosure will be described in further detail hereinafter using embodiments. However, the present disclosure is not limited to these embodiments.

The sequences of the oligonucleotides used in the following embodiments are shown collectively in Table 1.

TABLE 1

| Oligonucleotide | | | Examples/ |
|---|---|---|---|
| name | Sequence | SEQ ID NO. | Comparative Example |
| LNA-ASO 1-7 | 5′-G(L)*C(L)*a*t*t*g*g*t*a*t*T(L)*C(L)* | 7 | Examples 1 to 6, |
| (13 mer) | A(L)-3′ | | Comparative Example 1 |
| TOC-cRNA | 5′-TOC-U(M)G(M)A(M)AUACCAAUG(M)*C(M)-3′ | 8 | Examples 1 to 6, |
| | | | Comparative Example 1 |

In Table 1, a capital letter followed by “(L)” represents LNA (e.g., C(L) represents 5-methylcytosine LNA), a lowercase letter represents DNA, a capital letter represents RNA, a capital letter followed by “(M)” represents 2′-O-Me RNA, * represents phosphorothioate, and Toc represents tocopherol.

### Example 1

### Production of Double-Stranded Nucleic Acid Complex

As a first nucleic acid strand, an antisense oligonucleotide (ASO) was prepared (LNA/DNA gapmer) in which the LNA nucleosides included in each wing region formed an oligomer by mutual phosphorothioate bonding, and in which the gap region was DNA. Specifically, a single-stranded ASO (LNA-ASO1) was prepared in which the absolute configurations of the asymmetric phosphorus atoms in the wing region and the gap region were regulated to the R configuration, in accordance with the asymmetric phosphorus atom stereoregulation method described above and the method described in WO 2014/010250.

The oligonucleotide having asymmetric phosphorus atoms regulated to the R configuration (Rp) was synthesized by stereoregulating the asymmetric phosphorus atoms with the method described above.

As a second nucleic acid strand, Toc-cRNA, which is a nucleic acid strand having a base sequence complementary to LNA-ASO1 and having tocopherol bonded with the 5′-terminal thereof, was prepared.

The Toc-cRNA that was used was commissioned to Gene Design, Inc. for synthesis.

Note that the LNA/DNA gapmer is a 13-mer LNA/DNA gapmer that is complementary to positions 10136 to 10148 of the mRNA (sequence no. 1) of mouse apolipoprotein B.

Note that the LNA/DNA gapmer includes 2 LNA nucleosides in the 5′-terminal wing region, 3 LNA nucleosides in the 3′-terminal wing region, and 8 DNA nucleosides in the gap region between the 5′-terminal wing region and the 3′-terminal wing region.

After the LNA-ASO1 described above was dissolved in a phosphate buffer solution (PBS) (pH 7.4) so that the concentration was 200 μmol/L, the solution was mixed with an equimolar amount of Toc-cRNA to prepare a mixed solution.

This mixed solution was heated for 5 minutes at 95° C., cooled to 37° C., and then kept at this temperature for one hour. As a result of this treatment, the first nucleic acid strand and the second nucleic acid strand were annealed to prepare a double-stranded nucleic acid complex. The double-stranded nucleic acid complex was stored at 4° C. or on ice until use.

### Evaluation

### Antisense Effect According to in vivo Experiment

The double-stranded nucleic acid complex prepared above was intravenously injected into 4-week-old female ICR mice weighing from 20 to 25 g through the caudal veins in an amount of 0.75 mg/kg (3 mice per group were used).

In addition, mice which were injected with only PBS instead of the double-stranded nucleic acid complex were also prepared as a negative control group.

After 72 hours passed following intravenous injection, the mice were perfused with PBS, and the mice were then dissected to extract the livers. Next, RNA was extracted in accordance with the protocol using a small RNA extraction reagent (product name: ISOGEN II, made by Nippon Gene Co., Ltd.).

Using the extracted RNA, cDNA was synthesized in accordance with the protocol by utilizing a cDNA synthesis kit (product name: Transcriptor Universal cDNA Master, DNase, made by Roche Diagnostics Co., Ltd.).

Using the synthesized cDNA as a template, quantitative RT-PCR was carried out with the TaqMan method using primers designed and manufactured by Thermo Fisher Scientific based on a variety of number of genes.

The amplification conditions for quantitative RT-PCR were one cycle of 15 seconds at 95° C., 30 seconds at 60° C., and 1 second at 72° C., and this was repeated for 40 cycles.

Based on the results of quantitative RT-PCR obtained in this way, the amount of apolipoprotein B (ApoB) expressed/the amount of GAPDH (internal reference standard) expressed were respectively calculated.

In addition, the results of each of the groups were compared, and student's T-test was performed after one-way analysis of variance (ANOVA). Multiple comparisons were made with the Bonferroni method. The results are shown in FIG. 4.

The data in FIG. 4 and FIG. 5 were expressed as the average value±standard deviation. In FIG. 4 and FIG. 5, “*” indicates the presence of a significant difference compared to Comparative Example 1.

### Transferability According to in vivo Experiments

In the same manner as in the evaluation of the antisense effect, a double-stranded nucleic acid complex was administered into a mouse, the liver of the mouse was taken out, cDNA was synthesized from extracted nucleic acid (DNA/RNA) using an RNA probe which is specific to apolipoprotein B (ApoB)-targeting antisense nucleic acid, and this was used to perform quantitative RT-PCR. Based on the results obtained, the transfer amount (the delivery amount) of the ApoB-targeting antisense nucleic acid was calculated using the amount of sno234 as an internal reference. In addition, the results of each of the groups were compared, and student's T-test was performed after one-way analysis of variance (ANOVA). Multiple comparisons were made with the Bonferroni method. The results are shown in FIG. 5.

### Examples 2 to 6 and Comparative Example 1

A double-stranded nucleic acid complex was produced in the same manner as in Example 1 with the exception that, the first nucleic acid strand having the asymmetric phosphorus atom stereoregulation pattern described in Table 2 was used, and these were used to perform evaluations by in vivo experiments in the same manner as in Example 1. The results are shown in FIG. 4 and FIG. 5.

TABLE 2

| | | 5′-terminal wing region | | Gap region | | 3′-terminal wing region | | | |
|---|---|---|---|---|---|---|---|---|---|
| | | (2 mer) | | (8 mer) | | (3 mer) | | | |
| | | | Absolute | | Absolute | | Absolute | | |
| | | | configurations | | configurations | | configurations | | |
| | | | of asymmetric | | of asymmetric | | of asymmetric | | SEQ |
| | Oligonucleotide | Nucleoside | phosphorus | Nucleoside | phosphorus | Nucleoside | phosphorus | Base | ID |
| | name | type | atoms | type | atoms | type | atoms | length | NO. |
| Example 1 | LNA-ASO 1 | LNA | Rp | Deoxyribose | Rp | LNA | Rp | 13 mer | 7 |
| Example 2 | LNA-ASO 2 | LNA | Rp | Deoxyribose | Sp | LNA | Rp | 13 mer | 7 |
| Example 3 | LNA-ASO 3 | LNA | Rp | Deoxyribose | Mix | LNA | Rp | 13 mer | 7 |
| Example 4 | LNA-ASO 4 | LNA | Sp | Deoxyribose | Mix | LNA | Sp | 13 mer | 7 |
| Example 5 | LNA-ASO 5 | LNA | Mix | Deoxyribose | Rp | LNA | Mix | 13 mer | 7 |
| Example 6 | LNA-ASO 6 | LNA | Mix | Deoxyribose | Sp | LNA | Mix | 13 mer | 7 |
| Comparative | LNA-ASO 7 | LNA | Mix | Deoxyribose | Mix | LNA | Mix | 13 mer | 7 |
| Example 1 | | | | | | | | | |

In Table 2, “Mix” indicates that the absolute configurations of the asymmetric phosphorus atoms are not stereoregulated (non-stereoregulated). That is, LNA-ASO3, for example, in which the asymmetric phosphorus atoms are non-stereoregulated includes ASOs having a total of 128 types of steric structures in which the seven phosphorothioate bonds between the eight nucleotides of the gap region are in the R configuration (Rp) or S configuration (Sp).

The double-stranded nucleic acid complex of Example 1 (Rp-Rp-Rp) and the double-stranded nucleic acid complex of Example 3 (Rp-Mix-Rp), in which a double-stranded nucleic acid complex was prepared by binding Toc-cRNA to a single-stranded LNA/DNA gapmer-type antisense oligonucleotide (ASO) having stereoregulated asymmetric phosphorus atoms, were compared to the double-stranded nucleic acid complex of Comparative Example 1 (Mix-Mix-Mix), and the target gene suppressing effects were respectively confirmed to increase by about 1.6 times and 3.2 times (FIG. 4).

In addition, in the double-stranded nucleic acid complex of Example 1 (Rp-Rp-Rp) and the double-stranded nucleic acid complex of Example 3 (Rp-Mix-Rp), the amount of the complex transferred to the liver was greater than in with the double-stranded nucleic acid complex of Comparative Example 1 (Mix-Mix-Mix) (FIG. 5).

This is because the blood transfer carrier of conventional single-stranded ASO was albumin, and due to the effects of the affinity of the single-stranded ASO with respect to albumin, the amount of the single-stranded ASO (Rp-Rp-Rp) and the amount of the single-stranded (ASO (Rp-Mix-Rp) transferred to the liver were respectively 0.73 and 0.37 in comparison to the amount of the single-stranded ASO (Mix-Mix-Mix) transferred to the liver (not shown in the Figures).

In contrast, in the case of a double-stranded nucleic acid complex in which Toc-cRNA was bonded to a single-stranded ASO, the main transfer carrier in the blood was a high-density lipoprotein (HDL), so the amount transferred improved, which may also contribute to the target gene suppressing effect.

Although Toc-cRNA was bound, the amount of the double-stranded nucleic acid complex of Example 2 (Rp-Sp-Rp) and the double-stranded nucleic acid complex of Example 6 (Mix-Sp-Mix) that was transferred was around ⅓ of that of the double-stranded nucleic acid complex of Comparative Example 1 (Mix-Mix-Mix).

While the S-configuration (Sp) is said to yield better stability than the R-configuration (Rp), the potential for decomposition is considered to be very small.

As described above, it can be seen that the double-stranded nucleic acid complex according to the present disclosure is a double-stranded nucleic acid complex having a designable level of suppression of the expression of a target gene and level of delivery to a target site.

The disclosure of Japanese Patent Application No. 2019-057475, filed Mar. 25, 2019, is incorporated herein by reference in its entirety.

All publications, patent applications, and technical standards mentioned in this specification are herein incorporated by reference to the same extent as if each individual publication, patent application, or technical standard was specifically and individually indicated to be incorporated by reference.

1. A double-stranded nucleic acid complex comprising a first nucleic acid strand and a second nucleic acid strand bonded to each other, the second nucleic acid strand including a complementary region having a base sequence complementary to the first nucleic acid strand,
the first nucleic acid strand including at least one selected from the group consisting of natural nucleosides and non-natural nucleosides, and
at least some of the nucleosides in at least one nucleic acid strand selected from the group consisting of the first nucleic acid strand and the second nucleic acid strand being bonded together by bonds including asymmetric phosphorus atoms, and absolute configurations of the asymmetric phosphorus atoms being regulated.

2. The double-stranded nucleic acid complex according to claim 1, wherein the double-stranded nucleic acid complex comprises a nucleic acid structure that can be recognized by RNase H.

3. The double-stranded nucleic acid complex according to claim 1, wherein the first nucleic acid strand comprises:
two terminal regions each including 2 to 10 consecutive nucleosides extending from a 5′ terminal and a 3′ terminal of the first nucleic acid strand, respectively; and
a middle region that is positioned between the terminal regions and includes at least four nucleosides,
at least some of the nucleosides in at least one region selected from the group consisting of the terminal regions and the middle region being bonded together by bonds including asymmetric phosphorus atoms, and absolute configurations of the asymmetric phosphorus atoms being regulated.

4. The double-stranded nucleic acid complex according to claim 3, wherein at least some of the nucleosides in the terminal regions are bonded together by bonds including asymmetric phosphorus atoms, and an absolute configuration of each asymmetric phosphorus atom is regulated to an S-configuration or an R-configuration.

5. The double-stranded nucleic acid complex according to claim 3, wherein at least some of the nucleosides in the middle region are bonded together by bonds including asymmetric phosphorus atoms, and an absolute configuration of each asymmetric phosphorus atom is regulated to an S-configuration or an R-configuration.

6. The double-stranded nucleic acid complex according to claim 1, wherein the first nucleic acid strand includes at least 4 consecutive deoxyribonucleosides, the second nucleic acid strand includes at least 4 consecutive ribonucleosides, and the double-stranded nucleic acid complex comprises a structure containing at least four consecutive deoxyribonucleoside-ribonucleoside complementary base pairs.

7. The double-stranded nucleic acid complex according to claim 1, wherein the first nucleic acid strand comprises:
a gap region including four or more consecutive natural nucleosides; and
a wing region including consecutive non-natural nucleosides extending from at least one region selected from the group consisting of a 5′-terminal and a 3′-terminal of the gap region.

8. The double-stranded nucleic acid complex according to claim 1, wherein a bond between a non-natural nucleoside in the first nucleic acid strand and another nucleoside adjacent thereto is achieved by a bond including an asymmetric phosphorus atom, and an absolute configuration of the asymmetric phosphorus atom is regulated to an S-configuration or an R-configuration.

9. The double-stranded nucleic acid complex according to claim 1, wherein the non-natural nucleosides in the first nucleic acid strand are sugar-modified nucleosides.

10. The double-stranded nucleic acid complex according to claim 9, wherein the sugar-modified nucleosides include bridged nucleosides.

11. The double-stranded nucleic acid complex according to claim 1, wherein the non-natural nucleosides in the first nucleic acid strand include sugar-modified nucleosides having a 2′-O-methyl group.

12. The double-stranded nucleic acid complex according to claim 1, wherein the bonds including asymmetric phosphorus atoms in at least one nucleic acid strand selected from the group consisting of the first nucleic acid strand and the second nucleic acid strand are phosphorothioate bonds.

13. A double-stranded nucleic acid complex comprising a first nucleic acid strand and a second nucleic acid strand bonded to each other, the second nucleic acid strand including a complementary region having a base sequence complementary to the first nucleic acid strand,
the first nucleic acid strand including:
a gap region including four or more consecutive deoxyribonucleosides, and
wing regions including sugar-modified nucleosides extending from a 5′-terminal and a 3-terminal of the gap region, respectively,
at least some of the nucleosides in the first nucleic acid strand being bonded together by bonds including asymmetric phosphorus atoms, and absolute configurations of the asymmetric phosphorus atoms being regulated, and
the second nucleic acid strand including ribonucleosides.

14. The double-stranded nucleic acid complex according to claim 13, wherein bonds between nucleosides of the wing region are bonds including asymmetric phosphorus atoms in which the absolute configurations of the asymmetric phosphorus atoms are regulated to an R-configuration.

15. The double-stranded nucleic acid complex according to claim 13, wherein bonds between the deoxyribonucleosides are bonds including asymmetric phosphorus atoms in which an absolute configuration of each asymmetric phosphorus atom is regulated to an R-configuration or an S-configuration, or bonds including asymmetric phosphorus atoms in which an absolute configuration of each asymmetric phosphorus atom is not regulated.

16. The double-stranded nucleic acid complex according to claim 13, wherein a base length of the gap region is from 1 to 20 bases, and a base length of the wing region is from 1 to 10 bases.

17. The double-stranded nucleic acid complex according to claim 13, wherein the bonds including asymmetric phosphorus atoms are phosphorothioate bonds.

18. The double-stranded nucleic acid complex according to claim 1, wherein a base length of the first nucleic acid strand is from 8 to 30 bases.

19. The double-stranded nucleic acid complex according to claim 1, wherein the first nucleic acid strand further comprises at least one nucleic acid selected from the group consisting of peptide nucleic acids and morpholino nucleic acids.

20. The double-stranded nucleic acid complex according to claim 1, wherein the second nucleic acid strand further comprises a functional moiety linked to at least one terminal selected from the group consisting of a 3′-terminal and a 5′-terminal of the second nucleic acid strand.

21. The double-stranded nucleic acid complex according to claim 20, wherein the functional moiety has at least one function selected from the group consisting of a labeling function, a purification function, and a targeted delivery function.

22. The double-stranded nucleic acid complex according to claim 20, wherein the functional moiety is linked to the second nucleic acid strand via a cleavable linker moiety.

23. The double-stranded nucleic acid complex according to claim 20, wherein the functional moiety is at least one molecule species selected from the group consisting of a lipid, an antibody, a peptide, and a protein.

24. The double-stranded nucleic acid complex according to claim 23, wherein the lipid is at least one selected from the group consisting of cholesterol, a fatty acid, a lipid-soluble vitamin, a glycolipid, and a glyceride.

25. The double-stranded nucleic acid complex according to claim 23, wherein the lipid is at least one selected from the group consisting of cholesterol, a tocopherol, and a tocotrienol.

26. The double-stranded nucleic acid complex according to claim 1, wherein the second nucleic acid strand further comprises an overhang region positioned at at least one terminal selected from the group consisting of a 5′-terminal and a 3′-terminal of the complementary region.

27. The double-stranded nucleic acid complex according to claim 26, wherein a bond between a nucleoside in the overhang region and another nucleoside adjacent thereto is achieved by a bond including an asymmetric phosphorus atom, and an absolute configuration of the asymmetric phosphorus atom is regulated to an S-configuration or an R-configuration.

28. The double-stranded nucleic acid complex according to claim 26, wherein a base length of the overhang region is at least 1 base.

29. The double-stranded nucleic acid complex according to claim 26, wherein a base length of the second nucleic acid strand in the overhang region is not greater than 30 bases.

30. The double-stranded nucleic acid complex according to claim 26, wherein the overhang region is not an oligonucleotide region for treatment.

31. The double-stranded nucleic acid complex according to claim 26, wherein the complementary region of the second nucleic acid strand does not include at least two consecutive ribonucleosides.

32. The double-stranded nucleic acid complex according to claim 26, wherein the overhang region includes sugar-modified nucleosides and has a base length of from 9 to 12 bases.

33. The double-stranded nucleic acid complex according to claim 26, wherein the overhang region does not include sugar-modified nucleosides, and a base length of the overhang region is from 9 to 17 bases.

34. A pharmaceutical composition comprising the double-stranded nucleic acid complex according to claim 1 and a pharmaceutically acceptable carrier.

35. A method of administering the pharmaceutical composition according to claim 34 to a subject in need thereof, the method comprising administering the pharmaceutical composition to the subject via intravenous route, intraventricular route, intrathecal route, or subcutaneous route.

36. A method of altering a function of a transcription product in a cell, the method comprising administering the pharmaceutical composition according to claim 34 into the cell.

37. A method of changing an expression level of a protein in a cell, the method comprising administering the pharmaceutical composition according to claim 34 into the cell.

38. A method of changing a protein structure in a cell, the method comprising administering the pharmaceutical composition according to claim 34 into the cell.

39-41. (canceled)

42. A method of treating a central nervous system disorder, the method comprising administering the pharmaceutical composition according to claim 34 into a cell.
